(19) United States
(12) Patent Application Publication
(10) Pub. No.: US 2024/0383920 A1
(43) Pub. Date: Nov. 21, 2024

(54) SYNTHESIS OF RAS INHIBITORS

(71) Applicant: Revolution Medicines, Inc., Redwood City, CA (US)

(72) Inventors: Shaoling LI, Sunnyvale, CA (US); Yi Ll, San Jose, CA (US); Paul LOBBEN, Eugene, OR (US); Ross WANG, Fremont, CA (US); Steve BALLMER, Burlinggame, CA (US); Xiaojun HUANG, San Jose, CA (US)

(21) Appl. No.: 18/634,246

(22) Filed: Apr. 12, 2024

Related U.S. Application Data
(60) Provisional application No. 63/459,385, filed on Apr. 14, 2023.

(51) Int. Cl. C07D 498/22; C07D 519/00

(52) U.S. Cl. CPC C07D 498/22; C07D 519/00

(57) ABSTRACT

The present invention relates to Ras inhibitors and to methods for preparing Ras inhibitors.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Mad Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61 K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations, and there remains a need for convenient, scalable synthetic methods thereto.

### SUMMARY OF THF INVENTION

The invention features methods of preparing Compound A, intermediates useful in the synthesis of Compound A, and methods of preparing the intermediates. Compound A, a RAS inhibitor, has the following structure:

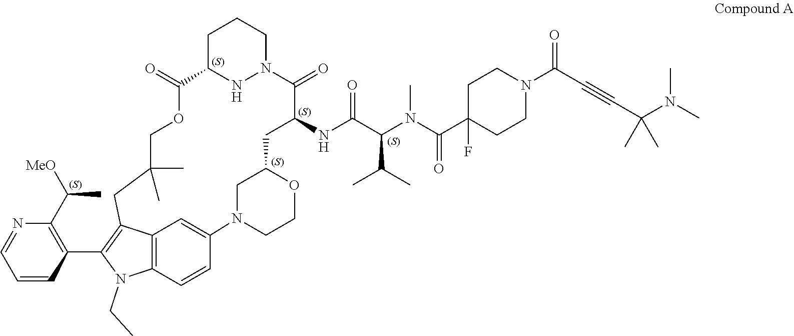

In a first aspect, the disclosure provides a barium salt of Compound 1:

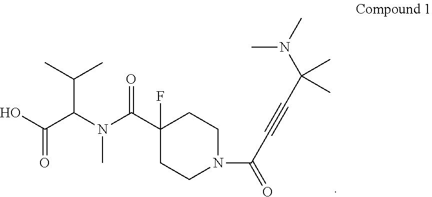 [SMILES: CC(C)C(C(=O)O)N(C)C(=O)C1(F)CCN(C(=O)C#CC(C)(C)N(C)C)CC1]

In some embodiments, the barium salt has a 2:1 ratio of carboxylic acid:barium. In some embodiments, the barium salt has the structure of Compound 2:

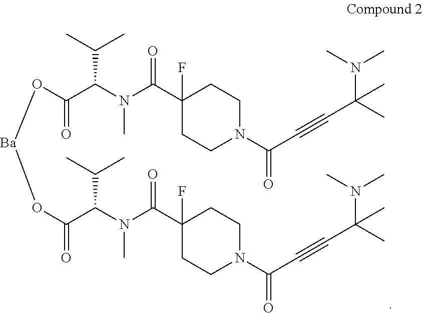

In another aspect, the disclosure provides a compound having the structure of Compound 3:

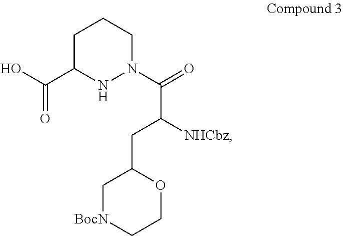 [SMILES: CC(C)(C)OC(=O)N1CCOC(CC(NC(=O)OCc2ccccc2)C(=O)N2CCCC(C(=O)O)N2)C1]

or a salt thereof.

In some embodiments, the compound, or a salt thereof, has the structure of Compound 3a:

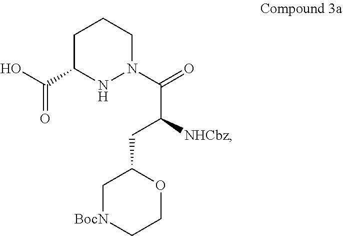 [SMILES: CC(C)(C)OC(=O)N1CCO[C@@H](C[C@H](NC(=O)OCc2ccccc2)C(=O)N2CCC[C@@H](C(=O)O)N2)C1]

In a further aspect, the disclosure provides a method of preparing Compound 4a. The method includes contacting Compound 4 with one or more ketoreductase enzymes:

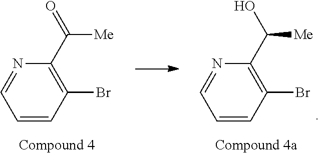 [SMILES: CC(=O)c1ncccc1Br ; C[C@H](O)c1ncccc1Br]

In some embodiments, Compound 4a is formed in at least 85% yield. In some embodiments, the method further includes contacting Compound 4 with glucose dehydrogenase. In some embodiments, the method further includes contacting Compound 4 with glucose. In some embodiments, the method further includes contacting Compound 4 with NADP. In some embodiments of the method of preparing Compound 4a, the contacting is carried out in the presence of a buffer. In some embodiments, the contacting is carried out in the presence of dimethylsulfoxide.

In still another aspect, the disclosure provides a tetramethylethylenediamine (TMEDA) salt of Compound 5:

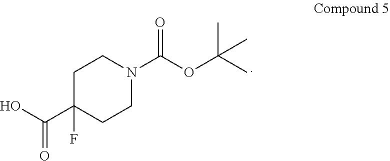 [SMILES: CC(C)(C)OC(=O)N1CCC(F)(C(=O)O)CC1]

In some embodiments, the TMEDA salt has a 2:1 ratio of carboxylic acid:TMEDA.

In a further aspect 1,4-diazabicyclo[2.2.2]octane (DABCO) salt of Compound 5. In some embodiments, the DABCO salt has a 2:1 ratio of carboxylic acid:DABCO.

In another aspect, the disclosure provides a method of preparing Compound 6:

The method includes the steps of:

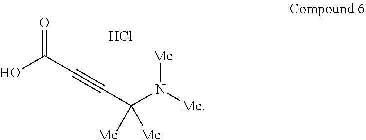 [SMILES: CN(C)C(C)(C)C#CC(=O)O ; Cl]

a) bis-N-methylating Compound 7 to form Compound 7c:

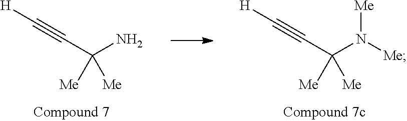 [SMILES: C#CC(C)(C)N ; C#CC(C)(C)N(C)C]

b) carboxylating Compound 7c to form Compound 7a:

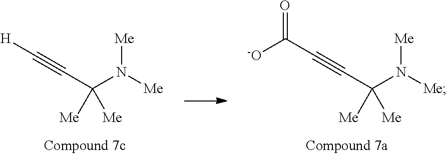

c) protonating Compound 7a to form Compound 7b:

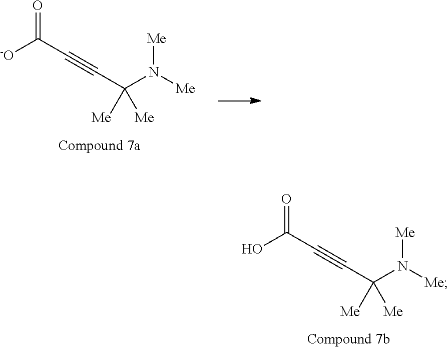

and
d) protonating compound 7b to form Compound 6:

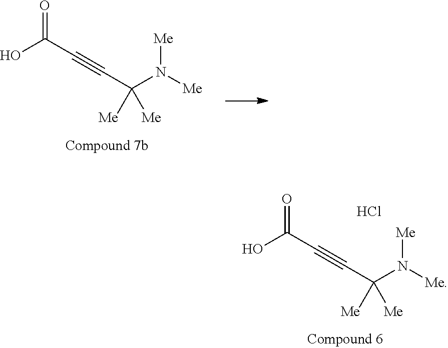 [SMILES: CN(C)C(C)(C)C#CC(=O)O ; CN(C)C(C)(C)C#CC(=O)O ; Cl]

In some embodiments, the bis-N-methylating step (a) includes contacting Compound 7 with an alkylating agent and a reducing agent. In some embodiments, the alkylating agent is formaldehyde, and the reducing agent is sodium triacetoxyborohydride.

In some embodiments, the protonating step (c) includes contacting Compound 7a with acetic acid.

In some embodiments, the protonating step (d) comprises contacting Compound 7b with hydrochloric acid.

In some embodiments, Compound 7a or 7b is used directly in a subsequent chemical step without protonation to form the HCl salt. In some embodiments, Compound 6 is used directly in a subsequent chemical step without isolation or purification from a reaction mixture.

In another aspect, the disclosure provides a method of preparing Compound 8:

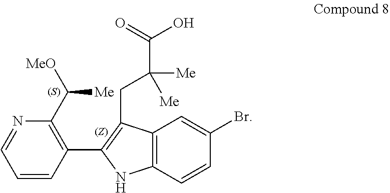

The method includes contacting Compound 9 and Compound 10:

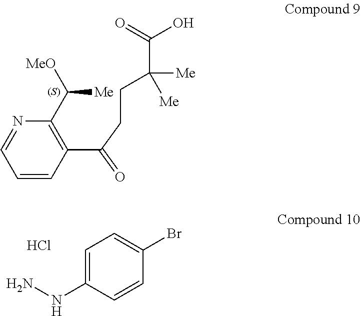

in the presence of acid and water. In some embodiments, the acid is sulfuric acid.

In some embodiments, about 4 equivalents of sulfuric acid are used relative to the amount of Compound 9.

In another aspect, the disclosure provides a method of preparing Compound 8:

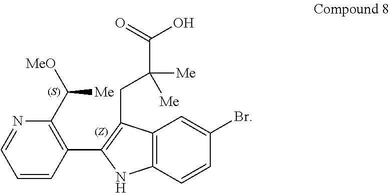

The method includes the steps of: Compound 8
a) coupling Compound 4b and Compound 20 to form Compound 21:

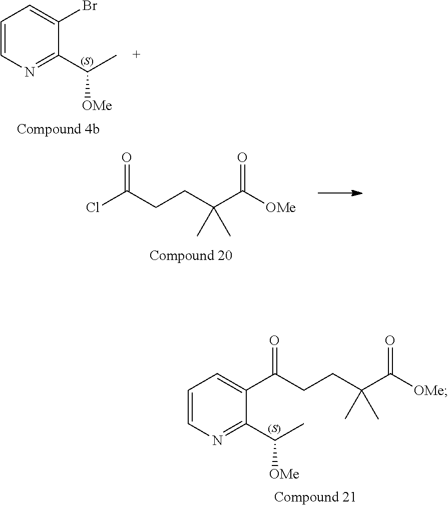

and
b) contacting Compound 21 and Compound 10 to form Compound 8:

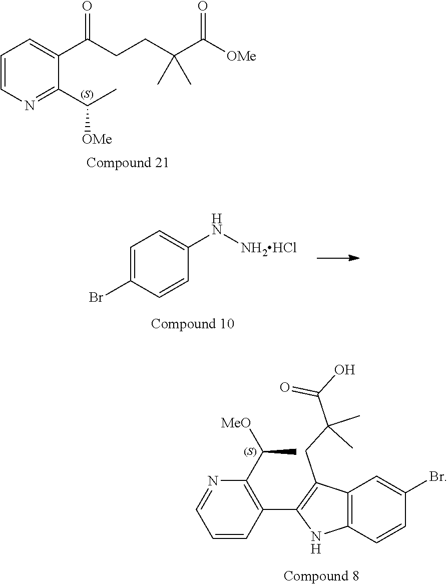

In some embodiments, the coupling step (a) includes contacting Compound 4b with i-PrMgCl·LiCl.

In some embodiments, the contacting step (b) further includes contacting Compound 21 and Compound 10 with sulfuric acid.

In still another aspect, the disclosure provides a hemisulfate (2:1 ratio of Compound 8:sulfate) salt of compound 8:

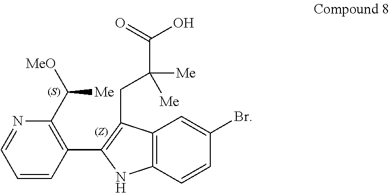

In some embodiments, the disclosure provides a method of preparing the hemisulfate salt of compound 8, the method includes contacting a free base of Compound 8 with sulfuric acid.

In another aspect, the disclosure provides a method of preparing Compound 11a and 11 b,

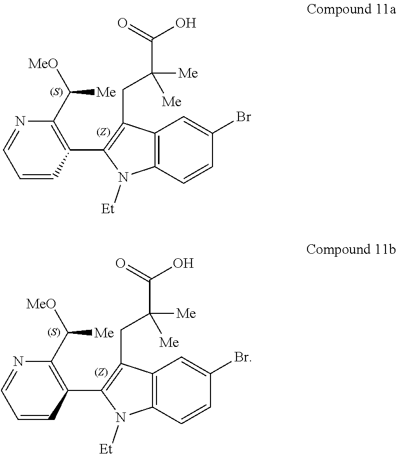

the method including the steps of:
a) reducing Compound 22 to form Compound 23

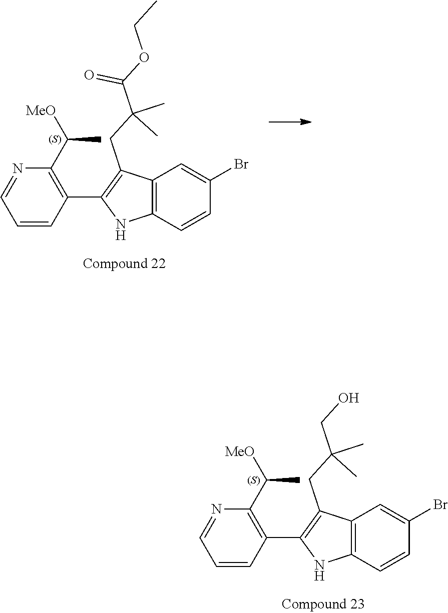

b) alkylating Compound 23 to form a mixture of Compound 11a and 11b

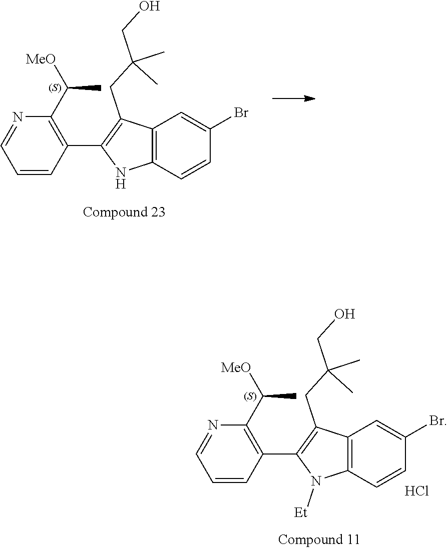

In another aspect, the disclosure provides a method of preparing Compound 11a and 11 b,

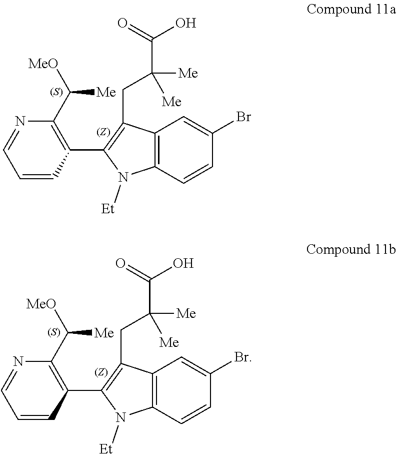

the method including the steps of:
a) alkylating Compound 22 to form Compound 24

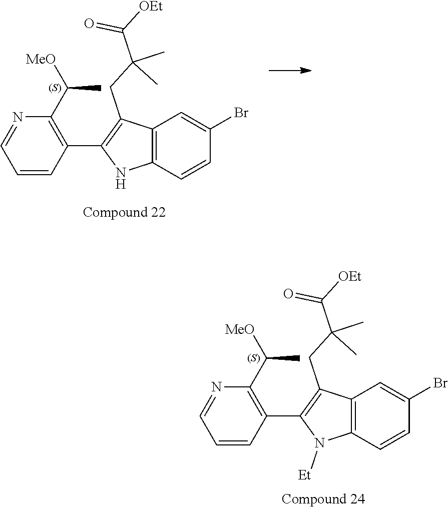

and
b) reducing Compound 24 to form a mixture of Compound 11a and 11b

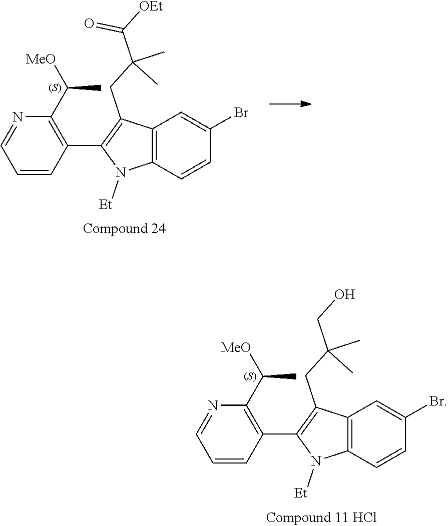

In some embodiments, the alkylating step (a) includes contacting Compound 22 or Compound 23 with diethylsulfate.

In some embodiments, the reducing step (b) includes contacting Compound 22 or Compound 24 with sodium borohydride.

In yet another aspect, the disclosure provides a method of separating Compound 11a and Compound 11b:

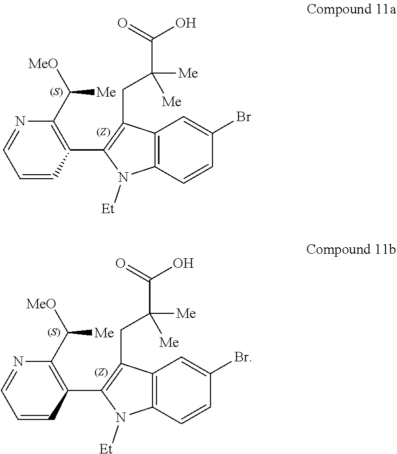

The method includes the steps of:
a) heating a mixture of Compound 11a and Compound 11b in a solvent or mixture of solvents for a period of time;
b) forming a mixture of a salt of Compound 11a and a salt of Compound 11b; and
c) separating the mixture of the salt of Compound 11a and Compound 11b.

In some embodiments, the heating step (a) is carried out in a mixture of xylenes.

In some embodiments, the salt of Compound 11a and Compound 11b is a hydrochloride salt of Compound 11a and Compound 11b.

In some embodiments, the separating is carried out as a flow process.

In a further aspect, the disclosure provides a method of preparing Compound 3a:

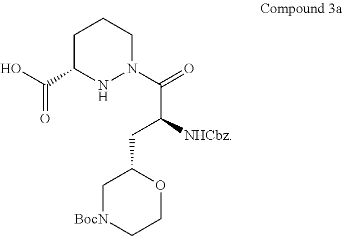 [SMILES: CC(C)(C)OC(=O)N1CCO[C@@H](C[C@H](NC(=O)OCc2ccccc2)C(=O)N2CCC[C@@H](C(=O)O)N2)C1]

The method includes the steps of:
a) deprotonating compound 3b with a chiral base, thereby forming a diastereomeric salt of compound 3b:

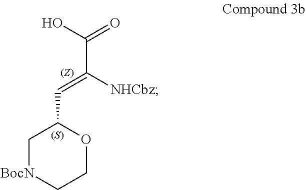

b) reducing the diastereomeric salt of Compound 3b to form Compound 3c:

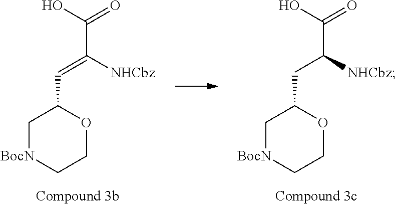 [SMILES: CC(C)(C)OC(=O)N1CCO[C@@H](/C=C(\NC(=O)OCc2ccccc2)C(=O)O)C1 ; CC(C)(C)OC(=O)N1CCO[C@@H](C[C@H](NC(=O)OCc2ccccc2)C(=O)O)C1]

c) coupling Compound 3c with Compound 3d, or a salt thereof, to form Compound 3e;

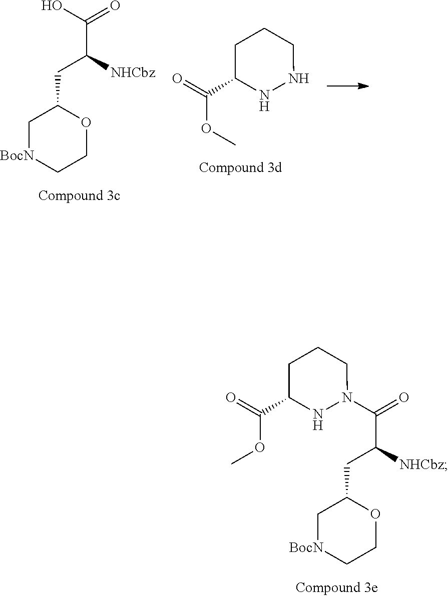

and
d) hydrolyzing Compound 3e to form Compound 3a:

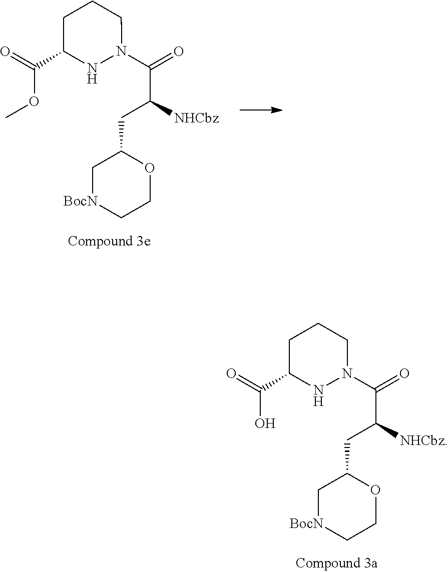 [SMILES: COC(=O)[C@@H]1CCCN(C(=O)[C@H](C[C@H]2CN(C(=O)OC(C)(C)C)CCO2)NC(=O)OCc2ccccc2)N1 ; CC(C)(C)OC(=O)N1CCO[C@@H](C[C@H](NC(=O)OCc2ccccc2)C(=O)N2CCC[C@@H](C(=O)O)N2)C1]

In some embodiments, the chiral base is Compound 3f.

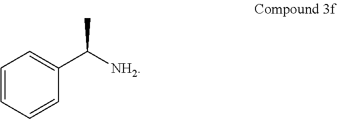

In some embodiments, the method includes protonating Compound 3b prior to the reducing step (b). In some embodiments, the protonating step includes contacting Compound 3b with hydrochloric acid. In some embodiments, hydrochloric acid is in an ethereal solution. In some embodiments, the hydrochloric acid is in a methyl-tert-butyl ether solution.

In some embodiments, the reducing step (b) comprises contacting Compound 3b with hydrogen gas. In some embodiments, the hydrogen gas is at a pressure of about 4 bar. In some embodiments, the reducing step (b) further includes contacting Compound 3b with a rhodium catalyst. In some embodiments, the reducing step (b) further comprises contacting Compound 3b with a chiral ligand. In some embodiments, the rhodium catalyst is Rh(COD)₂OTf. In some embodiments, the chiral ligand is (S,S)-Et-DuPhos. In some embodiments, less than 0.25 mol % of the rhodium catalyst is used relative to the amount of Compound 3b.

In some embodiments, enzymatic chemistry may be utilized to obtain enantiomerically enriched Compound 3a. In some embodiments, Compound 3a is prepared by way of an enzymatic chiral resolution. In some embodiments, Compound 3a is prepared using a phenylammonia vase. In some embodiments, Compound 3a is prepared using a lipase. In some embodiments, Compound 3a is prepared using an amino acid dehydrogenase. The use of enzymatic chemistry may reduce the cost associated with the use of a rhodium catalyst in carrying out an asymmetric hydrogenation. The use of enzymatic chemistry also may lead to increased yield and/or reproducibility compared to an asymmetric hydrogenation.

In an aspect, the disclosure provides method of preparing Compound A:

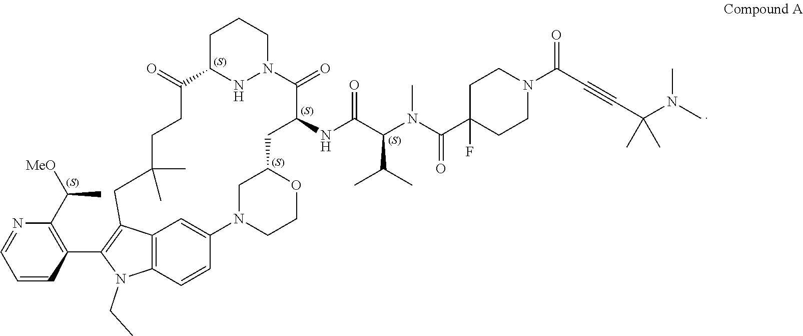

The method includes the steps of:
a) coupling Compound 11a and Compound 3a to form Compound 12:

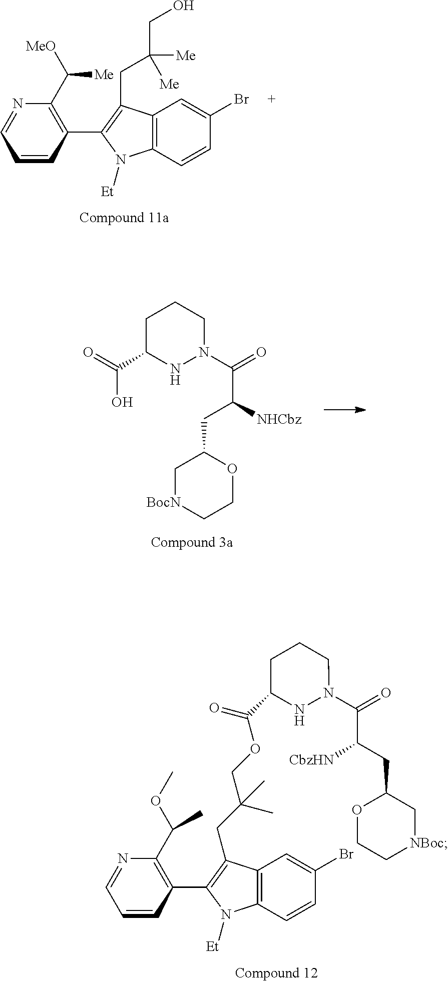

b) deprotecting Compound 12 to form Compound 13:

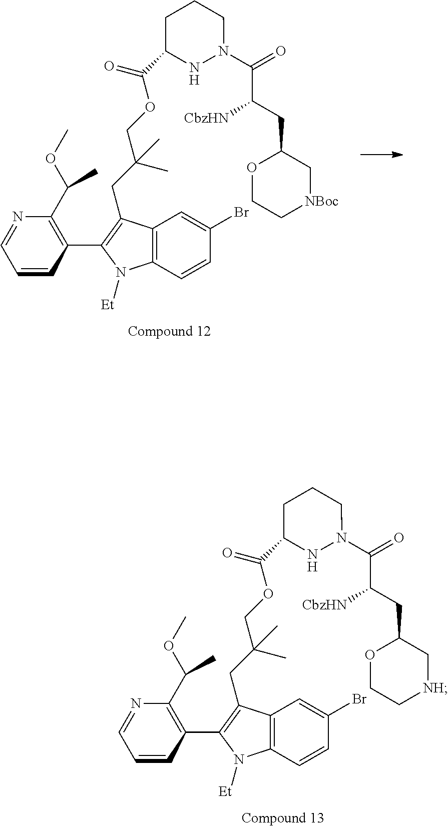

c) coupling Compound 13 to form Compound 14:

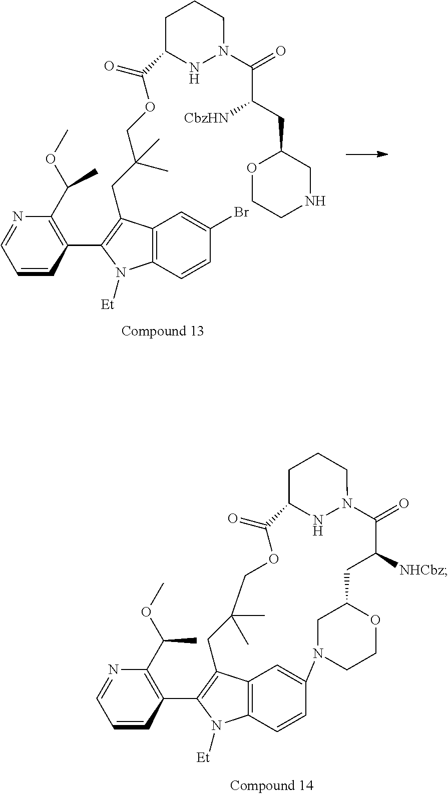

d) deprotecting of Compound 14 to form Compound 15; and

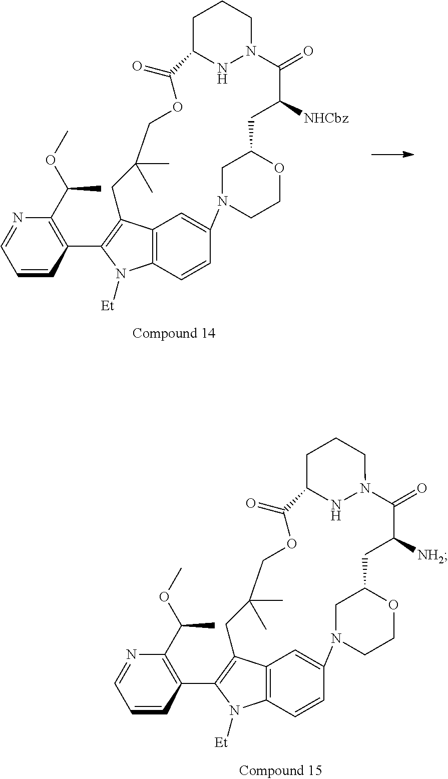

and
e) coupling Compound 15 and Compound 2 to form Compound A:

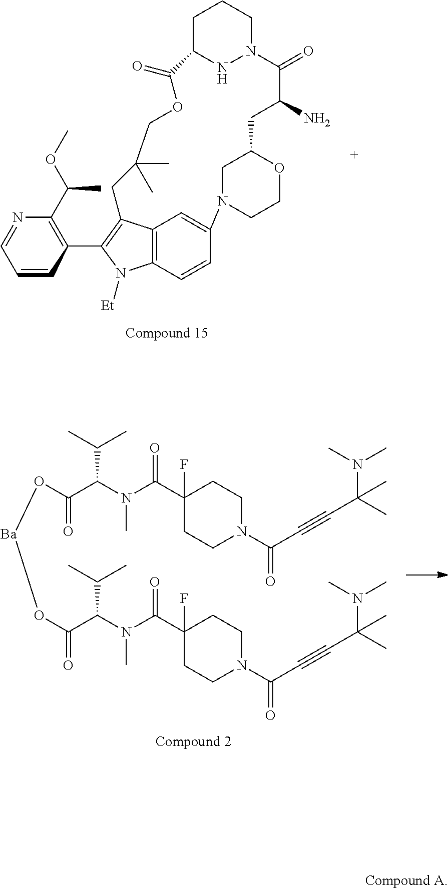

In some embodiments, the method of preparing Compound A further includes the step of purifying Compound A by recrystallizing Compound A. In some embodiments, the recrystallizing includes contacting Compound A with one or more solvents selected from ethyl acetate, water, and diisopropylethylamine, or a mixture thereof. In some embodiments, the recrystallizing is carried out in a mixture of ethyl acetate and n-heptane.

In some embodiments, the coupling step (a) includes contacting Compound 11a and Compound 3a with EDCl and HOBt. In some embodiments, the coupling step (c) is carried out in alumina-treated dioxane. In some embodiments, the coupling step (c) includes contacting Compound 13 with a palladium catalyst. In some embodiments, the method further includes a step of washing a solution of Compound 15 with an aqueous base. In some embodiments, Compound 15 is not isolated or purified prior to the coupling step (e).

In an aspect, the disclosure provides a compound having the structure of Compound 12:

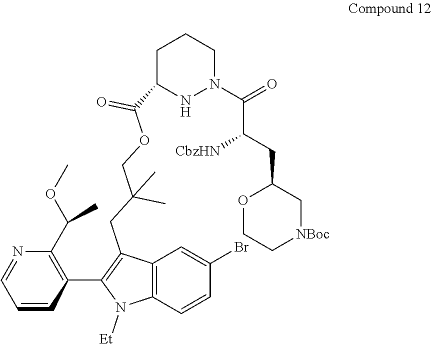

or a salt thereof.

In another aspect, the disclosure provides a compound having the structure of Compound 13:

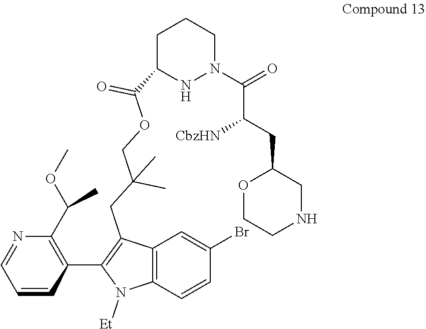

or a salt thereof.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (Iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond. A “compound of the present invention” and similar terms as used herein, whether explicitly noted or not, refers to Ras inhibitors described herein (e.g., Compound A) and intermediates in the synthesis thereto, as well as salts (e.g., pharmaceutically acceptable salts), solvates, hydrates, stereoisomers (including atropisomers), and tautomers thereof.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic add pairs, lactam—lactim pairs, amide—imidic acid pairs, enamine—imine pairs, and annular forms where a proton can occupy two or more positions of a heterocyclic system, such as, 1H— and 3H-imidazole, 1H—, 2H— and 4H-1,2,4-triazole, 1H— and 2H— isoindole, and 1H— and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R°; —(CH₂)₀₋₄OR°; —O(CH₂)₀₋₄R°; —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄—C(O)—N(R°)₂; —(CH₂)₀₋₄—C(O)—N(R°)—S(O)₂—R°; —C(NCN)NR°₂; —(CH₂)₀₋₄C(O)SR°; —(CH₂)₀₋₄C(O)OSiR°₃; —(CH₂)₀₋₄OC(O)R°; —OC(O)(CH₂)₀₋₄SR°; —SC(S)SR°; —(CH₂)₀₋₄SC(O)R°; —(CH₂)₀₋₄C(O)NR°₂; —C(S)NR°₂; —C(S)SR°; —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂₀R°; —(CH₂)₀₋₄₀S(O)₂R°; —S(O)₂NR°₂; —(CH₂)₀₋₄S(O)R°; —N(R°)S(O)₂NR°₂; —N(R°)S(O)₂R°; —N(OR°)R°; —C(NOR°)NR°₂; —C(NH)NR°₂; —P(O)₂R°; —P(O)R°₂; —P(O)(OR°)₂; —OP(O)R°₂; —OP(O)(OR°)₂; —OP(O)(OR°)R°, —SiR°₃; —(C₁₋₄ straight or branched alkylene) O—N(R°)₂; or —(C₁₋₄ straight or branched alkylene)C(O)O—N(R°)₂, wherein each R° may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), may be, independently, halogen, —(CH₂)₀₋₂R^{•}, -(haloR^{•}), —(CH₂)₀₋₂₀H, —(CH₂)₀₋₂OR^{•}, —(CH₂)₀₋₂CH(OR)₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}. —(C₁₋₄ straight or branched alkylene)C(O)OR, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, —NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “Boc” refers to a tert-butyloxycarbonyl or tert-butoxycarbonyl protecting group having the structure

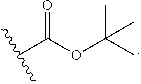 [SMILES: *C(=O)OC(C)(C)C]

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “Cbz” refers to a benzyloxycarbonyl protecting group having the structure of

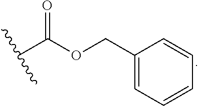 [SMILES: *C(=O)OCc1ccccc1]

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Heteroaryl groups include, but are not limited to, pyridyl, pyrazolyl, benzooxazolyl, benzoimidazoyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂-group.

The term “thiocarbonyl,” as used herein, refers to a —C(S)-group.

The term “ynone,” as used herein, refers to a group comprising the structure

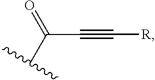

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### BRIEF DESCRIPTION OF THF FIGURE

FIG. 1 is a diagram demonstrating the flow process for separating Compound 11b from the undesired atropisomer Compound 11a.

### DETAILED DESCRIPTION

Provided herein are synthetic methods and intermediates for making the Ras inhibitor Compound A, or a salt thereof. The methods and intermediates can be useful for achieving a higher yield, a higher chemical purity, and/or a higher stereoisomeric purity, and a lower cost for the preparation of Compound A. Further synthetic details are provided in the Examples. The structure of Compound A is shown below.

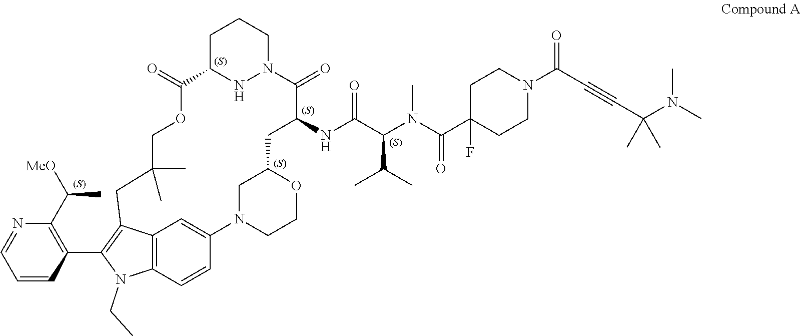

The compounds described herein may be prepared using the methods described herein and/or using known organic, inorganic, or enzymatic processes. The synthetic methods may employ the use of commercially available starting materials or starting materials prepared by processes known to those skilled in the art of organic synthesis. These methods include, but are not limited to, those methods described in the section below and in WO 2021/091982 and WO 2022/235864, the disclosure of each of which is incorporated herein by reference.

### Methods of Synthesis

In a further aspect, the disclosure provides a method of preparing Compound 4a. The method includes contacting Compound 4 with one or more ketoreductase enzymes:

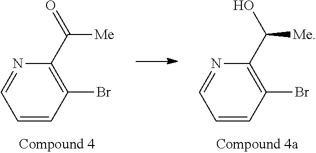 [SMILES: CC(=O)c1ncccc1Br ; C[C@H](O)c1ncccc1Br]

In some embodiments, Compound 4a is formed in at least 85% yield. In some embodiments, the method further includes contacting Compound 4 with glucose dehydrogenase. In some embodiments, the method further includes contacting Compound 4 with glucose. In some embodiments, the method further includes contacting Compound 4 with NADP. In some embodiments of the method of preparing Compound 4a, the contacting is carried out in the presence of a buffer. In some embodiments, the contacting is carried out in the presence of dimethylsulfoxide. In some embodiments, Compound 4a is prepared according to the following scheme:

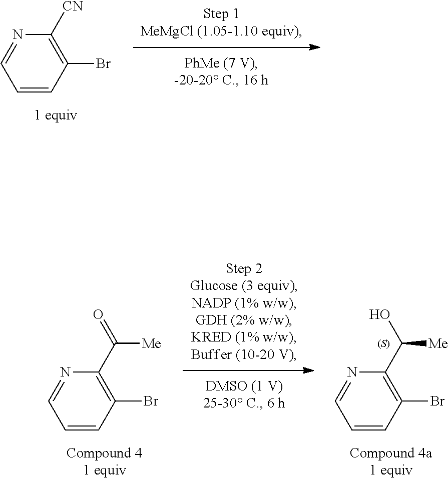

In another aspect, the disclosure provides a method of preparing Compound 6:

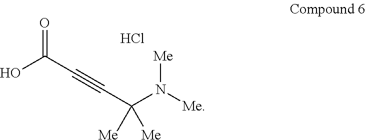 [SMILES: CN(C)C(C)(C)C#CC(=O)O ; Cl]

The method includes the steps of:
a) bis-N-methylating Compound 7 to form Compound 7c:

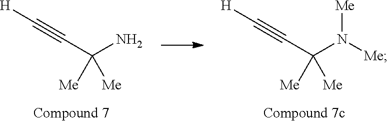 [SMILES: C#CC(C)(C)N ; C#CC(C)(C)N(C)C]

### b) Compound 7 Compound 7c;

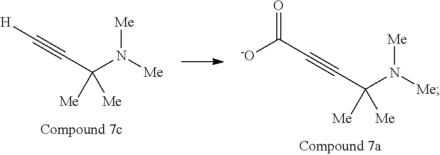 [0123] c) protonating Compound 7a to form Compound 7b:

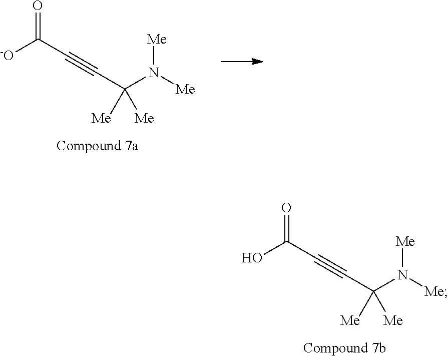

d) protonating Compound 7b to form Compound 6:

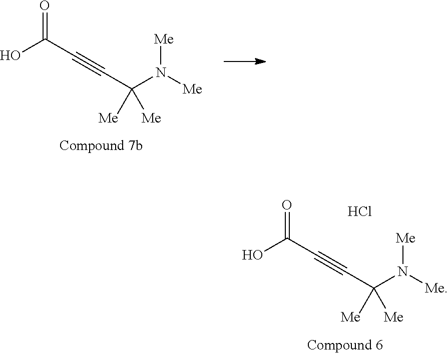 [SMILES: CN(C)C(C)(C)C#CC(=O)O ; CN(C)C(C)(C)C#CC(=O)O ; Cl]

In some embodiments, the bis-N-methylating step (a) includes contacting Compound 7 with an alkylating agent and a reducing agent. In some embodiments, the alkylating agent is formaldehyde, and the reducing agent is sodium triacetoxyborohydride. In some embodiments, the bis-N-methylating step (a) is carried out in a solvent (e.g., an ethereal solvent, e.g., in 2-methyltetrahydrofuran). In some embodiments, the bis-N-methylating step (a) is carried out according to the following scheme:

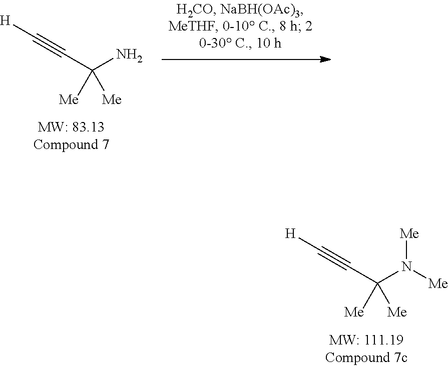 [SMILES: C#CC(C)(C)N ; C#CC(C)(C)N(C)C]

In some embodiments, the carboxylating step (b) includes contacting Compound 7c with a base. In some embodiments, the base is LiHMDS. In some embodiments, the contacting Compound 7c with a base is carried out below room temperature (e.g., below 20° C., below 10° C., below 0° C., below −10° C., below −20° C., below −30° C., below −40° C., below −50° C., or below −00° C.). In some embodiments, the contacting Compound 7c with a base is carried out between −80 and −65° C. In some embodiments, after contacting Compound 7c with a base, Compound 7c is then contacted with carbon dioxide (e.g., carbon dioxide case or dry ice).

In some embodiments, the carboxylating step (b) is carried out according to the following scheme:

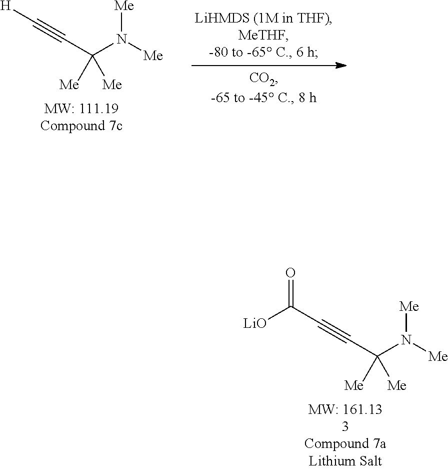

In some embodiments, the protonating step (c) includes contacting Compound 7a with acetic acid. In some embodiments, the protonating step (d) comprises contacting Compound 7b with hydrochloric acid. In some embodiments, Compound 7a or 7b is used directly in a subsequent chemical step without protonation to form the HCl salt. In some embodiments, Compound 6 is used directly in a subsequent chemical step without isolation or purification from a reaction mixture.

In some embodiments, the protonating step (d) and protonating step (d) are carried out according to the following scheme:

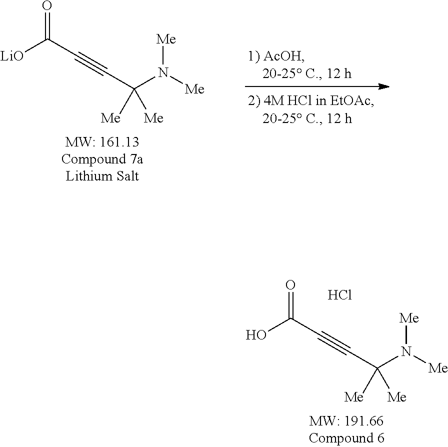

In another aspect, the disclosure provides a method of preparing Compound 8:

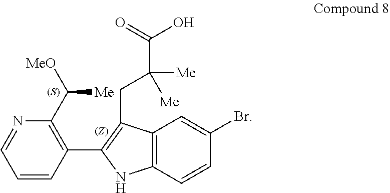

The method includes contacting Compound 9 and Compound 10:

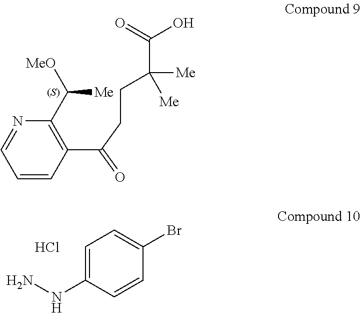

in the presence of acid and water. In some embodiments, the acid is sulfuric acid

In some embodiments, about 4 equivalents of sulfuric acid are used relative to the amount of Compound 9. In some embodiments, the contacting Compound 9 and Compound 10 is carried out above room temperature (e.g., above 20° C., above 30° C., above 40° C., above 50° C., above 60° C., above 70° C., above 80° C., above 90° C., or at 100° C.). In some embodiments, Compound 8 is prepared according to the following scheme:

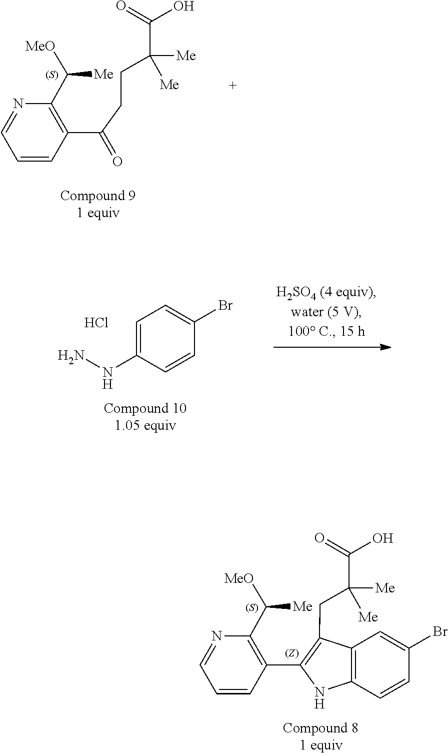

In another aspect, the disclosure provides a method of preparing Compound 8:

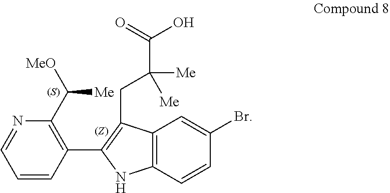

The method includes the steps of:
a) coupling Compound 4b and Compound 20 to form Compound 21:

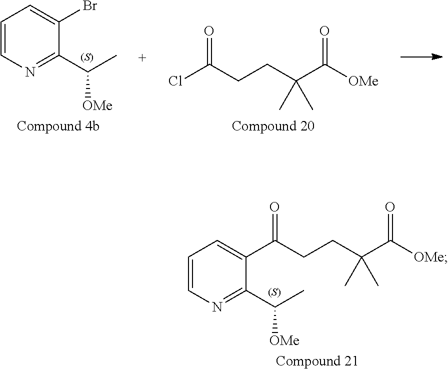

and
b) contacting Compound 21 and Compound 10 to form Compound 8:

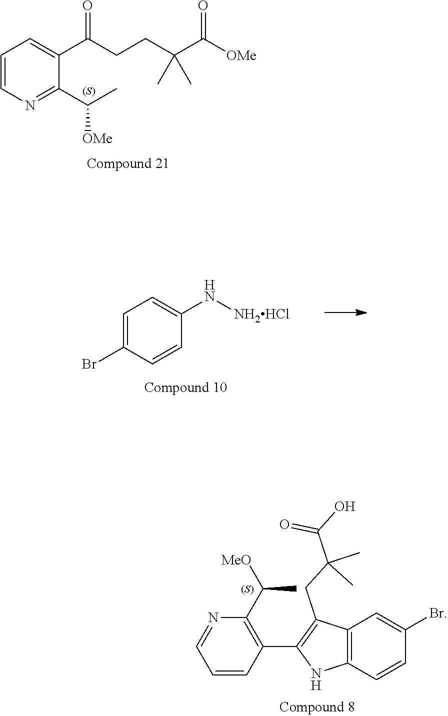

In some embodiments, the coupling step (a) includes contacting Compound 4b with i-PrMgCl·LiCl. In some embodiments, the coupling step (a) includes a zinc source and a copper source. In some embodiments, the zinc source is ZnCl₂. In some embodiments, the copper source is CuCl·2LiCl. In some embodiments, the contacting step (b) further includes contacting Compound 21 and Compound 10 with sulfuric acid. In some embodiments, the contacting step (b) is taken place at a temperature from 100° C.±20° C. In some embodiments, Compound 8 is prepared according to the following scheme:

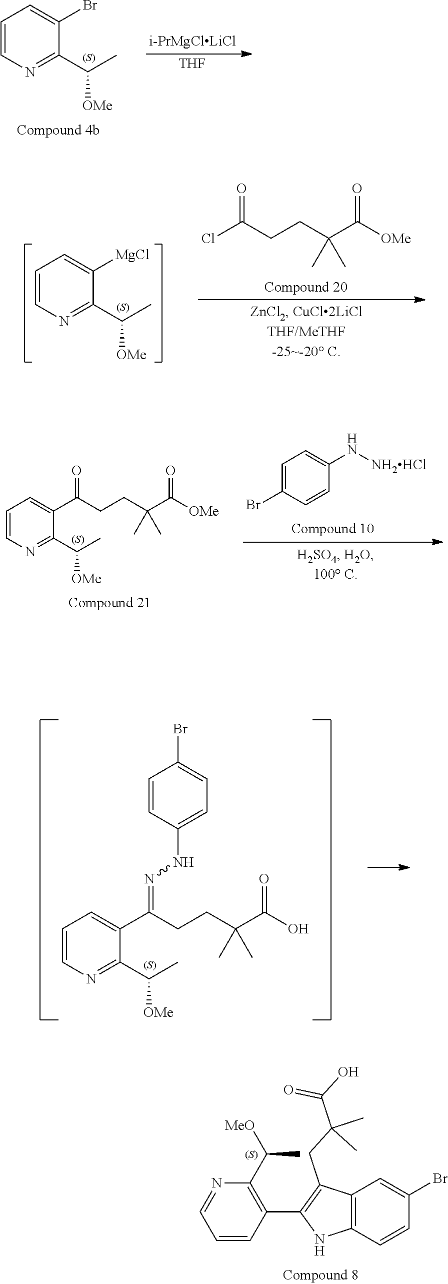

In still another aspect, the disclosure provides a hemisulfate (2:1 ratio of Compound 8:sulfate) salt of compound 8:

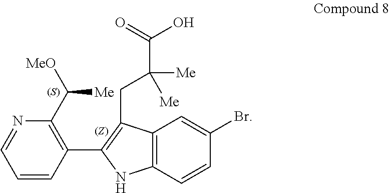

In some embodiments, the disclosure provides a method of preparing the hemisulfate salt of compound 8, the method includes contacting a free base of Compound 8 with sulfuric acid. In some embodiments, the contacting step is heated at 80° C.±15° C. for five hours, then maintained at 30° C.±15° C. for 16 hours.

In another aspect, the disclosure provides a method of preparing Compound 11a and 11 b,

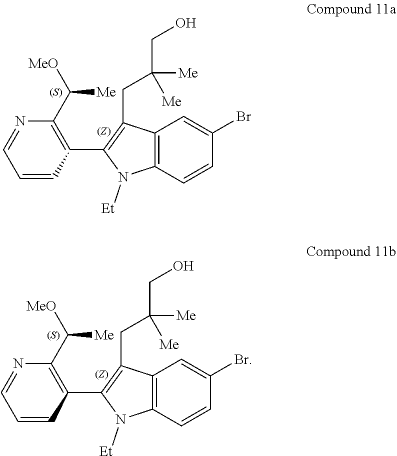

the method including the steps of:
a) reducing Compound 22 to form Compound 23

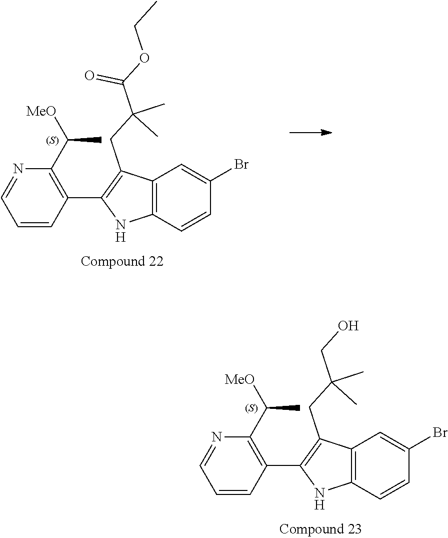

b) alkylating Compound 23 to form a mixture of Compound 11a and 11b

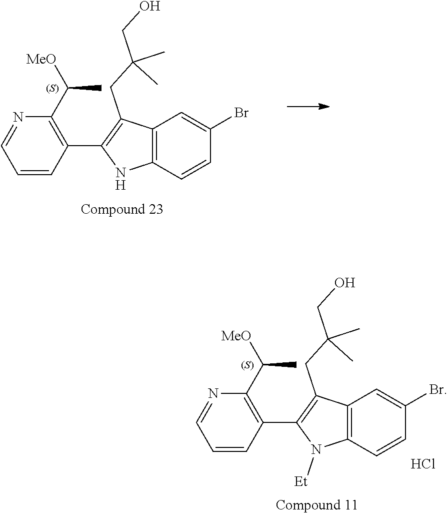

In some embodiments, the reducing step (a) includes a reducing agent. In some embodiments, the reducing agent is sodium borohydride. In some embodiments, the reducing step (a) is maintained at a temperature of 25° C.±10° C. In some embodiments, the reducing step (a) is carried out according to the scheme:

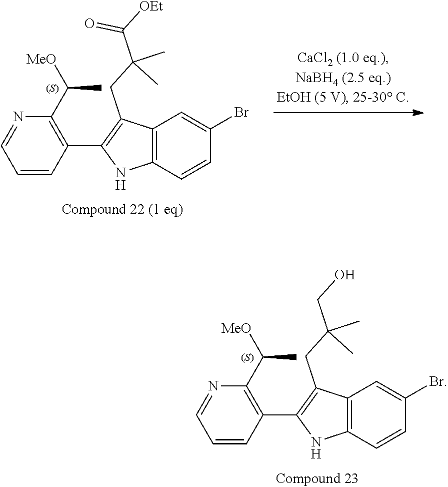

In some embodiments, the alkylating step (b) includes contacting Compound 23 with diethylsulfate (DES). In some embodiments, the alkylating step (b) includes precipitating Compound 11 with an inorganic acid. In some embodiments, the alkylating step (b) is carried out according to the following scheme:

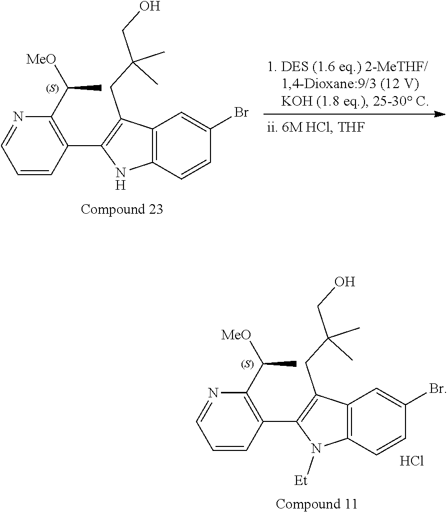

In another aspect, the disclosure provides a method of preparing Compound 11a and 11b,

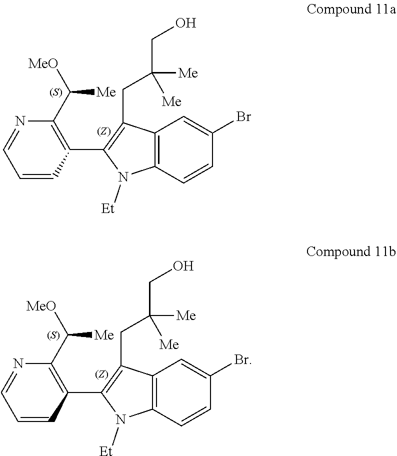

the method including the steps of:
b) alkylating Compound 22 to form Compound 24

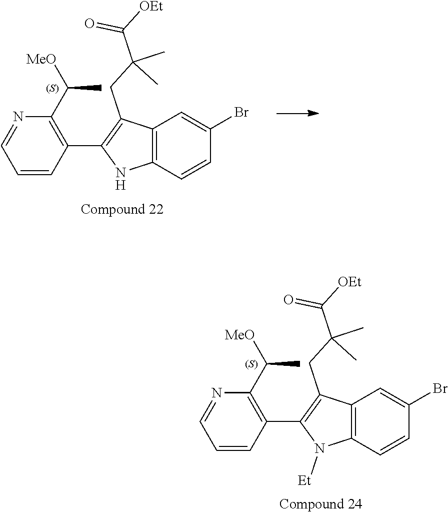

and
b) reducing Compound 24 to form a mixture of Compound 11a and 11b

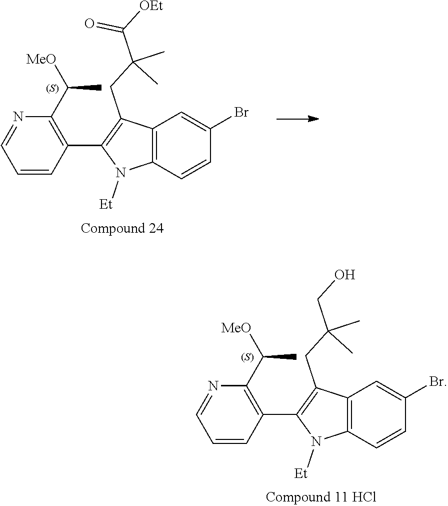

In some embodiments, the alkylating step (a) includes contacting Compound 22 with diethylsulfate. In some embodiments, the alkylating step (a) is carried out according to the following scheme:

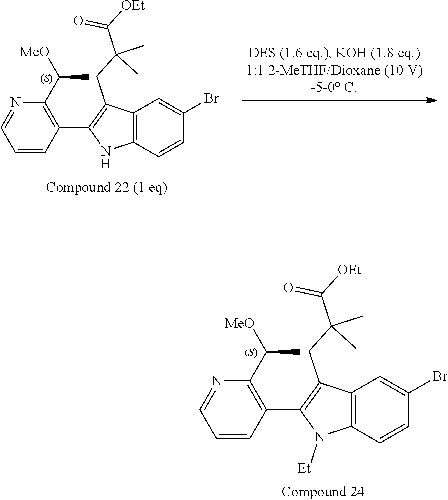

In some embodiments, the reducing step (b) includes contacting Compound 24 with a reducing agent. In some embodiments, the reducing agent is sodium borohydride. In some embodiments, the reducing step (b) includes precipitating Compound 11 with an inorganic acid. In some embodiments, the reducing step (b) is carried out according to the following scheme:

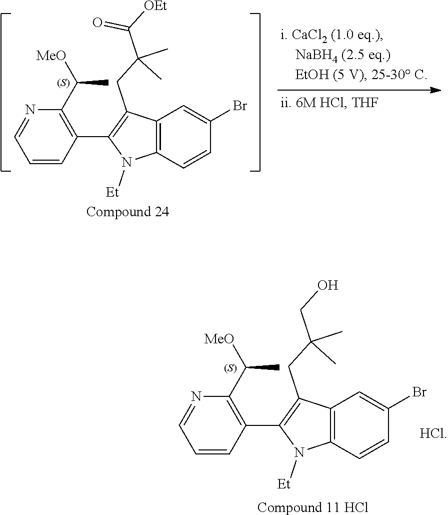

In yet another aspect, the disclosure provides a method of separating Compound 11a and Compound 11b:

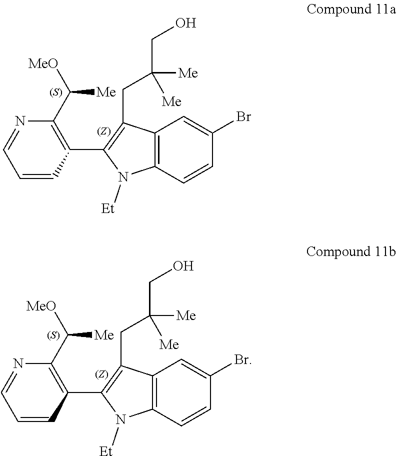

The method includes the steps of:
a) heating a mixture of Compound 11a and Compound 11b in a solvent or mixture of solvents for a period of time;
b) forming a mixture of a salt of Compound 11a and a salt of Compound 11b; and
c) separating the mixture of the salt of Compound 11a and Compound 11b.

In some embodiments, the heating step (a) is carried out in a mixture of xylenes. In some embodiments, the heating step (a) is carried out above 50° C. (e.g., above 75° C., above 100° C., above 125° C., or at 140° C.). In some embodiments, 2 volumes of xylenes are used. In some embodiments, the heating step (a) is carried out for longer than about 1 hour (e.g., for about 1 hour, for about 2 hours, for about 3 hours, or for about 4 hours). In some embodiments, the separating is carried out as a flow process.

In some embodiments, the salt of Compound 11a and Compound 11b is a hydrochloride salt of Compound 11a and Compound 11b. In some embodiments, the hydrochloride salt of Compound 11a and Compound 11b is formed in a solvent. In some embodiments, the solvent is an alcoholic solvent (e.g., isopropanol).

In some embodiments, the method of separating Compound 11a and Compound 11b further includes heating the mixture of the salt of Compound 11a and Compound 11b. In some embodiments, the heating of the mixture of the salts is carried out in a solvent (e.g., an alcoholic solvent such as isopropanol). In some embodiments, the heating of the mixture of the salts is carried out above 50° C. (e.g., above 60° C., above 70° C., or at about 80° C.).

In a further aspect, the disclosure provides a method of preparing Compound 3a:

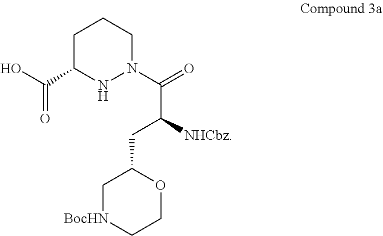

The method includes the steps of:
a) deprotonating compound 3b with a chiral base, thereby forming a diastereomeric salt of compound 3b:

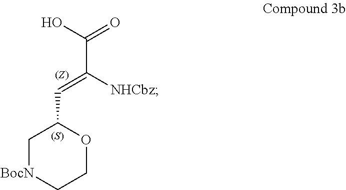

b) reducing the diastereomeric salt of Compound 3b to form Compound 3c:

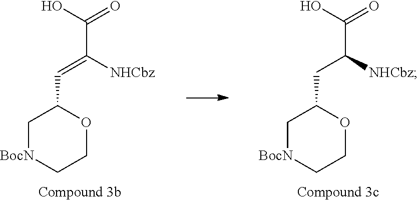 [SMILES: CC(C)(C)OC(=O)N1CCO[C@@H](/C=C(\NC(=O)OCc2ccccc2)C(=O)O)C1 ; CC(C)(C)OC(=O)N1CCO[C@@H](C[C@H](NC(=O)OCc2ccccc2)C(=O)O)C1]

c) coupling Compound 3c with Compound 3d, or a salt thereof, to form Compound 3e;

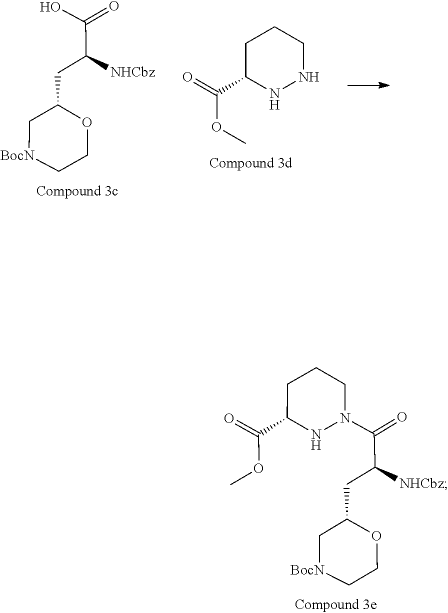

and
d) hydrolyzing Compound 3e to form Compound 3a:

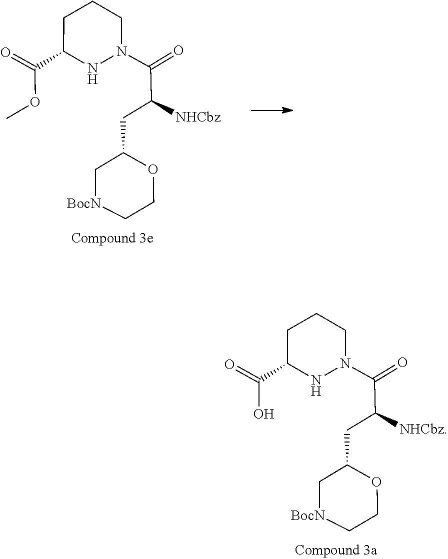 [SMILES: COC(=O)[C@@H]1CCCN(C(=O)[C@H](C[C@H]2CN(C(=O)OC(C)(C)C)CCO2)NC(=O)OCc2ccccc2)N1 ; CC(C)(C)OC(=O)N1CCO[C@@H](C[C@H](NC(=O)OCc2ccccc2)C(=O)N2CCC[C@@H](C(=O)O)N2)C1]

In some embodiments, the chiral base is Compound 3f.

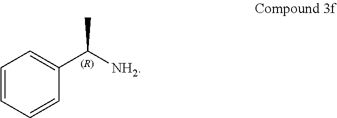

In some embodiments, the 3b prior to the reducing step (b). In some embodiments, the protonating step includes contacting Compound 3b with hydrochloric acid. In some embodiments, hydrochloric acid is in an ethereal solution. In some embodiments, the hydrochloric acid is in a methyl-tert-butyl ether solution.

In some embodiments, the reducing step (b) comprises contacting Compound 3b with hydrogen gas. In some embodiments, the hydrogen gas is at a pressure above 1 bar (e.g., above 2 bar, above 3 bar, about 2 bar, about 3 bar, or about 4 bar). In some embodiments, the hydrogen gas is at a pressure of about 4 bar. In some embodiments, the reducing step (b) further includes contacting Compound 3b with a rhodium catalyst. In some embodiments, the reducing step (b) further comprises contacting Compound 3b with a chiral ligand. In some embodiments, the rhodium catalyst is Rh(COD)₂OTf. In some embodiments, the chiral ligand is (S,S)-Et-DuPhos. In some embodiments, less than 10 mol % (e.g., less than 5 mol %, less than 2.5 mol %, less than 1 mol %, less than 0.75 mol %, less than 0.5 mol %, or less than 0.25 mol %) of the rhodium catalyst is used relative to the amount of Compound 3b. In some embodiments, less than 0.25 mol % (e.g., about 0.20 mol %, about 0.15 mol %, or about 0.10 mol %) of the rhodium catalyst is used relative to the amount of Compound 3b. In some embodiments, the reducing step (c) is carried out in a solvent (e.g., an alcoholic solvent such as methanol). In some embodiments, the reducing step (c) is carried out to the following scheme:

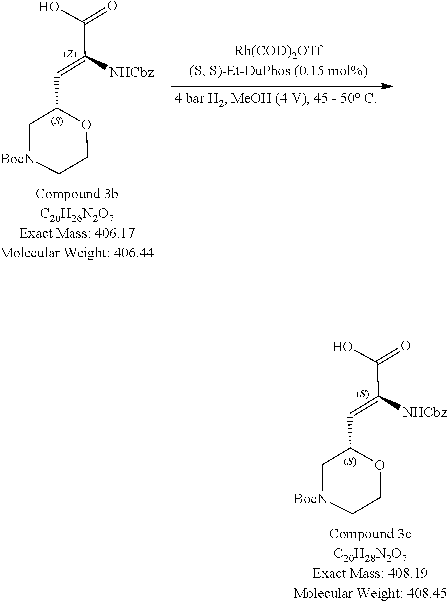

In some embodiments, the coupling step (d) includes contacting Compound 3c and Compound 3d with a coupling reagent (e.g., EDCl). In some embodiments, the coupling step (d) further includes contacting Compound 3c and Compound 3d with HOBt.

In some embodiments, the coupling step (d) is carried out according to the following scheme:

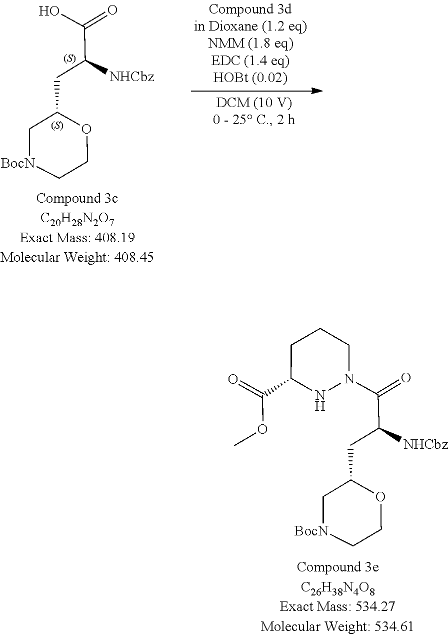

In some embodiments, the hydrolyzing step (e) includes contacting Compound 3e with a hydroxide salt. In some embodiments, the hydroxide salt is lithium hydroxide. In some embodiments, the hydrolyzing step (e) is carried out according to the following scheme:

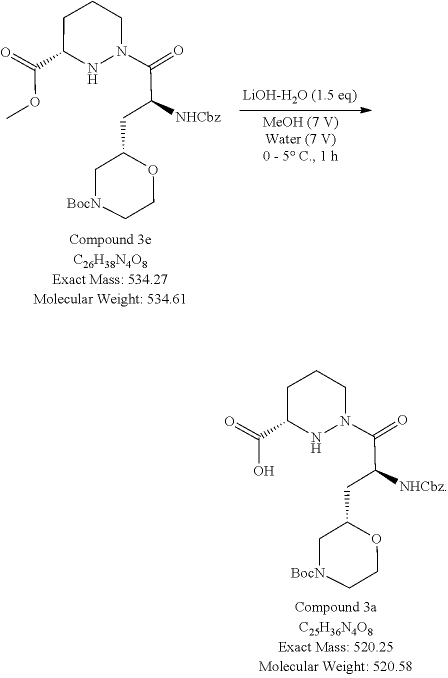 [SMILES: COC(=O)[C@@H]1CCCN(C(=O)[C@H](C[C@H]2CN(C(=O)OC(C)(C)C)CCO2)NC(=O)OCc2ccccc2)N1 ; CC(C)(C)OC(=O)N1CCO[C@@H](C[C@H](NC(=O)OCc2ccccc2)C(=O)N2CCC[C@@H](C(=O)O)N2)C1]

In some embodiments, enzymatic chemistry may be utilized to obtain enantiomerically enriched Compound 3a. In some embodiments, Compound 3a is prepared by way of an enzymatic chiral resolution. In some embodiments, Compound 3a is prepared using a phenylammonia vase. In some embodiments, Compound 3a is prepared using a lipase. In some embodiments, Compound 3a is prepared using an amino acid dehydrogenase. The use of enzymatic chemistry may reduce the cost associated with the use of a rhodium catalyst in carrying out an asymmetric hydrogenation. The use of enzymatic chemistry also may lead to increased yield and/or reproducibility compared to an asymmetric hydrogenation.

In an aspect, the disclosure provides method of preparing Compound A:

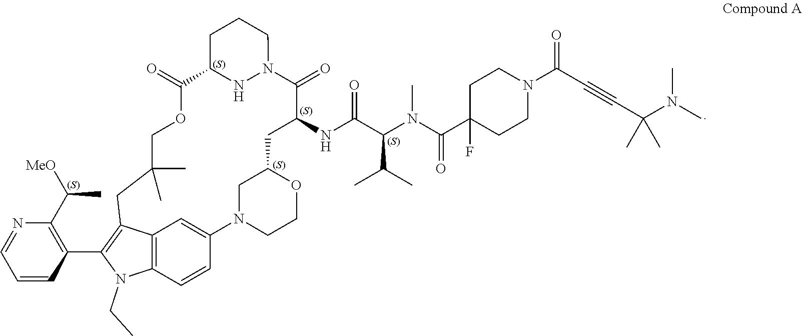

The method includes the steps of:
a) coupling Compound 11a and Compound 3a to form Compound 12:

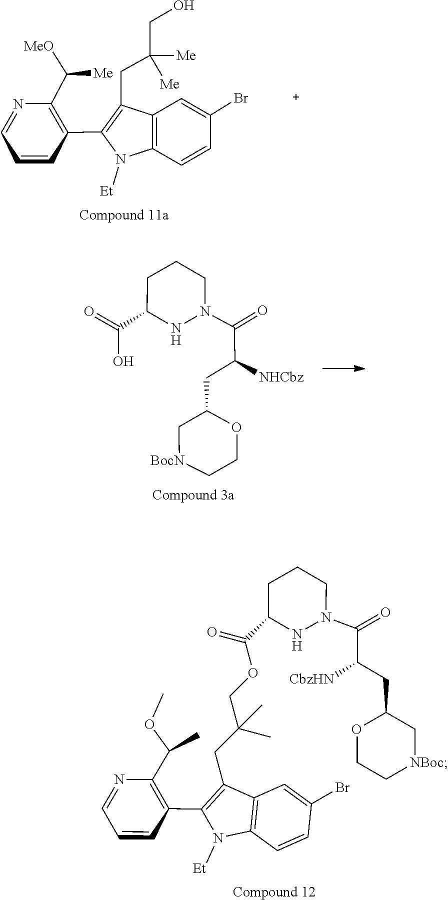

b) deprotecting Compound 12 to form Compound 13:

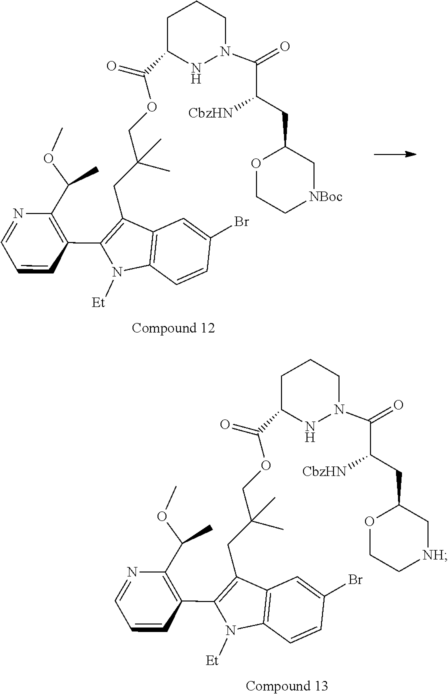

c) coupling Compound 13 to form Compound 14:

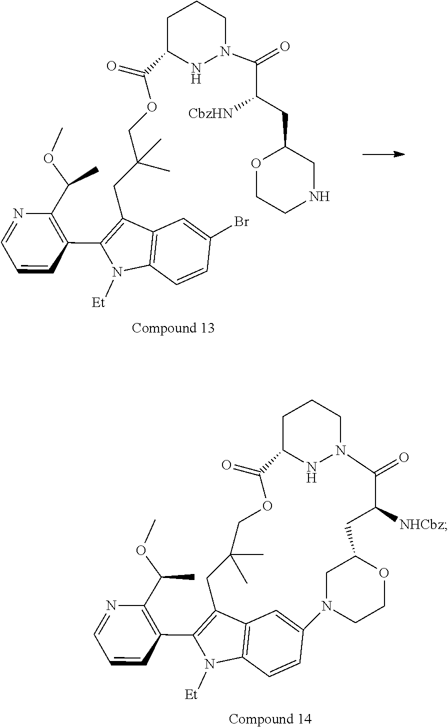

e) deprotecting of Compound 14 to form Compound 15:

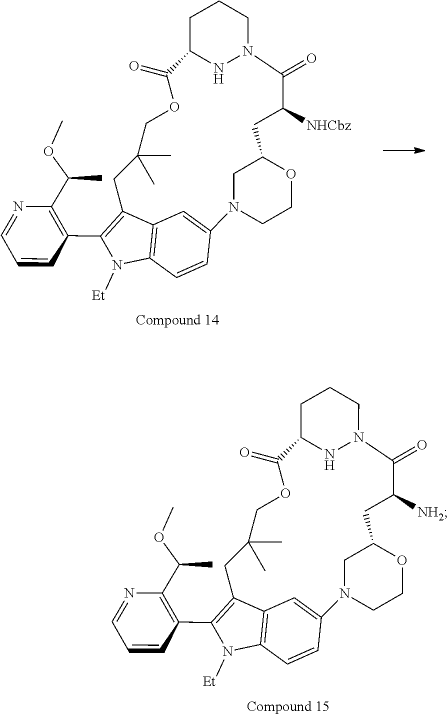

and
e) coupling Compound 15 and Compound 2 to form Compound A:

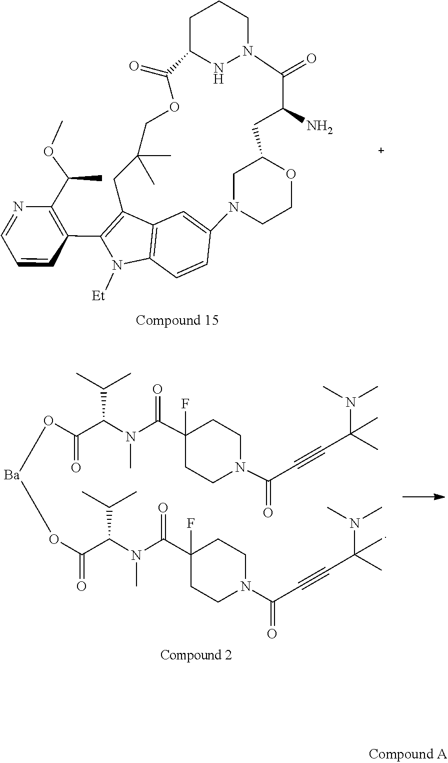

In some embodiments, the coupling step (a) includes contacting Compound 11a and Compound 3a with EDCl and HOBt. In some embodiments, the coupling step (a) further includes contacting Compound 11a and Compound 3a with one or more bases. In some embodiments, the coupling step (a) includes contacting Compound 11a and Compound 3a with DIPEA. In some embodiments, the coupling step (a) includes contacting Compound 11a and Compound 3a with DMAP. In some embodiments, the coupling step (a) is carried out according to the following scheme:

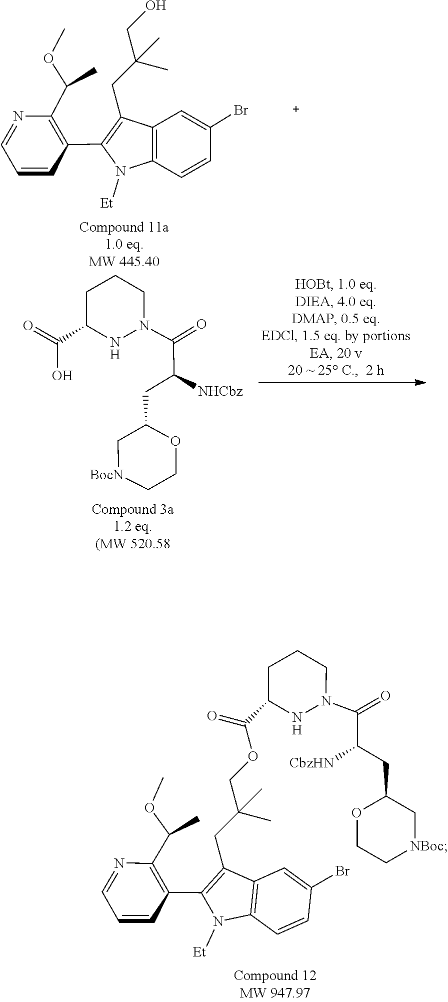

In some embodiments, the deprotecting step (b) includes contacting Compound 12 with an acid. In some embodiments, the acid is hydrochloric acid.

In some embodiments, the deprotecting step (b) is carried out according to the following scheme:

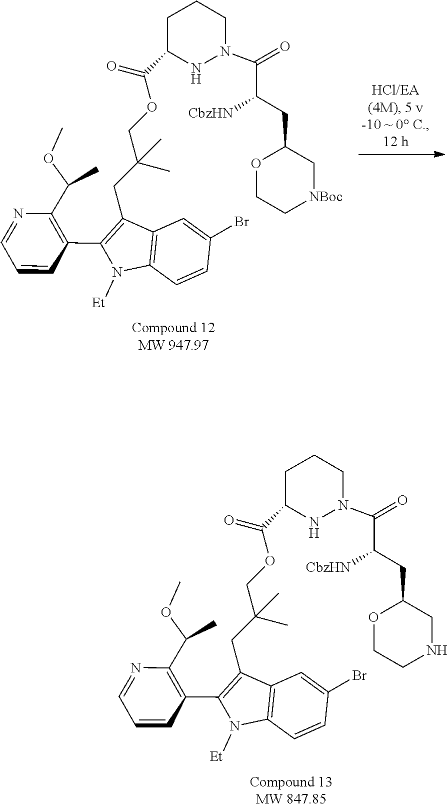

In some embodiments, the coupling step (c) is carried out in alumina-treated dioxane. In some embodiments, the coupling step (c) includes contacting Compound 13 with a palladium catalyst. In some embodiments, the coupling step (c) includes contacting Compound 13 with a base (e.g., a carbonate base such as cesium carbonate). In some embodiments, the coupling step (c) is carried out in a solvent (e.g., an ethereal solvent such as 1,4-dioxane). In some embodiments, the coupling step (c) is carried out above room temperature (e.g., above 50° C., above 60° C., above 70° C., or above 80° C.). In some embodiments, the coupling step (c) is carried out between 8° and 90° C. In some embodiments, the coupling step (c) is carried out according to the following scheme:

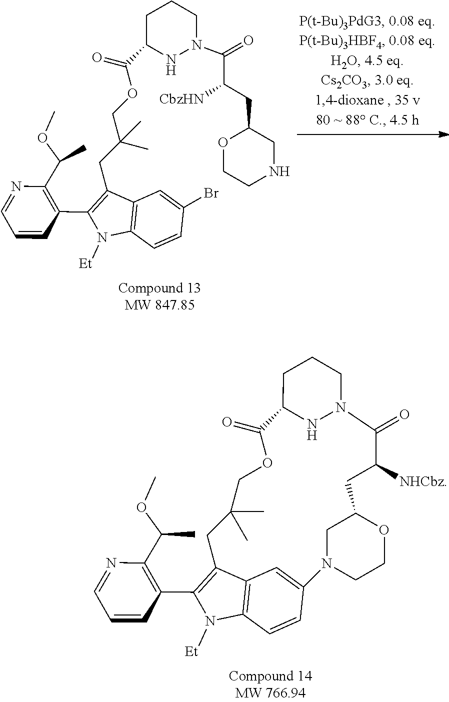

In some embodiments, the deprotecting step (d) includes contacting Compound 14 with hydrogen gas. In some embodiments, the hydrogen gas is at a pressure above 1 atm (e.g., above 2 atm, above 3 atm, above 4 atm). In some embodiments, the hydrogen gas is at a pressure of about 5 atm. In some embodiments, the deprotecting step (d) further includes contacting Compound 14 with a palladium catalyst. In some embodiments, the palladium catalyst is palladium on carbon. In some embodiments, the method further includes a step of washing a solution of Compound 15 with an aqueous base (e.g., an aqueous carbonate base such as sodium carbonate). In some embodiments, Compound 15 is not isolated or purified prior to the coupling step (e).

In some embodiments, the deprotecting step (d) is carried out according to the following scheme:

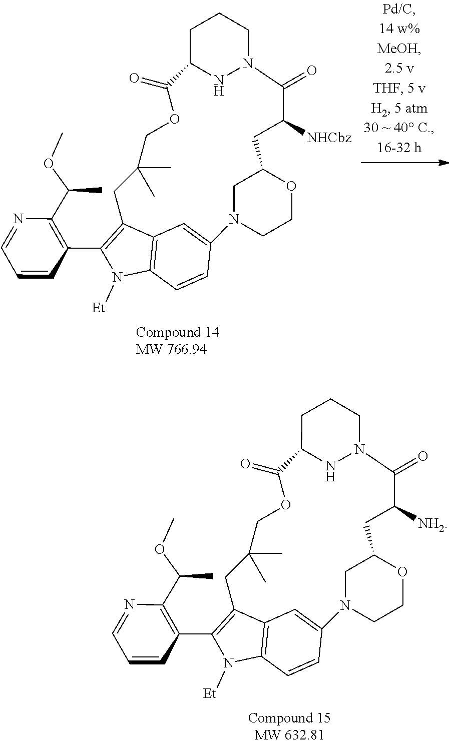

In some embodiments, the coupling step (e) includes contacting Compound 15 and Compound 2 with a coupling reagent (e.g., BOP or PyBOP). In some embodiments, the coupling step (e) includes contacting Compound 15 and Compound 2 with a base (e.g., DIPEA). In some embodiments, the coupling step (e) is carried out according to the following scheme:

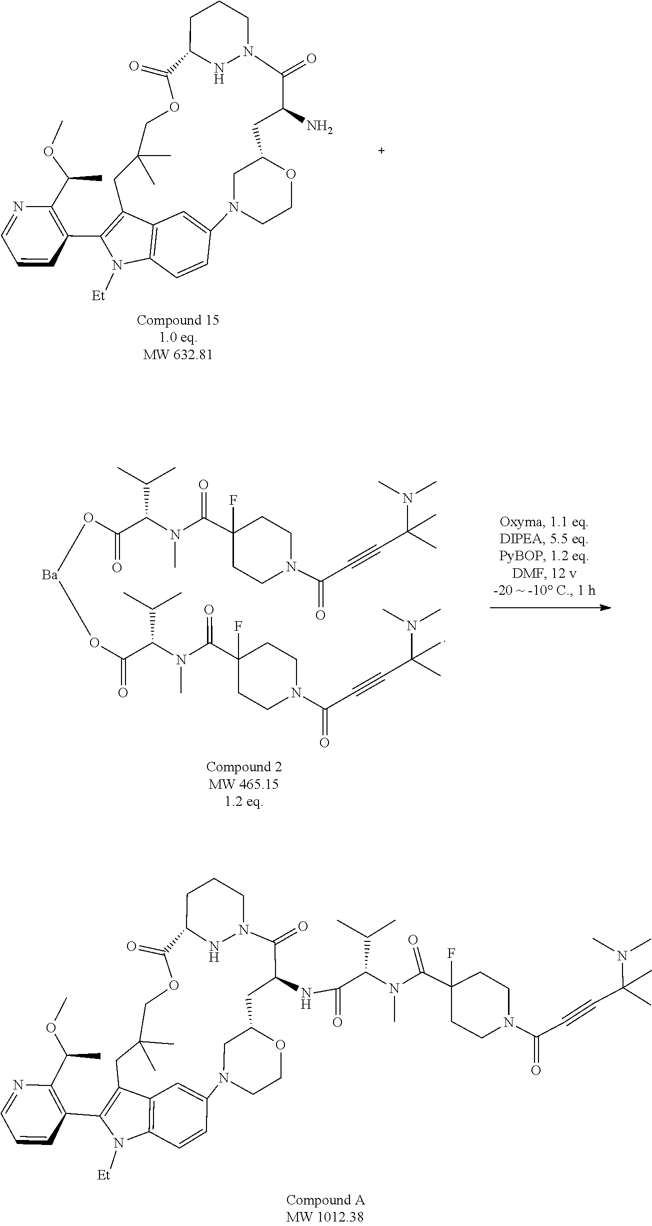

In some embodiments, the method of preparing Compound A further includes the step of purifying Compound A by recrystallizing Compound A. In some embodiments, the recrystallizing includes contacting Compound A with one or more solvents selected from ethyl acetate, water, and diisopropylethylamine, or a mixture thereof. In some embodiments, the recrystallizing is carried out in a mixture of ethyl acetate and n-heptane.

### Compounds and Intermediates

In an aspect, the disclosure provides a barium salt of Compound 1:

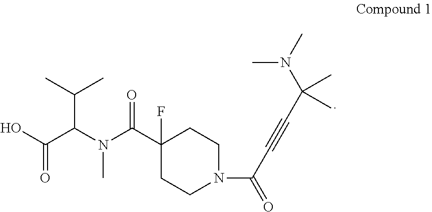 [SMILES: CC(C)C(C(=O)O)N(C)C(=O)C1(F)CCN(C(=O)C#CC(C)(C)N(C)C)CC1]

In some embodiments, the barium salt has a 2:1 ratio of carboxylic acid:barium. In some embodiments, the barium salt has the structure of Compound 2:

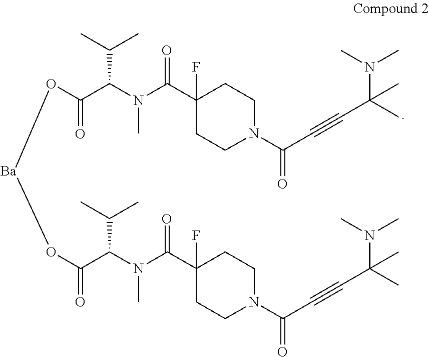

In another aspect, the disclosure provides a compound having the structure of Compound 3:

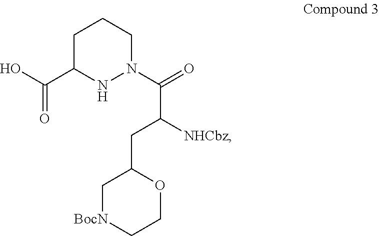 [SMILES: CC(C)(C)OC(=O)N1CCOC(CC(NC(=O)OCc2ccccc2)C(=O)N2CCCC(C(=O)O)N2)C1]

or a salt thereof. In some embodiments, the compound, or a salt thereof, has the structure of Compound 3a:

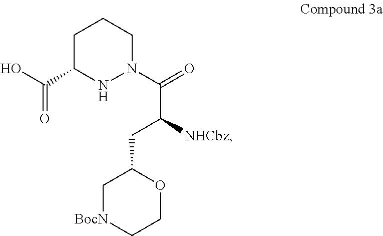 [SMILES: CC(C)(C)OC(=O)N1CCO[C@@H](C[C@H](NC(=O)OCc2ccccc2)C(=O)N2CCC[C@@H](C(=O)O)N2)C1]

In still another aspect, the disclosure provides a tetramethylethylenediamine (TMEDA) salt of Compound 5:

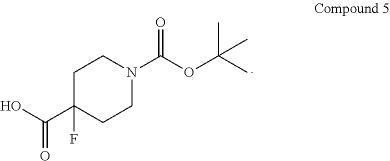 [SMILES: CC(C)(C)OC(=O)N1CCC(F)(C(=O)O)CC1]

In some embodiments, the TMEDA salt has a 2:1 ratio of carboxylic acid:TMEDA.

In a further aspect 1,4-diazabicyclo[2.2.2]octane (DABCO) salt of Compound 5. In some embodiments, the DABCO salt has a 2:1 ratio of carboxylic acid:DABCO.

In still another aspect, the disclosure provides a hemisulfate (2:1 ratio of Compound 8:sulfate) salt of compound 8:

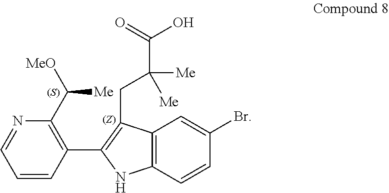

In an aspect, the disclosure provides a compound having the structure of Compound 12:

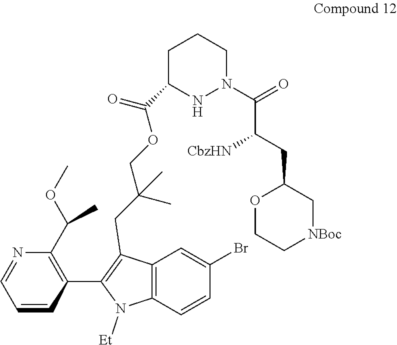

or a salt thereof.

In another aspect, the disclosure provides a compound having the structure of Compound 13:

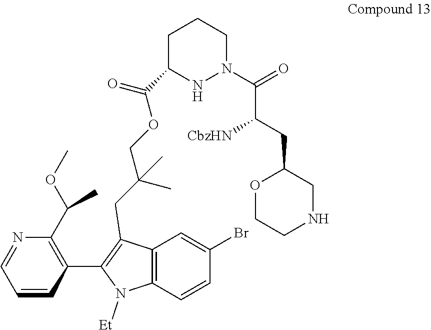

or a salt thereof.

### Examples

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Synthetic Procedure for Compound 4b—(S)-3-bromo-2-(1-methoxyethyl)pyridine

Detailed below is a general synthetic procedure for Compound 4a—(S)-3-bromo-2-(1-methoxyethyl)pyridine.

### Synthesis of Compound 4b—(S)-3-bromo-2-(1-methoxyethyl)pyridine

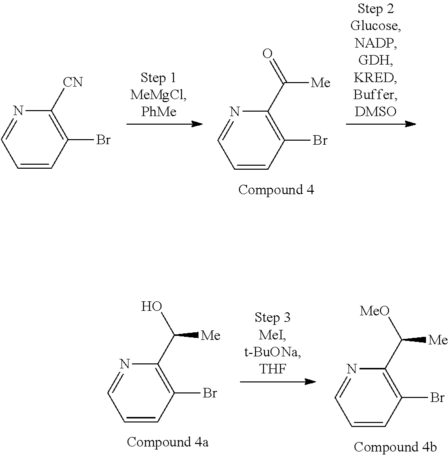 [SMILES: N#Cc1ncccc1Br ; CC(=O)c1ncccc1Br ; C[C@H](O)c1ncccc1Br ; CO[C@@H](C)c1ncccc1Br]

### Part 1—Synthesis of Compound 4—1-(3-bromopyridin-2 yl)ethan-1-one

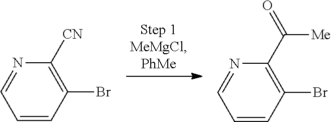 [SMILES: N#Cc1ncccc1Br ; CC(=O)c1ncccc1Br]

To a reactor was charged toluene (2,100 L, 7 V) and 3-bromopicolinonitrile (300 kg, 1,639 mol, 1 equiv). The resulting mixture was cooled to, and maintained at, −20° C. To this was charged MeMgCl (3 M in THF, 601 L, 1,803 mol, 1.1 equiv). The resulting mixture was heated to, and maintained at, 10-20° C. for 16 hours at which point HPLC analysis showed reaction completion.

The reaction mixture was charged into pre-cooled (−10 to 0° C.) 4 M aqueous HCl (1,070 L, 2.6 equiv) at −10 to 10° C. and resulting mixture was maintained at 15-25° C. for 30 minutes. The phases were separated, and the aqueous phase extracted with toluene (600 L×5, 2 V×5). The combined organic layers were washed with saturated aqueous NaHCO₃ (100 L, 0.3 V) and then concentrated (50-60° C., −0.08 MPa) to ˜200 L (0.7 V) to afford crude 1-(3-bromopyridin-2-yl)ethan-1-one (Compound 4a) (346 kg, 93.7% a/a purity, 83.4% w/w assay, 88% yield) as a brown oil which was used directly in the next step.

TABLE 1
HPLC Method for Part 1 of Example 1
HPLC method:

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| | Time (min) | A % | B % |
| Gradient: | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector | 210 nm |
| Wavelength: | |
| Column Temperature: | 15° C. |
| Retention Times: | 3-bromopicolinonitrile: 8.5 min |
| | 1-(3-bromopyridin-2-yl)ethan-1-one: 8.1 min |

LRMS (ESI+)
Calculated for C₇H₇BrNO (M+H⁺): 199.97110
Found: 200.0

¹H NMR (400 MHz, DMSO-d₆, 25° C.)
δ8.66 (dd, J=4.6, 1.3 Hz, 1H), 8.22 (dd, J=8.2, 1.3 Hz, 1H), 7.52 (dd, J=8.2, 4.6 Hz, 1H), 2.61 (s, 3H).

### Part 2—Synthesis of Compound 4a—(S)-1-(3-bromopyridin-2 yl)ethan-1-ol

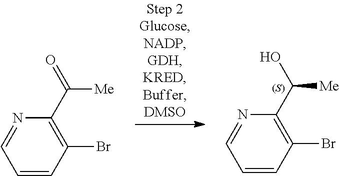

To a reactor was charged potassium phosphate buffer (0.2 M, pH=6-8-7.2, 2,000 L, 10 V), glucose (594 kg, 3,297 mol, 3.3 equiv), GDH (4 kg, 2% w/w), NADP (2 kg, 1% w/w), KRED (2 kg, 1% w/w), and a solution of 1-(3-bromopyridin-2-yl)ethan-1-one (Compound 4) (200 kg, 999.83 mol, 1 equiv) in DMSO (200 L, 1 V) at 25-30° C. Note: the pH was maintained at 6-5-7 as needed, using 2 M aqueous NaOH. The reaction mixture was maintained at 28-32° C. for 6 hours at which point HPLC analysis showed reaction completion.

To the reaction mixture was charged diatomaceous earth (40 kg, 20% w/w) and MTBE (800 L, 4 V). The resulting mixture was filtered, and the cake was washed with MTBE (200 L, 1 V). The resulting phases were separated, and the aqueous phase extracted again with MTBE (500 Lx 3, 2.5 V×3). The combined organic phases were washed with brine (100 L, 0.5 V) and concentrated (45-55° C., −0.08 MPa) to afford (S)-1-(3-bromopyridin-2-yl)ethan-1-ol (Compound 4a) (204 kg, 97.8% a/a purity, 89.6% w/w assay, 90% yield).

### Part 3—Alternative Synthesis of Compound 4a—(S)-1-(3-bromopyridin-2 yl)ethan-1-ol

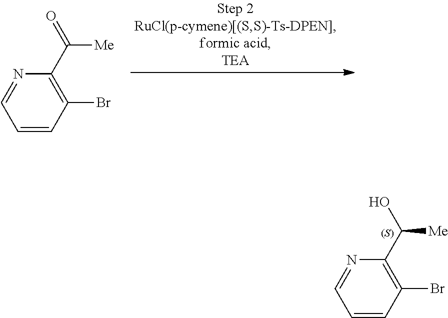

To a reactor was charged triethylamine (47 kg, 464.46 mol, 2.8 equiv). This was cooled to, and maintained at, 0-10° C. To this was charged formic acid (19 kg, 412.82 mol, 2.5 equiv) and RuCl(p-cymene)[(S,S)-Ts-DPEN] (0.55 kg, 864.49 mmol, 0.005 equiv). The resulting mixture was heated to, and maintained at, 30-35° C. To this was charged 1-(3-bromopyridin-2-yl)ethan-1-one (Compound 4) (36.7 kg, 165.12 mol, 1 equiv), rinsing the charging port with additional triethylamine (2 kg, 19.76 mol, 0.12 equiv). The reaction mixture temperature was maintained at 30-35° C. for 6 hours at which point HPLC analysis showed reaction completion.

The reaction mixture was concentrated (30-35° C.) to remove triethylamine. To the resulting mixture was charged water (170 kg) and EtOAc (310 kg) at 15-25° C. The phases were separated, and the aqueous phases extracted with EtOAc (160 kg×2). The combined organic phases were washed with brine (158 kg×2), dried over anhydrous Na₂SO₄, and filtered, washing the spent drying agent cake with EtOAc (40 kg). The combined filtrates were cooled to, and maintained at, 0-10° C. and to this was charged 35% w/w HCl in MeOH (55 kg, 3.2 equiv). The resulting mixture was maintained at 0-10° C. for 12 hours before being filtered, washing the product with EtOAc (40 kg). The product was dissolved in water (66 kg) and EtOAc (170 kg) and the resulting solution was cooled to, and maintained at, 5-15° C. To this was charged a solution of NaHCO₃ (33 kg) in water (170 kg). The phases were separated, and the aqueous phases extracted with EtOAc (170 kg×3). The combined organic phases were washed with brine (158 kg×2), dried over anhydrous Na₂SO₄, and filtered, washing the spent drying agent cake with EtOAc (120 kg). The filtrate was concentrated (40-45° C.) to afford (S)-1-(3-bromopyridin-2-yl)ethan-1-ol (Compound 4a) (30.0 kg, >99.9% a/a purity, 95% w/w assay, 86% yield) as a dark brown oil.

TABLE 2
HPLC Method for Part 2 and 3 of Example 1
HPLC method:

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| | Time (min) | A % | B % |
| Gradient: | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector Wavelength: | 210 nm |
| Column Temperature: | 15° C. |
| Retention Times: | 1-(3-bromopyridin-2-yl)ethan-1-one: 8.0 min |
| | (S)-1-(3-bromopyridin-2-yl)ethan-1-ol: 5.6 min |

LRMS (ESI+)
Calculated for C₇H₉BrNO (M+H⁺): 201.98675
Found: 202.0

¹H NMR (400 MHz, DMSO-d₆, 25° C.)
δ8.56 (dd, J=4.6, 1.4 Hz, 1H), 8.02 (dd, J=8.0, 1.4 Hz, 1H), 7.26 (dd, J=8.0, 4.6 Hz, 1H), 5.13-5.04 (m, 2H), 1.37 (d, J=6.0 Hz, 3H).

### Part 4—Synthesis of Compound 4b—(S)-3-bromo-2-(1-methoxyethyl)pyridine

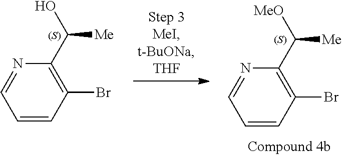

To a reactor was charged THF (2,025 L, 5 V) and t-BuONa (231 kg, 2,404 mol, 1.2 equiv). The resulting mixture was cooled to, and maintained at, 0-10° C. To this was charged a solution of (S)-1-(3-bromopyridin-2-yl)ethan-1-ol (405 kg, 2,004 mol, 1 equiv) in THF (800 L, 2 V) and Mel (340 kg, 2,395 mol, 1.2 equiv). The resulting reaction mixture was maintained at 0-10° C. for 16 hours at which point HPLC analysis showed reaction completion.

To the reaction mixture was charged 7.5% w/w aqueous NH₃ (520 L, 1.3 V) at 0-10° C. and MTBE (1,200 L, 3 V). The phases were separated, and the aqueous layer extracted with MTBE (1,200 L, 3 V). The combined organic phases were washed with brine (200 L, 0.5 V) and concentrated (50-60° C., −0.08 MPa) to afford crude (S)-3-bromo-2-(1-methoxyethyl)pyridine (Compound 4b). The crude (S)-3-bromo-2-(1-methoxyethyl)pyridine was distilled (120° C., 600 Pa) to afford (S)-3-bromo-2-(1-methoxyethyl)pyridine (445 kg, 99.3% a/a purity, 90.2% w/w assay, 93% yield) as a colorless solid (solidified after packaging).

TABLE 3
HPLC Method for Part 4 of Example 1
HPLC method:

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| | Time (min) | A % | B % |
| Gradient: | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector | 210 nm |
| Wavelength: | |
| Column Temperature: | 15° C. |
| Retention Times: | (S)-1-(3-bromopyridin-2-yl)ethan-1-ol: 5.6 min |
| | (S)-3-bromo-2-(1-methoxyethyl)pyridine: 7.8 min |

LRMS (ESI+)
Calculated for C₈H₁₁BrNO (M+H⁺): 216.00240
Found: 216.00

¹H NMR (400 MHz, CDCl₃, 25° C.)
δ8.61 (d, J=3.2 Hz, 1H), 7.83(q, J=1.6, 6.8 Hz, 1H), 7.08(q, J=3.6, 4.8 Hz, 1H), 4.92(q, J=6.4 Hz, 1H), 3.31 (s, 3H), 1.48 (d, J=6.8 Hz, 3H).

### Example 2. Synthetic Procedure for 3,3-dimethyldihydro-2H-pyran-2,6(3H)-dione

Detailed below is a general synthetic procedure for 3,3-dimethyldihydro-2H-pyran-2,6(3H)-dione.

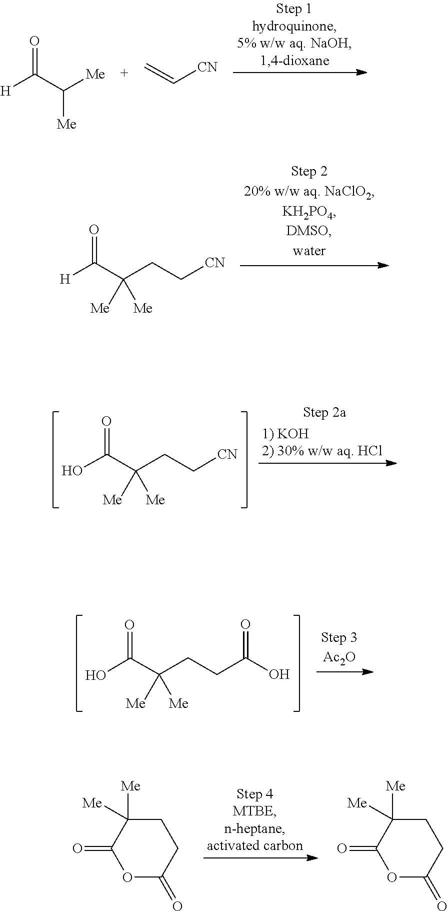

### Part 1—Synthesis of 4,4-dimethyl-5-oxopentanenitrile

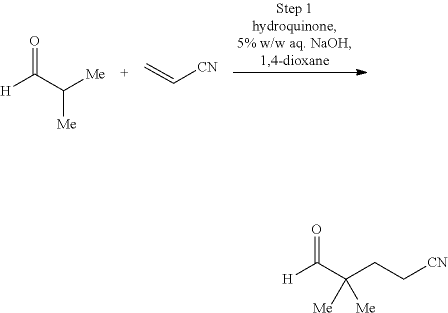

To a reactor was charged 1,4-dioxane (1,552 L, 5 V), hydroquinone (1.55 kg, 14.1 mol, 0.0033 equiv), and 5% w/w aqueous NaOH (341.4 kg, 426.78 mol, 0.1 equiv). The resulting mixture was heated to, and maintained at, 70-75° C. To this was charged isobutyraldehyde (310.6 kg, 4,307.3 mol, 1 equiv) and acrylonitrile (2) (285.7 kg, 5,384.5 mol, 1.25 equiv) over 8 hours. The reaction mixture was maintained at 70-75° C. for 8 hours at which point GC analysis showed reaction completion.

The reaction mixture was then cooled to, and maintained at, 20-25° C. The pH was adjusted to 5-6 with 3.5% w/w aqueous HCl (required 172.5 kg) and concentrated (45° C., −0.03 atm) until no organic solvent was distilled. The remaining residue was cooled to, and maintained at, 20-25° C. To this was charged DCM (1,552 L, 5 V) and water (620 L, 2 V). The phases were separated, and the organic phase concentrated (45° C., −0.03 atm) until no solvent was distilled to afford crude 4,4-dimethyl-5-oxopentanenitrile as a brown oil (626.6 kg, 70.8% a/a purity, 43.5% w/w assay, 51% yield).

TABLE 4
GC Method for Part 1 of Example 2
GC method:

| Column | HP-5: 30 m*0.32 mm*0.25 μm | | | |
|---|---|---|---|---|
| Detector | Temperature | 260° C. | | |
| parameters | Flow H2 | 40 mL/min | | |
| | Air flow | 300 mL/min | | |
| | Makeup (N2) | 25 mL/min | | |
| Injector | Temperature | 280° C. | | |
| parameters | Split ratio | 30:1 | | |
| Carrier gas | N2 | | | |
| | Flow | 2.5001 mL/min | | |
| | Mode | Constant Flow | | |
| | Time | Rate | Temperature | |
| | [min] | [° C./min] | [° C.] | Hold [min] |
| Oven parameters | 0.0 | — | 40 | 3 |
| | | 20 | 260 | 3 |

| | |
|---|---|
| Retention time | isobutyraldehyde: 1.447 min |
| | 4,4-dimethyl-5-oxopentanenitrile: 7.043 min |

LRMS (ESI+)
Calculated for C₇H₁₂NO (M+H): 126.09189
Found: 126.0

¹H NMR (400 MHz, CDCl₃, 25° C.)
δ9.37 (s, 1H), 2.30-2.19 (m, 2H), 1.88-1.77 (m, 2H), 1.06 (s, 6H).

### Part 2—Synthesis of Crude 3,3-dimethyldihydro-2H-pyran-2,6(3H)-dione

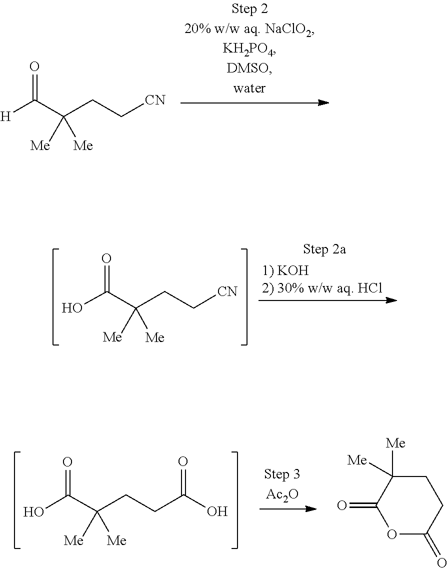

To a reactor was charged water (1,115 kg, 5 V), KH₂PO₄ (13.8 kg, 101.4 mol, 0.057 equiv), DMSO (164.0 kg, 2,099.1 mol. 1.2 equiv), and crude 4,4-dimethyl-5-oxopentanenitrile (455.3 kg, 49.0% w/w assay, 1,782.3 mol, 1 equiv). The resulting mixture was cooled to, and maintained at, 10-20° C. To this was charged 20% w/w aqueous NaClO₂ (1,185.0 kg, 2,620.5 mol, 1.5 equiv) over 20 hours. The reaction mixture was then maintained at a temperature of 10-20° C. for 1 hour at which point GC analysis showed reaction completion. This afforded crude 4-cyano-2,2-dimethylbutanoic acid which was used directly in the next step.

To the mixture of crude 4-cyano-2,2-dimethylbutanoic acid was charged KOH (361.5 kg, 6,442.7 mol, 3.6 equiv). The resulting mixture was extracted with MTBE (800 kg×2, 4.9 V×2). The aqueous phase was then heated to, and maintained at, 90-100° C. for 15 hours at which point GC analysis showed reaction completion.

The reaction mixture was cooled to, and maintained at, 15-25° C. The pH was adjusted to 1-2 with 30% w/w aqueous HCl (required 1,058 kg, 4.9 equiv). The resulting mixture was extracted with MTBE (1,058 kg×2, 6.4 V×2). The combined organic phases were washed with 5% w/w aqueous NaCl (378 kg×2, 1.7 V×2) and concentrated (40-45° C., −0.03 atm) to 670 L (3 V) affording crude 2,2-dimethylpentanedioic acid which was used directly in the next step.

To the mixture of crude 2,2-dimethylpentanedioic acid was charged Ac₂O (614.6 kg, 6,020.1 mol, 3.4 equiv) at 40-45° C. The resulting mixture was concentrated (40-45° C., −0.03 atm) to remove MTBE. The reaction mixture was heated to, and maintained at, 80-85° C. for 2 hours at which point GC analysis showed reaction completion.

The reaction mixture was then concentrated (70-75° C., −0.03 atm) to remove AcOH and Ac₂O until no solvent was distilled. This afforded crude 3,3-dimethyldihydro-2H-pyran-2,6(3H)-dione (390.5 kg, 86.3% a/a purity, 69.3% w/w assay, 107% crude yield) which was used directly in the next step.

### Part 3—Synthesis of 3,3-dimethyldihydro-2H-pyran-2, 6(3H)-dione

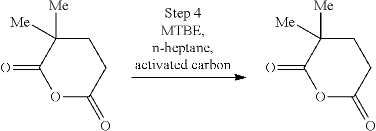 [SMILES: CC1(C)CCC(=O)OC1=O ; CC1(C)CCC(=O)OC1=O]

To a reactor was charged n-heptane (574.0 kg, 1.86 V for crude weight). This was cooled to, and maintained at, −10 to −5° C. To this was charged a solution of crude 3,3-dimethyldihydro-2H-pyran-2,6(3H)-dione (454.0 kg, 84.1% w/w assay) in MTBE (667.2, 2 V for crude weight) over 10 hours. The resulting mixture was maintained at −10 to −5° C. for 1.5 hours and then filtered.

The filter cake was dissolved in MTBE (572 kg, 2 V for assay weight). To the resulting solution was charged activated carbon (19.1 kg, 0.05% w/w for assay weight). This was maintained at 15-25° C. for 8 hours. The resulting solution was then filtered, washing the spent carbon cake with MTBE (18 kg, 0.05 V). The filtrate was then added to pre-cooled (−10 to −5° C.) n-heptane (518.4 kg, 2 V for assay weight) over 9 hours. The resulting mixture was maintained at −10 to −5° C. for 2 hours. This was then filtered at to −5° C. The product was dried (25-30° C., −0.03 atm) for 16 hours to afford 3,3-dimethyldihydro-2H-pyran-2,6(3H)-dione (212.0 kg, 100% a/a purity, 98.3% w/w assay, 55% yield) as an off-white solid.

### Part 4—Alternative Synthesis of 3,3-dimethyldihydro-2H-pyran-2,6(3H)-dione

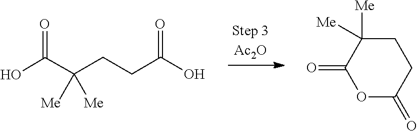 [SMILES: CC(C)(CCC(=O)O)C(=O)O ; CC1(C)CCC(=O)OC1=O]

To a reactor was charged Ac₂O (5.4 L) and 2,2-dimethylpentanedioic acid (2,573 g, 98.8% w/w assay and 506 g, 90.9% w/w assay, 18.74 mol, 1 equiv). The resulting reaction mixture was heated to, and maintained at, 110° C. for 1 hour at which point GC analysis showed reaction completion.

The reaction mixture was concentrated (70° C., ˜0.03 atm) to remove AcOH and Ac₂O until no solvent was distilled. The residue was combined with another batch (2,2-dimethylpentanedioic acid (6,610 g, 90.8% w/w assay)) and distilled (110-120° C., ˜0.005 atm) until no product was distilled. The obtained fraction was triturated with n-heptane (35 L), filtered, and the product dried (25° C., ˜0.005 atm) to afford 3,3-dimethyldihydro-2H-pyren-2,6(3H)-dione (6.66 kg, 99.6% a/a purity, 98.56% w/w assay, 82% yield) as an off-white solid.

TABLE 5
GC Method for Part 2, 3, and 4 of Example 2
GC method:

| Column | HP-5: 30 m*0.32 mm*0.25 um | | | |
|---|---|---|---|---|
| Detector | Temperature | 260° C. | | |
| parameters | Flow H2 | 40 mL/min | | |
| | Air flow | 300 mL/min | | |
| | Makeup (N2) | 25 mL/min | | |
| Injector | Temperature | 280° C. | | |
| parameters | Split ratio | 30:1 | | |
| Carrier gas | N2 | | | |
| | Flow | 2.5 mL/min | | |
| | Mode | Constant Flow | | |
| | Time | Rate | Temperature | Hold |
| | [min] | [° C./min] | [° C.] | [min] |
| Oven | 0.0 | — | 40 | 3 |
| parameters | | 20 | 260 | 3 |

| | |
|---|---|
| Retention | 4,4-dimethyl-5-oxopentanenitrile: 6.931 min |
| time | 4-cyano-2,2-dimethylbutanoic acid: 8.971 min |
| | 2,2-dimethylpentanedioic acid: 9.952 min |
| | 3,3-dimethyldihydro-2H-pyran-2,6(3H)-dione: 8.073 min |

LRMS (ESI+)
Calculated for C₇H₁₁O₃ (M+H⁺): 143.07082
Found: 143.0

¹H NMR (300 MHz, CDCl₃, 25° C.)
δ2.82 (t, J=7.0 Hz, 2H), 1.85 (t, J=7.0 Hz, 2H), 1.35 (s, 6H).

### Part 5—Alternative Synthesis of 2,2-dimethylpentanedioic acid

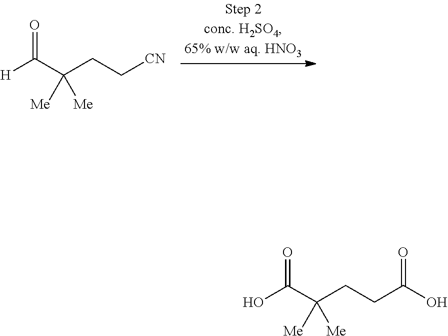 [SMILES: CC(C)(C=O)CCC#N ; CC(C)(CCC(=O)O)C(=O)O]

To a reactor was charged 65% w/w aqueous HNO₃ (3.3 L) and concentrated H₂SO₄ (500 mL) at 25° C. The resulting mixture was heated to, and maintained at, 70-80° C. To this was charged 4,4-dimethyl-5-oxopentanenitrile (2.21 kg, 90.5% w/w assay, 15.98 mol, 1 equiv) in portions over 24 hours. The reaction mixture was then maintained at 70-75° C. for 1 hour at which point GC analysis showed reaction completion.

The reaction mixture was cooled to 25° C. and then charged into ice cold water (10 kg) during which time solid precipitated. The resulting mixture was extracted with MTBE (10 Lx 1 followed by 5 Lx 2). The combined organic phases were washed with water (2 Lx 1), washed with brine (2 Lx 1), dried over anhydrous Na₂SO₄, filtered, and then concentrated (45° C., ˜0.03 atm) until no solvent was distilled. This afforded 2,2-dimethylpentanedioic acid (2.6 kg, 98.8% w/w assay, 100% yield) as a white solid.

TABLE 6
GC Method for Part 5 of Example 2
GC method:

| Column | HP-5: 30 m*0.32 mm*0.25 μm | | | |
|---|---|---|---|---|
| Detector parameters | Temperature | 260° C. | | |
| | Flow H2 | 40 mL/min | | |
| | Air flow | 300 mL/min | | |
| | Makeup (N2) | 25 mL/min | | |
| Injector parameters | Temperature | 280° C. | | |
| | Split ratio | 30:1 | | |
| Carrier gas | N2 | | | |
| | Flow | 2.5 mL/min | | |
| | Mode | Constant Flow | | |
| | Time | Rate | Temperature | Hold |
| | [min] | [° C./min] | [° C.] | [min] |
| Oven parameters | 0.0 | — | 40 | 3 |
| | | 20 | 260 | 3 |

| | |
|---|---|
| Retention time | 4,4-dimethyl-5-oxopentanenitrile: 6.931 min |
| | 2,2-dimethylpentanedioic acid: 9.952 min |

LRMS (ESI−)
Calculated for C₇H₁₁O₄ (M−H⁺): 159.06573
Found: 159.2

¹H NMR (400 MHz, CDCl₃, 25° C.)
δ2.42 (t, J=7.5 Hz, 2H), 1.92 (t, J=7.5 Hz, 2H), 1.23 (s, 6H).

### Example 3. Synthetic Procedure for Compound 5

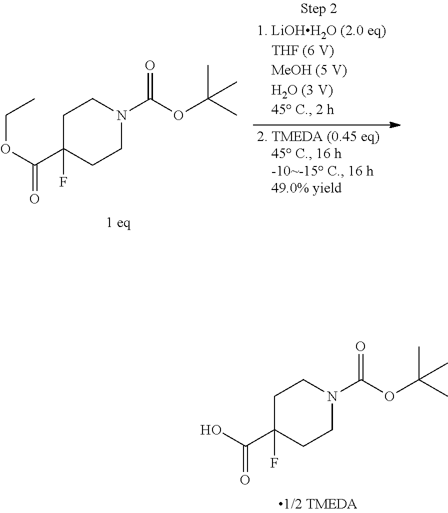 [SMILES: CCOC(=O)C1(F)CCN(C(=O)OC(C)(C)C)CC1 ; CC(C)(C)OC(=O)N1CCC(F)(C(=O)O)CC1]

### Part 1—Compound 5 TMEDA Salt

To a 3000-L glass-lined reactor was added THF (445 L, 6 V), crude 1-tert-butyl 4-ethyl 4-fluoropiperidine-1,4-dicarboxylate (74.0 kg corrected by assay, 268.8 mol, 1.0 eq) and MeOH (370 L, 5 V) at 20±5° C., and the mixture was stirred at 20±5° C. for 15 min. A solution of LiOH·H₂O (22.6 kg, 538.6 mol, 2.0 eq) in H₂O (225 L, 3 V) was added dropwise over 30 min (slightly exothermic) to the reaction mixture at 20±5° C. and the mixture was warmed to 45° C. and stirred for 10 h. HPLC analysis indicated the reaction was complete. The reaction mixture was cooled to 20±5° C. and diluted with H₂O (740 L, 10 V). The mixture was washed successively with IPAc (740 L, 10 V), DCM (740 L×2, 10 V×2), and then partitioned with EtOAc (740 L, 10 V). The pH of the resulting biphasic mixture was adjusted pH to 3 with 7% w/w aqueous HCl (185 kg). The phases were separated, and the lower lean aqueous phase was extracted with EtOAc (740 L, 10 V). The combined organic phases (1398.8 kg) were washed with 5% w/w brine (370 L, 5 V), dried with MgSO₄ (84.6 kg), and filtered. The spent drying agent cake was washed with EtOAc (190.6 kg).

Two batches of rich organic layers (74.0 kg corrected by assay×2) were combined and concentrated under reduced pressure (−0.03 atm) at 45±5° C. to 4 V. TMEDA (28.1 kg, 241.9 mol, 0.45 eq) was added at 45±5° C., and the mixture was stirred at 45±5° C. for 16 h. The mixture was slowly cooled to −10˜−15° C. within 6 h and stirred for an additional 16 h. The slurry was filtered at −10° C., and the product was washed with cold EtOAc (80 L, −10° C.) to afford wet Compound 5½ TMEDA salt. The wet product was triturated with EtOAc/n-heptane (1/1, 170 L, 2 V) at 25° C. for 2 h and filtered. The product was washed with EtOAc/n-heptane (1/1, 42.5 L, 0.5 V) and dried in oven at 45±5° C. under vacuum (˜0.005 atm) (drying criteria: LOD<1.0%) for 12 h to afford Compound 5½ TMEDA salt (80.0 kg, 99.8% a/a purity (TFA method), 99.6% a/a purity (H₃PO₄ method), 98.2% w/w assay, 47.9% assay-corrected yield).

LRMS (ESI−)
Calculated for Calculated for C₁₁H₁₈FNO₄ (M−H⁺): 246.12
Found: 246.2

¹H NMR (300 MHz, d6-DMSO, 24° C.)
δ3.80 (bd, 4H), 2.98 (bs, 4H), 2.78 (s, 4H), 2.42 (s, 12H), 1.07-1/79 (m, 4H), 1.77-1.71 (m, 4H), 1.41 (s, 18H)

### Part 2—Compound 5 DABCO Salt

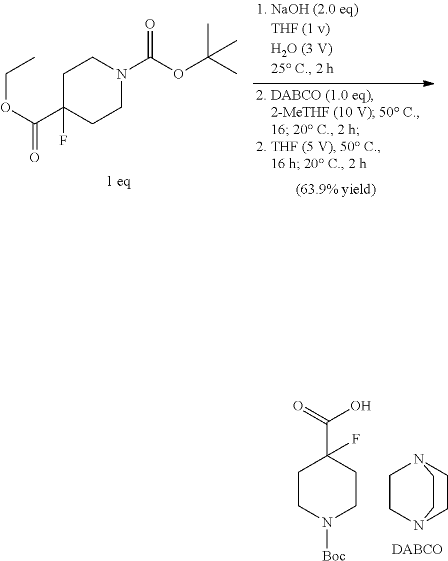

To a 5 L round bottom flask was charged THF (200 mL, 1 V) and crude 1-tert-butyl 4-ethyl 4-fluoropiperidine-1,4-dicarboxylate (164.2 g corrected by assay, 0.669 mmol, 1.0 equiv). To the mixture was charged a solution of NaOH(s) (53.52 g, 1.338 mmol, 2.0 equiv) in water (600 mL, 3 V) at 25±5° C. (inner temperature). The resulting mixture stirred for 2 hrs at 25±5. HPLC analysis indicated the reaction was complete. The reaction mixture was diluted with water (2.00 L, 10 V) and washed twice with 2-MeTHF (1.00 L×2, 5 V×2). The mixture was partitioned with EtOAc (1.00 L, 5 V) and the pH was adjusted to pH 2-3 with 3N HCl aqueous solution. The phases were separated, and the lower lean acidic aqueous layer was extracted with EtOAc (1.00 L, 5 V). The combined organic layers were concentrated under reduced pressure vacuum (−0.1 MPa) at 40±5° C. (jacket temperature) to afford crude Compound 5 as yellow solid (160.0 g, 81.33% a/a purity, 86.8% w/w assay, 94.2% assay-correct yield).

To a 5 L round bottom flask was charged crude Compound 5 (150.0 g (130.2 g corrected by assay, 0.53 mol, 1.0 eq)) and 2-MeTHF (750 mL, 5 V). The mixture was warmed to 50±5° C. and DABCO (56.45 g, 0.53 mol, 1.0 eq) as a solution in 2-MeTHF (750 mL, 5 V) was charged. The mixture was stirred at 50±5° C. for 16 hrs, cooled to 20-25° C., and was stirred for an additional 2 hrs before being filtered. The resulting cake was slurried at 50±5° C. in THF (750 mL, 5 V) and isolated by filtration, before being dried at 40-50° C. under vacuum to afford Compound 5 DABCO salt as a white solid (121.1 g. 99.62% a/a purity, 63.9% yield).

¹H NMR (400 MHz, D2O, 23° C.)
δ3.90 (bd, 2H), 3.13 (s, 12H), 2.96 (t, 2H), 1.97-1.59 (m, 4H), 1.35 (s, 9H)

### Example 4. Synthetic Procedure for Compound 6

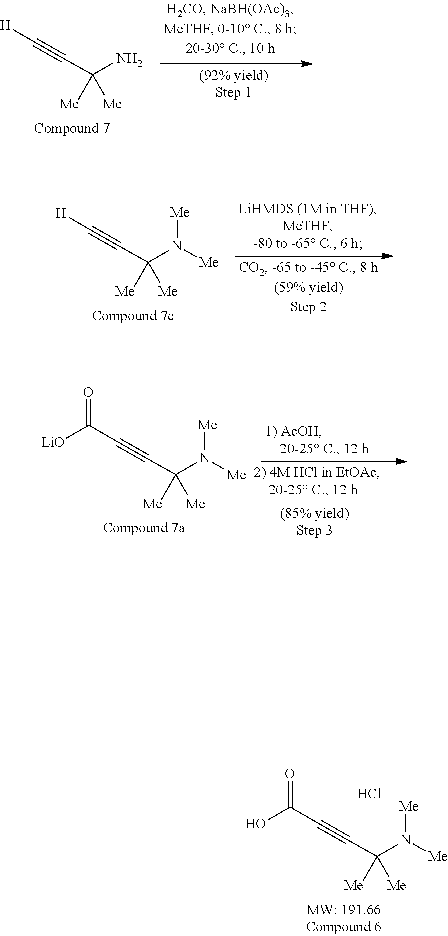

2-MeTHF (568.0 kg, 20 V) and Compound 7 (33.4 kg, 72.2 wt %, 289.3 mol, (assay corrected), 1.0 eq) were charged to a 2000 L glass-lined reactor (Reactor A) under positive nitrogen pressure. The mixture was stirred at 100 RPM for 10 minutes and then was cooled to 0-10° C. CH₂O (37 wt % eq. solution, 163.6 kg, 2016 mol, 7.0 eq.) was charged dropwise to the cold solution at a rate of 1.6 kg/min while maintaining the internal temperature at 0-10° C. The reaction mixture was stirred for 10 minutes at 0-10° C. NaBH(OAc)₃ (188.0 kg, 887 mol, 3.1 eq.) was charged to the reactor, via a sold feeder, in nineteen portions over eight hours while maintaining the temperature at 0-10° C. The reaction mixture was warmed to 20-30° C. over 3 hours and then was stirred for an additional 10 hours. GC analysis indicated the reaction was complete with <1.0 area %, of Compound 7.

The reaction mixture was transferred to a 3000 L glass lined reactor (Reactor B) at 0-20° C. 15 wt % Na₂CO₃(aq) (1960.0 kg, 57.8 w/w.) was charged over 90 minutes to the reactor under agitation (100 RPM) at 0-10° C. The biphasic mixture was warmed to 10-20° C. and was stirred for an additional 20 minutes. The layers were separated; the lean lower aqueous layer was returned to the 3000 L. Reactor B, and the rich upper organic layer was charged to the 2000 L Reactor A. The aqueous layer was extracted twice with 2-MeTHF (284.0 kg, 10 V). The combined organic layers in Reactor A were washed successively with 10 w % NaCl(aq) (334.0 kg×2) and dried over Na₂SO₄ (100.0 kg, 3.0 w/w) with agitation (100 RPM) at 10-20° C. for 3 h. The mixture was filtered under reduced pressure. The spent drying agent was rinsed with 2-MeTHF (57.0 kg, 1.7 w/w). The filtrate was transferred to Reactor A and was cooled to 10-20° C. 4 Å molecular sieves (200.0 kg, 6.0 w/w) were charged slowly at 10-20° C. over three hours to the solution. The mixture was warmed to 20-30° C. and was held at 20-30° C. for 12 h under a slight pressure of N₂ but with only periodic agitation at 30 RPM for one min every hour. The mixture was filtered, and the spent molecular sieves were washed twice with 2-MeTHF (28.0 kg, 2 V). The filtrate was partitioned into eight 200 L HDPE drums with 140 kg of filtrate per drum. 4 Å molecular sieves (10.0 kg, ca. 7 wt % relative to the 2-MeTHF solution) were charged slowly to each drum. The filtrate was held at 20-35° C. for 72 h (12-120 hours). KF assay of the 2-MeTHF solution of Compound 7c met the criteria of </=1000 ppm.

A solution of Compound 7c in 2MeTHF (568.0 kg, 2.68 wt % assay, 1.0 eq) was charged by vacuum to a 1000 L stainless steel reactor under nitrogen pressure. Note an in-line filter was used to isolate the molecular sieves contained in the feed drum. The solution was cooled −80 to −75° C. under agitation (100 RPM). LiHMDS (190.8 kg, 1M in THF, 1.6 eq.) was charged dropwise (−1.5 kg/min) while maintaining the temperature at −80 to −65° C. The reaction mixture was further stirred for six hours at −80 to −65° C. under nitrogen. CO₂(g) (60 kg) was charged to the reaction mixture at a rate of 80-100 g/min, at 65 to −45° C. and until the reaction mixture was saturated. Saturation was determined when a lot of gas was observed in the tail gas by the bubbler. The reaction mixture was further stirred for eight hours at −62° C. to −45° C. Assay of the reaction by HPLC indicated the reaction was complete with <5.0 area % Compound 7c. The reaction mixture was gradually warmed to 20-30° C. over 10-20 hours in order to release the dissolved CO₂ gas. The reaction mixture was transferred to a 2000 L glass lined reactor and concentrated under reduced pressure at 10-20 bar and 40-50° C. to target volume of 50-60 L. n-Heptane (45.0 kg, 66 L) was charged to the reactor, and the mixture was concentrated again to 50-60 L. The isolation process was repeated two more times with n-heptane (2×45.0, 66 L) to a final volume of 50-60 L. EtOAc (107.3 kg, 120 L) was charged to the reactor. The mixture was stirred at 20-30° C. for three hours to dissolve the solids. n-heptane (246.0 kg, 360 L) was charged to the reactor. The slurry was further stirred at 20-30° C. for three hours and then filtered. The resulting solids were washed twice with n-heptane (2×45.0 kg, 66 L) and dried under reduced pressure (10-15 mbar) at 40-45° C. for 20 h to afford 24.7 kg of Compound 7a (lithium salt) with 89% HPLC purity and 53.8 wt % assay by qNMR was obtained in a 61.3% yield as a white solid.

AcOH (630.0 kg, 5.24 w/w) was charged to a 3000 L glass-lined reactor. Compound 7a (120.0 kg) was charged in 10 portions (10-15 kg per portion) over 90 minutes, while maintaining the temperature at 20-25° C. The mixture was stirred at 20-25° C. for six hours to achieve a clear solution. EtOAc (2160.0 kg, 18 w/w) was charged to the solution over 30 minutes. The mixture was stirred for 12 hours at 20-25° C. The resulting solids were isolated by centrifuge, washed twice with EtOAc (2×270.0 kg, 2.25 w/w) to afford 116.2 kg of wet product Compound 7b AcOH salt.

The wet product (116.2 kg) and EtOAc (2160.0 kg, 18 w/w) were charged to a 5000 L glass-lined reactor. To the resulting suspension was charged 4 M HCl in EtOAc (240 kg, 2 w/w.) over 25 min (ca. 10 kg/min) at 20-25° C. The resulting suspension was stirred at 20-25° C. for twelve hours. The resulting solids were isolated by centrifuge, washed twice with EtOAc (2×130.0 kg, 1.1 w/w), and dried under reduced pressure at 35-40° C. for 40 hours to afford 63.5 kg of Compound 6 with 96.2% HPLC purity, 1757 ppm AcOH residual by HS-GC and 97.7 wt % assay by qNMR was obtained in an 85.3% corrected yield as a white solid.

LRMS (ESI+)
Calculated for C₈H₁ₕNO₂ (M+H⁺): 156.09
Found: 156.1

¹H NMR (400 MHz, d6-DMSO)
δ2.78 (s, 6H), 1.70 (s, 6H)

### Example 5. Synthetic Procedure for Compound 11b

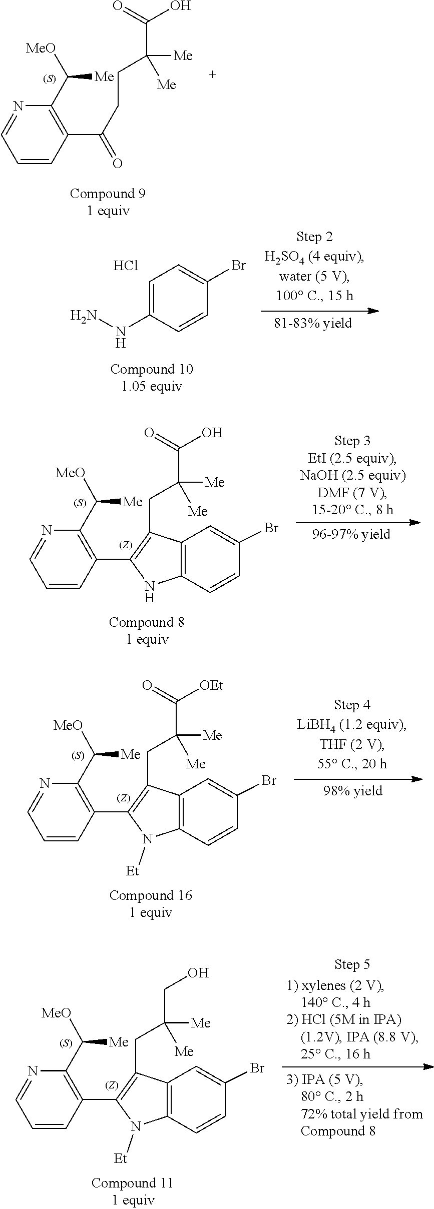

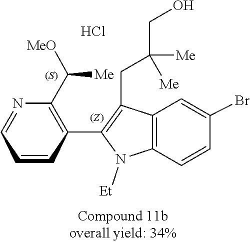

### Part 1—Synthesis of Compound 9—(S)-5-(2-(1-methoxyethyl)pyridin-3-yl)-2,2-dimethyl-5-oxopentanoic acid

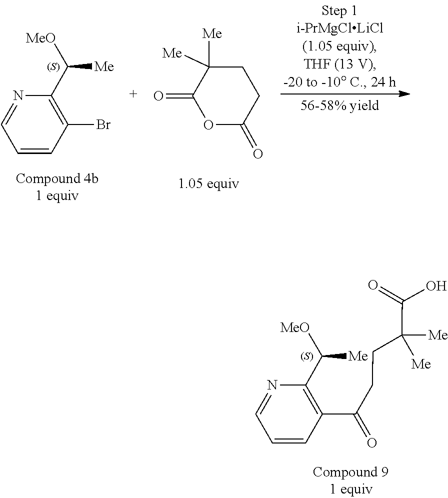

To a 3,000 L Hastelloy lined jacketed reactor was charged THF (1,200 L, 10.0× by volume, 70 L/min) and (S)-3-bromo-2-(1-methoxyethyl)pyridine (Compound 4b, 120 kg, 20 kg/min) at 15-25° C. under N₂. The resulting mixture was cooled to −15 to −10° C.

To the reactor was charged i-PrMgCl·LiCl (448 L, 3.74× by volume, 1.05 eq) dropwise at −25˜−15° C. The reaction mixture was agitated at −15˜−5° C. for 1 h. under N₂.

To the reactor was charged a solution of 3,3-dimethyldihydro-2H-pyran-2,6(3H)-dione (82.9 kg, 0.69× by weight, 1.05 eq) in THF (360 L, 3.00× by volume) under −15˜−5° C. under N₂. The reaction mixture was agitated at −8˜−12° C. for 12 h.

The reaction mixture was quenched by charging water (360 L, 3.00× by volume) at 0-10° C. under N₂ and then was concentrated to 500-600 L. To the concentrated mixture was charged a mixture of AcOH (60.0 L, 0.50× by volume) and water (1,200 L, 10.0× by volume). The biphasic mixture was extracted twice with EtOAc (1,200 L, 10.0× by volume). The combined organic phases were concentrated to 400-500 L.

n-Heptane (1,205 L, 3.00× by volume) was charged to the concentrated solution and the resulting mixture was concentrated to about 400˜500 L. This unit operation was repeated two more times. A solution of MTBE: n-heptane=1:1 (1,205 L, 3.00× by volume) was charged to the concentrated solution. The resulting slurry was agitated at 15˜20° C. under N for 16 h. The solids were isolated by filtration and dried at 35˜40° C. for 12˜24 h under N₂ to obtain Compound 9 as an off-white solid (57.5% isolated yield, 99.2% w/w Assay, 100% ee, 99.5% purity).

¹H NMR (400 MHz, DMSO-d₆, 25° C.)
δ12.20 (s, 1H), 8.60 (d, J=3.2 Hz, 1H), 7.89 (dd, J=15.6, 1.2 Hz, 1H) 7.39 (dd, J=7.6, 4.8 Hz, 1H), 4.55 (q, J=6.4 Hz, 1H), 3.11 (s, 3H), 2.80 (dd, J=16.0, 8.0 Hz, 2H), 1.80 (dd, J=16.0, 8.0 Hz, 2H), 1.42 (d, J=6.4 Hz, 3H), 1.12 (s, 6H)

### Part 2—Synthesis of Compound 8—(S)-3-(5-bromo-2-(2-(1-methoxyethyl)pyridin-3-yl)-1H-indol-3-yl)-2,2-dimethylpropanoic acid

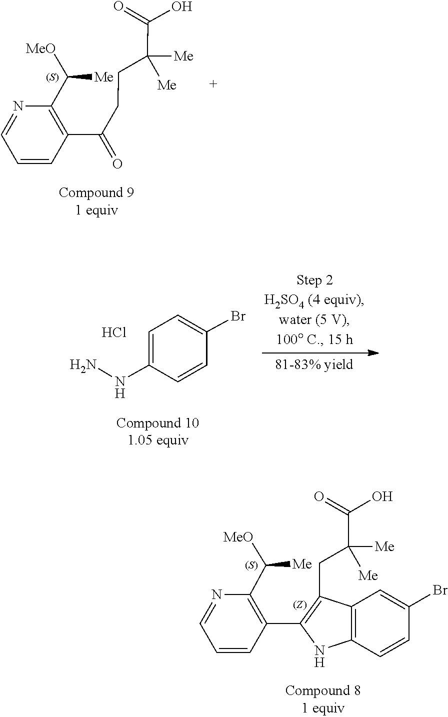

To a 5,000 L glass-lined reactor was charged water (1,565 L, 5.00× by volume), H₂SO₄ (440 kg, 1.40× by weight, 4.00 eq), Compound 9 (313 kg, 1 eq.) and Compound 10 (250 kg, 0.80× by weight, 1.00 eq) at 10-20° C. under N₂. The mixture was warmed to 95-100° C. and agitated for 18 h. The reaction mixture was cooled to 50-60° C. and neutralized to pH 4-5 by adding 20% wt. aqueous NaOH solution (1,800 L) under N₂. Compound 8 was precipitated by further adjusting the pH to 6.2-6.8 using aqueous K₂HPO₄ solution (about 65 L). The solids were isolated by centrifugation, rinsed with water (1,290 L, 5.00× by volume), and dried under N₂ at 45˜50° C. for 24 h to obtain Compound 8 (456 kg, 80.6% assay-corrected yield, 85.0% area purity) as a brown solid.

### Analytical Data for Compound 8

¹H NMR (400 MHz, DMSO-d₆, 25° C.)
δ12.14 (bs, 1H), 11.42 (s, 1H), 8.71 (dd, J=4.4, 1.2 Hz, 1H), 7.45 (dd, J=8.0, 4.8 Hz, 1H), 7.31 (d, J=8.8 Hz, 1H) 7.21 (d, J=8.4 Hz, 1H), 4.18 (q, J=6.4 Hz, 1H), 2.93-2.77 (m, 5H), 1.36 (d, J=6.0 Hz, 3H), 0.88 (d, J=6.4 Hz, 6H)

### Part 3 Synthesis of Compound 16—ethyl (S)-3-(5-bromo-1-ethyl-2-(2-(1-methoxyethyl)pyridin-3-yl)-1H-indol-3-yl)-2,2-dimethylpropanoate

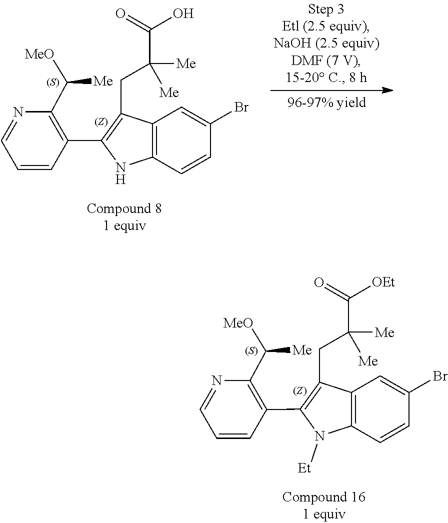

To a 5,000 L glass-lined reactor was charged DMF (973 L, 7.00× by volume) and Compound 8 (139 kg, 1 eq.) at 10-20° C. under N₂. The mixture was agitated at ambient temperature to afford a clear solution and was then cooled to 0-5° C.

To the mixture was charged NaOH (32.2 kg, 0.24× by weight, 2.50 eq) and Etl (126 kg, 0.91× by weight, 2.50 eq). The reaction mixture was warmed to 15-20° C. and agitated for 6 h.

The reaction was quenched with water (1,390 L, 10.0× by volume) at 0-20° C. The resulting mixture was extracted with MTBE two times (5-10× by volume). The combined organic layers were concentrated at 35-45° C. The concentrated mixture was diluted with THF (278 L, 2.00× by volume) and further concentrated to about 2 v (about 150 L) under reduced pressure at 35-45° C. The previous unit operation was repeated until the MTBE content in THF solution was below 1% w/w. The resulting THF solution of Compound 16, as a 1:1 mixture of diastereomers, was used directly in the next step (385 kg, 33.5% w/w HPLC assay, 96.3% isolated yield, assay: 129 kg)

### Part 4—Synthesis of Compound 11—(S)-3-(5-bromo-1-ethyl-2-(2-(1-methoxyethyl)pyridin-3-yl)-1H-indol-3-yl)-2,2-dimethylpropan-1-ol

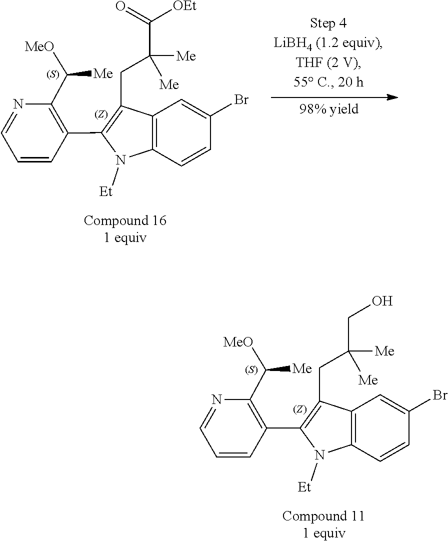

To a 1,000 L glass-lined reactor was charged the THF solution of Compound 16 (661.8 kg, assay of Compound 16: 205.3 kg) at 15˜20° C. under N₂. To the mixture was charged LiBH₄ (2M in THF, 1.20 eq) at 50˜60° C. and then was agitated at 50˜60° C. under N₂ for 12 h.

The reaction was quenched into 0.50 M HCl(aq) (1,455 L, 7.08× by volume). The pH of the mixture was adjusted to pH 4-5 with 0.50 M HCl (aq). The resulting mixture was extracted with EtOAc (1,810 L, 8.80× by volume). The phases were separated. The organic layer was washed with brine (743 L, 3.61× by volume) and concentrated to 1300 L to obtain an EtOAc solution of Compound 11a and 11b as a 1:1 mixture of diastereomers (1,484 kg EtOAc solution, 93.12% area purity, 97.6% assay-corrected yield, 11a+11 b assay: 183 kg).

### Part 5—Isolation of 11b from Compound 11a and 11b Mixture

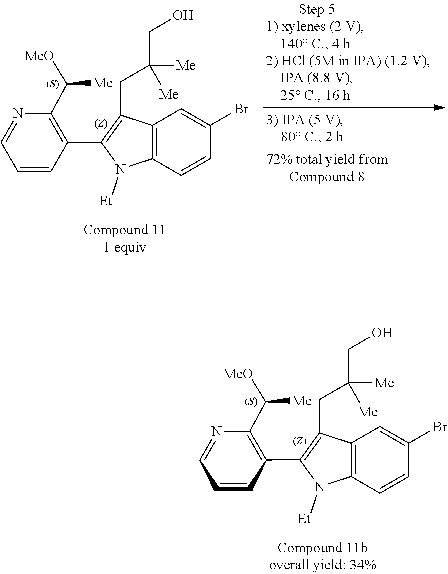

The EtOAc solution of Compound 11a and 11b was concentrated to 1.2-1.5 V in a 5,000 L glass-lined reactor, to which was charged xylene (2.29× by volume). The mixture was agitated at 90-100° C. for 2 h to evaporate residual EtOAc, then heated to 135-140° C. for 4 h to equilibrate the mixture of diastereomers to a ratio of less than 1:1.6 11a: 11 b). The reaction mixture was then cooled to 20-25° C., concentrated to 1.2-1.5 V, and diluted with IPA (3.87× by volume) at 40-45° C. The solution was further cooled to 20-25° C., diluted with additional IPA (4.77× by volume) and treated with a solution of HCl in IPA (5 mol/L, 1.32× by volume). The resulting suspension was agitated at 20-25° C. for 16-24 h and then filtered. The resulting wet cake was dissolved in IPA (5.00× by volume) with agitation at 70-75° C. for 4 h and then at 20-25° C. for 10 h. The solids were isolated by filtration and dried at 35-40° C. under N₂ for 24 h to afford the 1^{st} crop of compound 11 b (388 kg, 99.0% area purity, 99.7% chiral purity, 46% yield) as light-yellow solid.

The product-rich filtrates were combined and concentrated to near dryness. The resulting brown oil was purified by silica gel chromatography (column height: 250 mm, diameter: 100 mm, 100-200 mesh silica gel, n-heptane/ethyl acetate=from 5/1 to 0/1) to afford product-rich fractions of Compound 11a and 11 b mixture. This mixture was subject to another round of equilibration as described above to afford Compound 11b (151 kg, 98.7% area purity, 99.7% chiral purity, 15% yield) as light-yellow solid.

¹H NMR (400 MHz, DMSO-d₆, 25° C.)
δ8.93 (d, J=4.0 Hz, 1H), 8.35 (d, J=7.6 Hz, 1H), 7.89-7.92 (m, 2H), 7.52 (d, J=8.8 Hz, 1H), 7.32 (dd, J=8.4, 1.6 Hz, 1H), 4.02-4.15 (m, 2H), 3.75-3.85 (m, 1H), 2.93-3.04 (m, 5H), 2.67 (d, J=14 Hz, 1H), 2.12 (d, J=14 Hz, 1H), 1.44 (d, J=6 Hz, 3H), 1.11 (t, J=6.8 Hz, 3H), 0.64 (s, 3H), 0.60 (s, 3H)

### Example 6. Alternative Synthesis of Compound 11b

This Example describes an alternative synthesis of Compound 11b. This method utilizes an acid chloride derivative of 3,3-dimethyldihydro-2H-pyran-2,6(3H)-dione to prepare compound 9. Additional methods via Compound 22 improve the initial ratio of desired atropisomer Compound 11b to undesired Compound 11a. This method may have the benefit of utilizing more readily available and cost-effective substrates.

An advantage of utilizing a stepwise alkylation is higher diastereoselectivity of the indole alkylation, affording the desired Compound 11b over the undesired Compound 11a in a ratio of greater than 5:1.

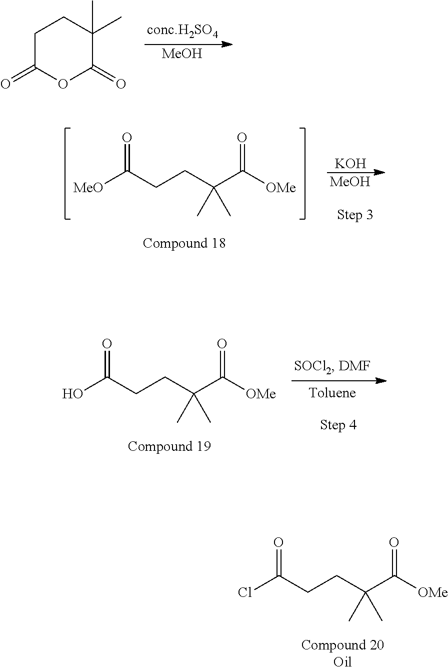

### Preparation of Compound 20

2,2-dimethyl glutaric anhydride (500 g), methanol (8 V) and sulfuric acid (0.13 equiv.) were charged to a reaction vessel. The mixture was stirred at 55° C. for 24 hours. The solvent was distilled under vacuum at 50° C. The residue was partitioned with water (3 V) and MTBE (7 V). The organic layer was washed with water and brine and distilled under vacuum to afford diester Compound 18 as an oil. The crude di-ester, methanol (8 V) and potassium hydroxide (1.2 equiv.) were stirred at room temperature for 14 hours, and heated to reflux for an additional two hours. The mixture was cooled to room temperature, concentrated under reduced pressure, acidified with 1M HCl(aq) and partitioned with brine and MTBE. The organic phase was concentrated to afford carboxylic acid Compound 19 as a solid. Compound 19, toluene (5 V) and DMF (1 wt %), were charged to a reactor. Thionyl chloride (1.5 equiv.) was charged slowly while maintaining the temperature at 0-10° C. The reaction mixture was stirred at room temperature for 14 hours. The solvent was removed vacuum distillation at 50-55° C., and the product was isolated by high vacuum distillation to afford Compound 20 as an oil.

### Preparation of Compound 21

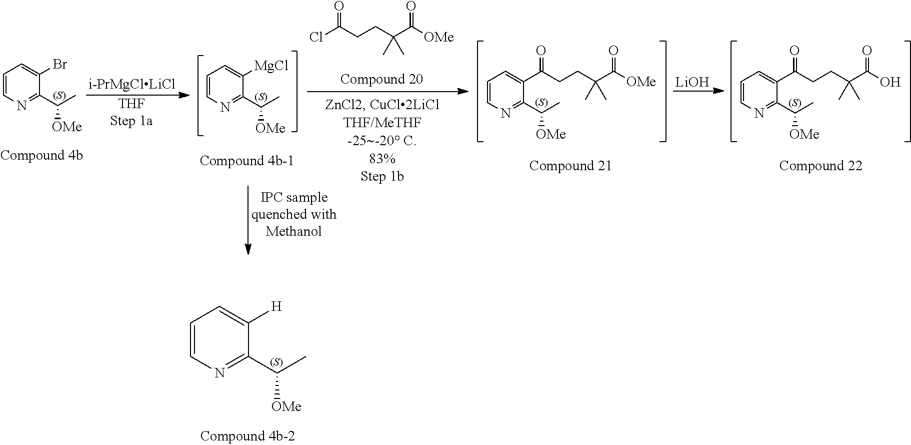

Step 1a: To a reactor R1 was charged THF (100 mL, 1.0 vol.) under nitrogen protection. 1.3 M i-PrMgCl·LiCl THF solution (374 mL, 1.05 eq.) was added into the mixture at 20-30° C. The temperature was adjusted to between −10 and 0° C. Compound 4b (100 g, 1.0 eq.) in THF (200 mL, 2 vol.) was added dropwise to the reaction mixture at −10-0° C. The mixture was stirred at −10 to 0° C. for 2 h followed by sampling for IPC (HPLC purity: 95.0 A % of Compound 4b-2 and 0.2 A % of Compound 4b).

Step 1b: 2-MeTHF (300 mL, 3 vol.) and ZnCl₂ (72.3 g, 1.15 eq.) were charged to reactor R2 under nitrogen protection. The temperature was adjusted to between −10 and 0° C. The reaction mixture from R1 was added dropwise to R2 at −10 to 0° C. over 1 h. THF (50 mL, 0.5 vol.), CuCl (4.6 g, 0.1 eq.) and LiCl (3.9 g, 0.2 eq.) were charged into reactor R3 and stirred for 1 h at 20 to 30° C. The reaction mixture in R3 was dropwise added to R2 at −10 to 0° C. Compound 20 (107 g, 1.2 eq.) was dropwise added into R2 at −10 to 0° C. The mixture was stirred at −10 to 0° C. for 16 h followed by sampling for IPC (HPLC purity: 92.7 A % of Compound 21 and 0.6 A % of Compound 4b-2). The reaction mixture was warmed to 25-30° C. Toluene (500 mL, 5 vol.), AcOH (83.3 g, 3.0 eq.) and H₂O (300 mL, 3.0 vol.) was added to the reaction mixture and stirred for 0.5 h. The organic phase was separated, and the aqueous phase was extracted with toluene (300 mL, 3 vol.). The combined organic phase was washed with 10% ammonia hydroxide (300 mL×2, 3 vol.×2). The organic phase was concentrated under vacuum to 1-1.5 vol. 158 g of Compound 21 was obtained as brown oil with 96.2% HPLC purity and 89% uncorrected yield.

Step 1c: Water (5 vol) was added to the crude and stirred for 15 min. The reaction was cooled to 5±5° C. Lithium hydroxide (2 eq) was slowly added at 5±5° C. The temperature was raised to 25±5° C. and stirred for 14-16 hrs. The progress of the reaction was monitored by HPLC (Limit: Compound 21: NMT 1.0%), After reaction completion, the reaction was washed with MTBE (2×5 vol), the pH was adjusted to 5-6 with 50% acetic acid solution (1 vol) (solid precipitation occurs). The resulting mixture was stirred for 3-4 h, the solids were filtered and washed with water (5 vol) followed by with heptane (5 vol). The product was dried under vacuum at 50° C.
Purity: NLT: 95%, Yields range: 65-70%

### Preparation of Compound 8 Hemisulfate Salt

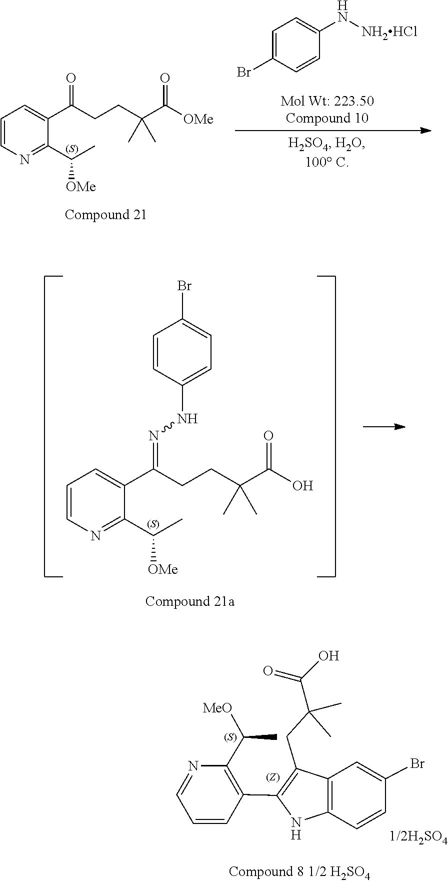

This procedure reduces the des-bromo impurity and allows for the efficient flow chemistry for recycling Compound ha/Compound 11b mixture (vide infra).

To a reactor R1 was charged H₂O (50 mL, 2.5 vol.) and conc. H₂SO₄ (13.4 g, 1.0 eq.) under PGP-151,C₃ nitrogen protection. Compound 21(20.0 g. 1.0 eq.) and Compound 10 (16.0 g, 1.05 eq.) were added into the mixture at 20-30° C. The temperature was adjusted to 90-100° C. The mixture was stirred at 90-100° C. for 48 h followed by sampling for IPC (HPLC purity: 84.2 A % of Compound 8, 0.2 A % of Compound 21 and 1.1 A % of Compound 21a). The mixture was cooled to 60-05° C., and H₂O (110 mL, 5.5 vol.) was added into the mixture at 60-65° C. Then NaOH (5.5 g, 2.0 eq.) was added into the mixture at 60-65° C., the mixture was stirred for 2 h at 60-65° C. The mixture was cooled to 20-25° C. and stirred for 2 h. The mixture was filtered, and the filter cake was rinsed with H₂O (40 mL, 2.0 vol.). The wet filter cake was dried at 50-55° C. under reduced pressure (−0.95 Mpa) for 14 h. 26.9 g of Compound 8½SO₄ was obtained as an off-yellow solid with 96.2% HPLC purity and 78% corrected yield.

### Alternative Experimental Procedure for Preparing Compound 8 Hemisulfate Salt

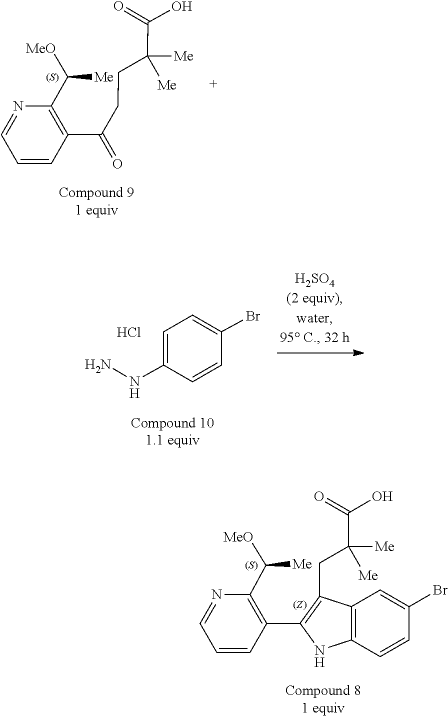

Sulfuric acid (238 kg, 2.0 equiv.) was add dropwise to water (807.7 L) under N₂ while the temperature was maintained at 20° C.±20° C. Compound 9 (340 kg, assay corrected) and Compound 10 (299.18 kg, 1.1 equiv.) were charged at 20±10° C. The reaction mixture was stirred at 70-75° C. for no less than 30 minutes and further stirred at 95±10° C. for 32 hours. Reaction monitoring by HPLC showed the reaction was complete with </=1% of Compound 9 detected. The reaction mixture was cooled to 60-65° C. Water (1871.5 kg) and 30 wt % NaOH(aq) (298.5 kg, 1.9 equiv.) were charged, and the mixture was stirred for no less than five hours. The slurry was cooled to 25±5° C. over four hours, further stirred at 25±5° C. for two hours, and filtered. The resulting solids were washed with water (1020 kg) to afford 538.95 kg of a wet cake. The wet cake was reslurried in a mixture of water (1619 kg) and H₂SO₄ (85.04 kg) at 80-85° C. for five hours and further stirred at 25-30° C. for 16 hours. The solids were isolated by filtration, washed with water (1020 L) and dried under vacuum ay 50±5° C. for 36 hours to afford 446 kg of Compound 8-½H₂SO₄ as a yellow solid hemisulfate salt.

### Preparation of Compound 22

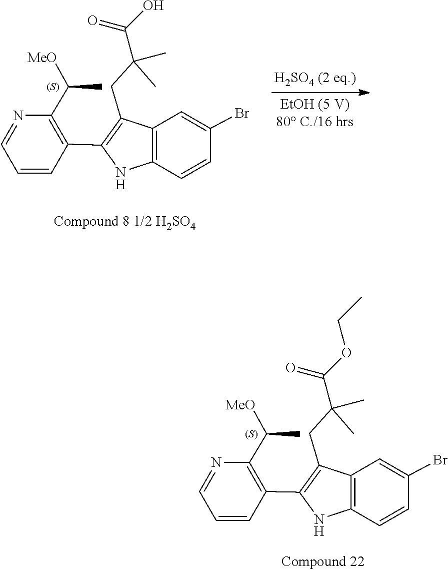

To a reactor R1 was charged Compound 8½H₂SO₄ (2.0 kg, 1.0 eq.) and EtOH (7 V) at 20-25° C. under nitrogen protection. Into the reactor was charged DMF (1.0% w/w) to form a suspension. Into the suspension was charged SOCl₂ (1.0 eq.) dropwise at 0-10° C. over 1 hour with agitation to afford a clear, homogenous solution. The reaction mixture was warmed to internal temperature of 60-65° C. and stirred for 16 hours. A sample by IPC showed the reaction was completed (Compound 8<2.5%; sample diluted with Ethanol). The reaction mixture was cooled to 20-25° C. and the pH was adjusted to ˜7.0 with addition of saturated NaHCO₃ aq. solution (˜10 V). Once the neutralization was completed, the reaction mixture was further agitated for 1 hour at 20-25° C. The mixture was filtered and the filter cake was washed with water (2 V). The wet cake was then slurried in water (7 V) for 5 hours. The slurry was then filtered and the cake was washed with water (2 V). The cake was further dried under high vacuum at <50° C. to afford Compound 22 (1,756 g; purity 98.9%; yield 96%; KF 0.02% water).

Alternatively, the carboxylic acid may be activated as a mixed anhydride (e.g., through use of isobutylchloroformate) prior to reduction in the next step, or the carboxylic acid may be directly reduced to the primary alcohol in the next step.

### Preparation of Compound 11a and 11b

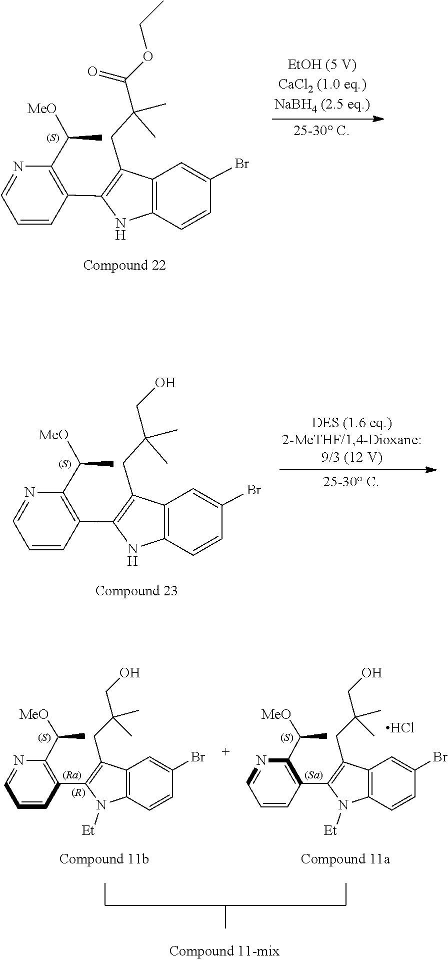

To a reactor was charged Compound 22 (100.0 g, 1.0 eq.), EtOH (500 mL, 5.0 vol.) and CaCl₂) (24.2 g, 1.0 eq.). The mixture was stirred at 25-30° C. for 30 mins. NaBH₄ (20.6 g, 2.5 eq.) was batch-added into the mixture at 25-30° C. The mixture was stirred at 25-30° C. for 16 h followed by sampling for IPC (HPLC purity: 97.2 A % of Compound 23 and 0.92 A % of Compound 22). The reaction mixture was adjusted to pH 1-2 with 3 M HCl (ca. 2.4 V) at 20-30° C. (inner temperature). The mixture was stirred at 20-30° C. for 30 mins. The reaction mixture was adjusted to pH 4.5-5.0 with 30% aq. NaOH (ca. 2.5 V) at 20-30° C. (inner temperature). The mixture was stirred at 20-30° C. for 30 mins. Water (500 mL, 5.0 vol.) was added into the mixture at 25-30° C. The mixture was stirred at 20-30° C. for 14 h. The mixture was filtered, and the filter cake was rinsed with water (200 mL, 2.0 vol.). The wet filter cake was dried at 55-60° C. under reduced pressure (−0.95 Mpa) for 14 h. 89 g of the Compound 23 was obtained as an off-white solid with 97.6% HPLC purity and 95% uncorrected yield.

To a reactor was charged 2-MeTHF (270 mL, 9.0 vol.), 1,4-dioxane (90 mL, 3.0 vol.), and Compound 23 (30.0 g, 1.0 eq.). KOH (7.3 g, 1.8 eq.) was batch-added into the mixture at 15-30° C. (inner temperature). Diethylsulfate (DES) (17.7 g, 1.6 eq.) was added dropwise into the mixture at 25-30° C.(IT). The mixture was stirred at 25-30° C. for 16 h followed by sampling for IPC [HPLC purity: 96.4 A % of Compound 11b (Ratio: 7.05 of Compound 11b/Compound 11a) and no detection of Compound 23. The mixture was stirred at 20-30° C. for 30 mins. Water (90 mL, 3.0 vol.) was added into the mixture at 25-30° C. and stirred for 30 mins, separated the organic phase. 2-MeTHF (60 mL, 2.0 vol.) was added into the aqueous phase at 20-30° C. and stirred for 30 mins, separated the aqueous phase. The combined organic phases were concentrated and swapped into THF (300 mL, 10.0 vol.). 6 M HCl (10.8 mL, 0.9 eq.) was dropwise added into the mixture at 20-30° C. The mixture was stirred at 20-30° C. for 13 h. The mixture was filtered, and the filter cake was rinsed with THF (60 mL, 2.0 vol.). The wet filter cake was dried at 50-55° C. under reduced pressure (−0.95 Mpa) for 14 h. 26.0 g of the Compound 11b-HCl was obtained as an off-yellow solid with 97.9% HPLC purity and 74% uncorrected yield.

### Alternative Synthesis Using the Intermediacy of Compound 24

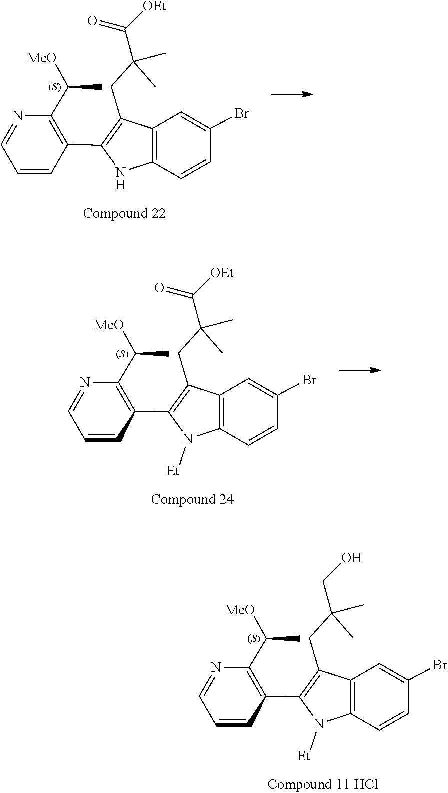

To a reactor was charged 2-MeTHF (150 mL, 5.0 vol.), 1.4-dioxane (150 mL, 5.0 vol.) and Compound 22 (30.0 g, 1.0 eq.). KOH (6.6 g, 1.8 eq.) was batch-added into the mixture at 20-30° C. The mixture was cooled to −5 to 0° C., and diethylsulfate (DES) (16.1 g, 1.6 eq.) was dropwise-added into the mixture at −5 to 0° C. The mixture was stirred at −5 to 5° C. for 24 h followed by sampling for IPC [HPLC purity: 99.0 A % of Compound 24 and 0.7 A % of Compound 22 (ratio of 5.3:1 of desired/undesired atropisomer)]. HOAc (7.1 g, 1.8 eq.) was dropwise-added into the reactor and stirred for 0.5 h, then the reaction mixture was warmed to 20-30° C. and 10% aq. NaCl (90 mL, 3.0 vol.) was added into the mixture at 20-30° C. The mixture was stirred for 30 mins. The aqueous phase was separated. The organic phase was washed with 10 wt % NaCl aq. solution (90 mL, 3.0 vol.), concentrated under reduced pressure at 30-40° C., and swapped to EtOH (150 mL, 5.0 vol.) to afford Compound 24 as a solution with 97.0% HPLC purity and 97% assay-corrected yield.

To a reactor was charged Compound 24 EtOH solution (142 g, equivalent to 30.9 g of neat Compound 24, 1.0 eq.) at 20-30° C. CaCl₂ (7.1 g, 1.0 eq.) was added into the mixture at 25-30° C. The mixture was stirred at 25-30° C. for 1 h. NaBH₄ (6.0 g, 2.5 eq.) was batch-added at 25-30° C. The mixture was stirred at 25-30° C. for 24 h followed by sampling for IPC (HPLC purity: 98.2 A % of Compound 11 and 0.3 A % of Compound 24). 3M HCl aq. solution (90 mL, 3.0 vol.) was dropwise-added to adjust pH to 1-2 at 25-30° C. and stirred for 0.5 h. H₂O (150 mL, 5.0 Vol.) was added and stirred for 0.5 h. 30 wt % NaOH aq. solution (14.5 mL, 0.46 Vol.) was dropwise-added to adjust pH to 4.5-5.0 at 25-30° C. and stirred for 0.5 h. AcONa (3.1 g, 0.6 eq.) was batch-added at 25-30° C. The mixture was warmed to 60-65° C. and stirred for 2 h. The mixture was cooled to 25-30° C. and stirred for 2 h. The mixture was filtered, and the finer cake was rinsed with H₂O (45 mL, 1.5 vol.). The wet finer cake was dried at 50-55° C. under reduced pressure for 14 h. 26.2 g of the Compound 11a and Compound 11b mixture was obtained as off-white solid with 99.3% HPLC purity (include undesired atropisomer: 16.6 A %) and 93% uncorrected yield. To a reactor was charged the Compound 11a and Compound 11b mixture (26.2 g) and THF (260 mL, 10 vol.) was added into a reactor. 6M HCl (9.5 mL, 0.9 eq.) was dropwise added into the mixture at 20-30° C. The mixture was stirred at 20-30° C. for 13 h. The mixture was filtered, and the finer cake was rinsed with THE (60 mL, 2.0 vol.). The wet filter cake was dried at 50-55° C. under reduced pressure for 14 h. 21.3 g of the Compound 11b was obtained as an off-yellow solid with 99.5% HPLC purity (exclude undesired product) and 75% uncorrected yield.

### Example 7. Separation of Compounds 11a and 11b in Flow

A mixture of Compound 11a and Compound 11b (freebase) and THF (10 V) were charged to a reactor. The mixture was stirred for two hours at 25±5° C. and then was filtered through diatomaceous earth (2.5 wt %). The filtrate was pumped through a flow reactor at 220° C. for a residence time of 240 seconds and then was rapidly cooled to 0° C. for a residence time of 120 seconds. The outflow, as a >1.58:1 mixture of atropisomer, was collected in a reactor. Compound 11b HCl seeds (0.05 wt %) were charged to the reactor. HCl in IPA (0.25 eq., ˜0.15 V) was charged to the reactor over no less than five hours while maintaining the temperature at 25±5° C. The mixture was further stirred at 25±5° C. for four hours. Additional HCl in IPA (0.25 eq., ˜0.15 V) was charged to the reactor over no less than five hours while maintaining the temperature at 25±5° C. The mixture was further stirred at 25±5° C. for four hours. The solids were isolated by centrifugation and washed with THF (0.5 V) to afford Compound 11b HCl. The isolated solids are slurried in IPA (8 V) at 60-70° C. for no less than five hours. The slurry is cooled to 10-20° C. over no less than five hours, further stirred for two hours and filtered. The isolated solids are washed with IPA (2×2.5 V) and dried under vacuum at 40±5° C.

The filtrate from centrifuge was passed through a chloride ion-exchange column to obtain the Compound 11a/11 b mix (freebase) which could be isomerized in the same manner as described above.

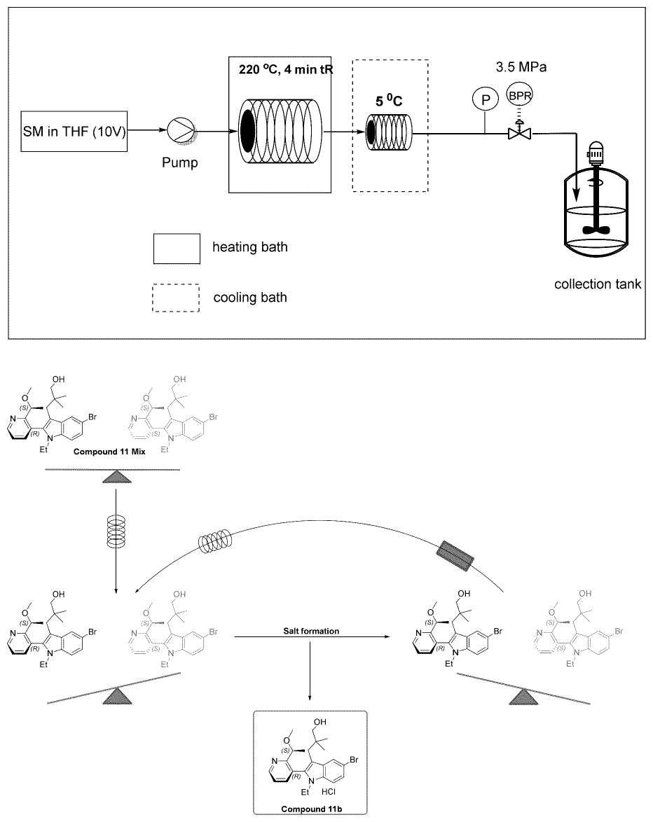

A schematic diagram of the flow process used to separate Compound 11b from Compound 11b is shown in FIG. 1.

### Example 8. Synthetic Procedure for Compound 3a

### Part 1—Synthesis or Compound 3h—tert-butyl (R)-2-formylmorpholine-4-carboxylate

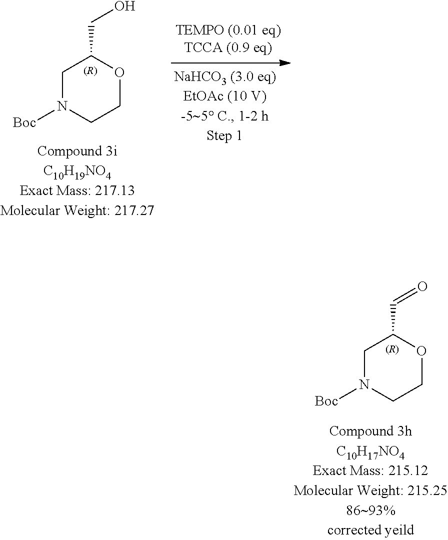

To a 3000 L glass-lined reactor (A) was charged tert-butyl (R)-2-(hydroxymethyl)morpholine-4-carboxylate (Compound 3i) (104 kg, 478.7 mol, 1.0 eq) and EtOAc (1032 kg, 11 v), followed NaHCO₃ (120.6 kg, 1436.1 mol, 3.0 eq) and TEMPO (0.75 kg, 4.787 mol, 0.01 eq) solution in EtOAc (94 kg, 1 v). The resulting mixture was purged with N₂ three times, and cooled to −15 to −10° C.

To a 2000 L reactor (B) was charged TCCA (100.1 kg, 430.8 mol, 0.9 eq) and EtOAc (938 kg, 10 V). The mixture was agitated until a clear solution was obtained. The resulting solution was purged with N₂ three times and cooled to −10 to −5° C. The mixture was then charged to the 3000 L glass lined reactor (A) while maintaining the temperature at −10 to 0° C. The resulting mixture was agitated at −5 to 0° C. for 1 h at which point GC monitoring showed the reaction was completion.

The reaction mixture was quenched by the addition of an aqueous solution of Na₂S₂O₃ (prepared by dissolving 520 kg of Na₂S₂O₃ into 832 kg of water) at 0-15° C. and agitated for 30 min at </=15° C. The phases were separated, and the rich organic phase was washed with water. The combined aqueous phases were extracted with EtOAc (703 kg×4, 7.5 v×4). The combined organics were then washed with 20 wt % brine (520 kg×2, 5 v×2). The organic phase was then concentrated at 15-35° C. under reduced pressure (−0.85 to −0.9 MPa) to 4-5 v to give an EtOAc solution of Compound 3h.

¹H NMR (400 MHz, CDCl₃, 25° C.) δ9.69 (s, 1H), 3.98-3.65 (m, 5H), 3.16-3.03 (m, 2H), 1.52 (s, 9H).

TABLE 7
GC method for Example 6, Part 1

| | |
|---|---|
| Instrument: | GC (e.g., Agilent 7860 series) |
| Column: | Agilent HP-5 (0.25 μm, 30.0 m × 0.32 mm) |
| Injection Temperature: | 270° C. |
| Split Ratio: | 30:1 |
| Control Mode: | Constant Flow |
| Flow Rate | 2.0 mL/min |
| Oven Temperature Program: | Initial 40° C., hold 3 min, 30° C. to 280° C., hold 4 min |
| Rum Time: | 15 min |
| FID Detector: | H₂: 40 mL/min |
| | Air: 400 mL/min |
| | N₂: 30 mL/min |
| | Temperature: 280° C. |
| Blank/Diluents: | MeCN |
| Sample Preparation: | Resolution Solution (0.6 mg/mL of Compound 3i and 20.0 |
| | mg/mL of Compound 3h): Weigh about 200.0 mg of |
| | working standard of Compound 3i into a 10 mL of |
| | volumetric flask, dissolve and fill to the designated volume |
| | with diluents to obtain a stock solution A. Weigh 200.0 mg of |
| | Compound 3h into a 10 mL volumetric flask, and transfer |
| | 0.3 mL of stock solution A, and dissolve and fill to the |
| | designated volume with diluents. |
| | Sample Solution (20.0 mg/mL): Transfer 1.0 mL reaction |
| | liquid into a 4 mL of centrifuge tube, add 1.5 mL of MeCN to |
| | dissolve. |
| SST | Blank should have no significant interference on the analysis |
| | of the sample solution. The resolution between each target |
| | peak should not be less than 1.5. |
| Injection Sequence | Blank: Inject at least twice |
| | Resolution Solution: Inject once |
| | Sample Solution: Inject once |
| Injection Volume: | 2 μL |
| Retention Times: | Compound 3i ≈ 8.8 min |
| | Compound 3h ≈ 8.4 min |

### Part 2—Synthesis of Compound 3g—tert-butyl (S,Z)-2-(2-(((benzyloxy)carbonyl)amino)-3-methoxy-3-oxoprop-1-en-1 yl)morpholine-4-carboxylate

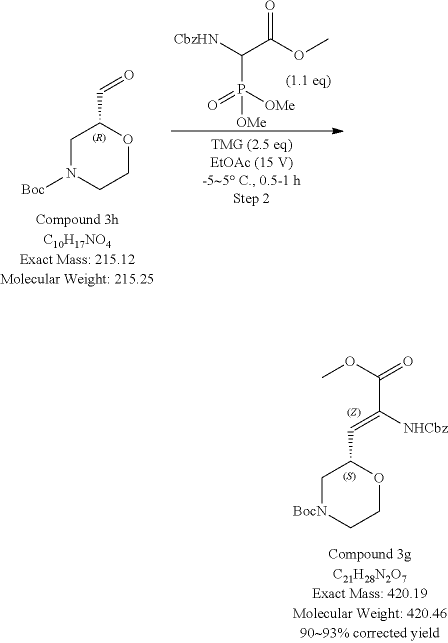

To a 3000 L reactor was charged the EtOAc solution of Compound 3h (763 kg, containing 675 kg of EtOAc and 82.4 kg 51, 382.8 mol, 1.0 eq), methyl 2-(((benzyloxy)carbonyl)amino)-2-(dimethoxyphosphoryl)acetate (139.4 kg, 421.1 mol, 1.1 eq) and EtOAc (437 kg, 15 v). The mixture was purged with N₂ three times and cooled to −5 to 0° C. Then tetramethylguanidine (110.2 kg, 957 mol, 2.5 eq) was charged dropwise at −5 to 5° C. and the reaction mixture was agitated for 0.5 h. Reaction monitoring by GC showed the reaction was complete.

The reaction mixture was charged into a 5000 L reactor containing H₂O (824 kg, 10 v) and cooled to −5-5° C. The resulting mixture was agitated for 0.5 h. The organic phase was separated, and the aqueous phase was extracted with EtOAc (400 kg×2, 5 v×2). The combined organic phases were washed successively with 5 wt % citric acid in 20 w % brine (400 kg×2, 5 v×2), 5 wt % aq. NaHCO₃ (400 kg×3, 5 v×3) and 20 wt % brine (400 kg×1,5 v×1), respectively. The resulting organic layer was concentrated under reduced pressure at 40-45° C. to about 1.5-2 v. THF (371 kg, 5 v) was charged to the residue and further concentrated to 1.5-2 v under reduced pressure at 40-45° C. The previous unit operation was repeated one more time to give Compound 3g as a THF solution in 91% HPLC assay-corrected yield and 100% ee by chiral HPLC.

¹H NMR (400 MHz, CDCl₃, 25° C.) δ7.33-7.27 (m, 5H), 7.03 (s, 1H), 6.05 (d, J=4.0 Hz, 1H), 5.10(dd, J=20, 12 Hz, 2H), 4.20-3.80 (m, 7H), 3.50-3.40 (m, 1H), 2.97-2.80 (m, 2H), 1.42 (s, 9H).

TABLE 8
GC method for reactor monitoring of Example 6, Part 2

| | |
|---|---|
| Instrument: | GC (e.g., Agilent 7860 series) |
| Column: | Agilent HP-5 (0.25 μm, 30.0 m × 0.32 mm) |
| Injection Temperature: | 270° C. |
| Split Ratio: | 30:1 |
| Control Mode: | Constant Flow |
| Flow Rate | 2.0 mL/min |
| Oven Temperature Program: | Initial 40° C., hold 3 min, 30° C. to 280° C., hold 4 min |
| Rum Time: | 15 min |
| FID Detector: | H₂: 40 mL/min |
| | Air: 400 mL/min |
| | N₂: 30 mL/min |
| | Temperature: 280° C. |
| Blank/Diluents: | MeCN |
| Sample Preparation: | Reference Solution of Compound 3h (5.0 mg/mL of |
| | Compound 3h): Weigh about 50.0 mg of working standard |
| | of Compound 3h into a 10 mL volumetric flask, dissolve, |
| | and fill to the designated volume with diluents. |
| | Sensitivity Solution of Compound 3h (0.05 mg/mL of |
| | Compound 3h): Transfer 1 mL of reference solution of |
| | Compound 3h into a 100 mL of volumetric flask, dissolve, |
| | and fill to the designated volume with diluents. |
| | Sample Solution: Transfer 2.0 mL reaction liquid into a 10 |
| | mL of volumetric flask, dissolve, and fill to the designated |
| | volume with diluents. |
| SST | The S/N of target peak of sensitivity solution of Compound |
| | 3h should not be less than 10.0. |
| Injection Sequence | Blank: Inject at least twice |
| | Reference Solution: Inject once |
| | Sensitivity Solution: Inject once |
| | Sample Solution: Inject once |
| Injection Volume: | 2 μL |
| Retention Times: | Compound 3h ≈ 8.4 min |

TABLE 9
HPLC method for Example 6, Part 2

| Instrument: | HPLC (e.g., Agilent 1260 series) | |
|---|---|---|
| Column: | Agilent PoroShell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.00 | 5 |
| | 10.0 | 90 |
| | 12.00 | 90 |
| | Post time: 3 min | |
| Flow Rate: | 1.0 | mL/min |
| UV Detector Wavelength: | 210 | nm |
| Column Temperature: | 30° C. | |
| Blank/Diluents | 1:1 (v/v) MeCN/water | |
| Sample Preparation: | Reference Solution of Compound 3g (1.0 mg/mL of | |
| | Compound 3g): Weigh about 25.0 mg of working standard | |
| | of Compound 3g into a 25 mL volumetric flask, dissolve, | |
| | and fill to the designated volume with diluents. | |
| | Sample Solution: Transfer 1.0 mL reaction liquid into a 50 | |
| | mL of volumetric flask, dissolve, and fill to the designated | |
| | volume with diluents. | |
| Injection Volume: | 2 | μL |

| | |
|---|---|
| Retention Times: | Compound 3g ≈ 7.4 min |

TABLE 10
Chiral HPLC method for Example 6, Part 2

| | |
|---|---|
| Instrument: | HPLC (e.g., Agilent 1260 series) |
| Column: | Daicel Chiralpak OZ-H (4.6 × 250 mm, 5.0 μm) |
| Mobile Phase: | A: 0.1% TFA in EtOH |
| | B: n-Hexane |
| | A:B = 40:60 |
| Flow Rate: | 0.8 mL/min |
| UV Detector Wavelength: | 210 nm |
| Column Temperature: | 30° C. |
| Run Time: | 15.0 min |
| Blank/Diluent: | EtOH |
| Sample Preparation: | Resolution Solution: Weigh about 10.0 mg of reference |
| | standard of (R)-Compound 3g into a 10 mL of volumetric |
| | flask, dissolve and fill to the designated volume with diluents |
| | to obtain a stock solution of (R)-Compound 3g. Weigh about |
| | 10.0 mg of reference standard of (S)-Compound 3g into a |
| | 10 mL of volumetric flask, and transfer 0.5 mL of stock |
| | solution of (R)-Compound 3g, dissolve and fill to the |
| | designated volume with diluents. |
| | Sensitivity Solution: Transfer 0.3 mL of stock solution of |
| | (S)-Compound 3g into a 100 mL of volumetric flask, |
| | dissolve, and fill to the designated volume with diluents. |
| | Sample Solution: Weigh about 10.0 mg sample into a 10 mL |
| | volumetric flask, dissolve, and fill to the designated volume |
| | with diluents. Prepare two replicates. |
| Injection Volume: | 2 μL |
| SST | 1. Blank should have no significant interference on the |
| | analysis of the sample solution. The resolution between each |
| | target peak ((R)-Compound 3g and (S)-Compound 3g) |
| | should not be less than 1.5. |
| | 2. The S/N of (S)-Compound 3g in sensitivity solution should |
| | not less than 10.0. |
| Injection Sequence | Blank: Inject at least twice |
| | Resolution Solution: Inject once |
| | Sensitivity Solution: Inject once |
| | Test sample 1: Inject once |
| | Test sample 2: Inject once |
| Retention Times: | (S)-Compound 3g ≈ 5.8 min |
| | (R)-Compound 3g ≈ 6.6 min |

### Part 3—Synthesis of Compound 3b salt with Compound 3f—(S,Z)-2-(((benzyloxy)carbonyl)amino)-3-(4-(tert-butoxycarbonyl)morpholin-2-yl)acrylic acid, (R)-1-phenylethan-1-amine salt

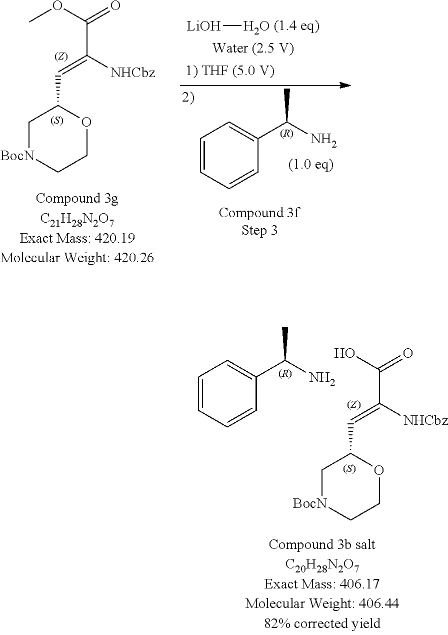

To a 2000 L reactor was charged a THF-solution of Compound 3g (204.5 kg comprising 106.3 kg of 52, 252.9 mol, 1.0 eq. and 109 L of THF) and THF (380 kg to 532 L, 5 v). The mixture was agitated at ambient temperature to afford a clear solution and then was cooled to 0-10° C. Aqueous UGH monohydrate (15 kg, 354.1 mol, 1.4 eq.) in water (266 L, 2.5 v) was charged to the cold solution at ≤10° C. over 3.5 h. After the addition was complete, the mixture was warmed to 10-15° C. and agitated at 10-15° C. for an additional 1 h. Reaction monitoring by HPLC showed the reaction is complete.

To the crude reaction mixture was charged 0.1 M eq. HCl (106 L, 1 v). The reaction mixture was concentrated under reduced pressure at 35-40° C. to 400 L. Water (532 L, 5 v) was charged into the mixture, and the mixture was extracted twice with MTBE (390 kg×2, 5 v×2). The aqueous phase was then cooled to 0-10° C. and 1 M eq. HCl (307 kg) was charged dropwise to a pH of 2-3. The resulting aqueous phase was further extracted twice times with MTBE (550 kg×1, 7 v×1, 390 kg×1, 5 v×1). The combined organic phases were washed with brine (500 kg×2, 5 v×2), and warmed to 20-30° C. and treated with Na₂SO₄ (100 kg, 1 w) and activated carbon (21 kg, 20 wt %). The mixture was agitated at 20-30° C. for 1 h and filtered. The spent solids were rinsed twice with MTBE (150 kg×2, 1.5 v×2). Compound 3f ((R)-(+)-1-phenylethylamine) (30.6 kg, 252.8 mol, 1.0 eq.) was charged dropwise to the filtrate solution at 10-25° C. over 30 min. The mixture was agitated at 10-25° C. for 18 h. The resulting solids were isolated by filtration and were washed twice with MTBE (150 kg×2, 1.5 v×2). The wet cake was dried at 40-45° C. under reduced pressure to afford 108 kg of Compound 3b salt as a white solid in 77.3% assay-corrected yield and 100% ee.

¹H NMR (400 MHz, CDCl₃, 25° C.) δ8.01 (brs, 2H), 7.37-7.27 (m, 10H), 5.77 (d, J=8.0 Hz, 1H), 5.08(dd, J=20, 12 Hz, 2H), 4.24-4.20 (m, 2H), 4.09-3.80 (m, 3H), 3.53-3.26 (m, 1H), 2.97-2.80 (m, 1H), 2.70-2.60 (m, 1H), 1.52-1.47 (m, 12H).

TABLE 11
HPLC method for reaction monitoring of Example 6, Part 3

| Instrument: | HPLC (e.g., Agilent 1260 series) | |
|---|---|---|
| Column: | Agilent PoroShell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.00 | 5 |
| | 7.00 | 90 |
| | 12.0 | 90 |

| | Post time: 3 min | |
|---|---|---|
| Flow Rate: | 1.0 | mL/min |
| UV Detector Wavelength: | 210 | nm |

| | |
|---|---|
| Column Temperature: | 30° C. |
| Blank/Diluents | 1:1 (v/v) MeCN/water |
| Sample Preparation: | Resolution Solution of Compound 3b (0.04 mg/mL of |
| | Compound 3g and 1.0 mg/mL of Compound 3b): Weigh |
| | about 25.0 mg of working standard of Compound 3g into |
| | a 25 of volumetric flask, dissolve and fill to the designated |
| | volume with diluents to obtain a stock solution of |
| | Compound 3g. Weigh about 25.0 mg of working standard |
| | of Compound 3b into a 25 of volumetric flask, and transfer |
| | 1 mL of stock solution of Compound 3g in it, dissolve and |
| | fill to the designated volume with diluents. |
| | Sample Solution: Transfer 0.4 mL reaction liquid into a 50 |
| | mL of volumetric flask, dissolve, and fill to the designated |
| | volume with diluents. |
| Injection Volume: | 2 □L |
| SST | Blank should have no significant interference on the |
| | analysis of the sample solution. The resolution between |
| | each target peak should not be less than 1.5. |
| Injection Sequence | Blank: Inject at least twice |
| | Resolution Solution: Inject once |
| | Sample Solution: Inject once |
| Retention Times: | Compound 3g ≈ 7.4 min |
| | Compound 3b ≈ 6.6 min |

TABLE 12
HPLC purity method for Compound 3b salt

| Instrument: | HPLC (e.g., Agilent 1260 series) | |
|---|---|---|
| Column: | Agilent PoroShell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.00 | 5 |
| | 7.00 | 90 |
| | 12.00 | 90 |
| | Post time: 3 min | |
| Flow Rate: | 1.0 | mL/min |
| UV Detector Wavelength: | 210 | nm |
| Column Temperature: | 30° C. | |
| Blank/Diluents | 1:1 (v/v) MeCN/water | |
| Sample Preparation: | Resolution Solution of Compound 3b salt (0.04 mg/mL | |
| | of Compound 3g and 1.0 mg/mL of Compound 3b salt): | |
| | Weigh about 25.0 mg of working standard of Compound | |
| | 3g into a 25 of volumetric flask, dissolve, and fill to the | |
| | designated volume with diluents to obtain a stock solution | |
| | of Compound 3g. Weigh about 25.0 mg of working | |
| | standard of Compound 3b salt into a 25 of volumetric | |
| | flask, and transfer 1 mL of stock solution of Compound 3g | |
| | in it, dissolve, and fill to the designated volume with | |
| | diluents. | |
| | Sample Solution: Weigh about 100.0 mg of Compound | |
| | 3b salt into a 100 of volumetric flask, dissolve, and fill to | |
| | the designated volume with diluents. Prepare two | |
| | replicates. | |
| Injection Volume: | 2 | μL |

| | |
|---|---|
| Injection Sequence | Blank: Inject at least twice |
| | Resolution Solution: Inject once |
| | Test sample preparation 1: Inject twice |
| | Test sample preparation 2: Inject twice |
| SST | Blank should have no significant interference on the |
| | analysis of the sample solution. The resolution between |
| | each target peak should not be less than 1.5. |
| Retention Times: | (R)-(+)-1-Phenylethylamine ≈ 3.1 min |
| | Compound 3g ≈ 7.4 min |
| | Compound 3b salt ≈ 6.6 min |

TABLE 13
Chiral HPLC method for Compound 3b salt

| | |
|---|---|
| Instrument: | HPLC (e.g., Agilent 1260 series) |
| Column: | Daicel Chiralpak OJ-3R (4.6 × 150 mm, 3.0 μm) |
| Mobile Phase: | A: 0.1% H₃PO₄ in Water |
| | B: MeCN |
| | A:B = 65:35 |
| Flow Rate: | 1.0 mL/min |
| UV Detector Wavelength: | 210 nm |
| Column Temperature: | 30° C. |
| Run Time: | 15.0 min |
| Blank/Diluent: | 50:50 (v/v) MeCN/Water |
| Sample Preparation: | Resolution Solution: Weigh about 10.0 mg of reference |
| | standard of (R)-Compound 3b into a 10 mL of volumetric |
| | flask, dissolve and fill to the designated volume with diluents |
| | to obtain a stock solution of (R)-Compound 3b. Weigh |
| | about 10.0 mg of reference standard of (S)-Compound 3b |
| | into a 10 mL of volumetric flask, and transfer 0.5 mL of stock |
| | solution of (R)-Compound 3b, dissolve and fill to the |
| | designated volume with diluents. |
| | Sensitivity Solution: Transfer 0.3 mL of stock solution of |
| | (R)-Compound 3b into a 100 mL of volumetric flask, |
| | dissolve, and fill to the designated volume with diluents. |
| | Sample Solution: Weigh about 10.0 mg sample into a 10 mL |
| | volumetric flask, dissolve, and fill to the designated volume |
| | with diluents. Prepare two replicates. |
| Injection Volume: | 2 μL |
| SST | 1. Blank should have no significant interference on the |
| | analysis of the sample solution. The resolution between each |
| | target peak ((R)-Compound 3b and (S)-Compound 3b) |
| | should not be less than 1.5. |
| | 2. The S/N of (R)-Compound 3b in sensitivity solution |
| | should not less than 10.0. |
| Injection Sequence | Blank: Inject at least twice |
| | Resolution Solution: Inject once |
| | Sensitivity Solution: Inject once |
| | Test sample 1: Inject once |
| | Test sample 2: Inject once |
| Retention Times: | (S)-Compound 3b salt ((S)-Compound 3b) ≈ 5.1 min |
| | (R)-Compound 3b salt ((R)-Compound 3b) ≈ 6.4 min |

### Part 4a—Synthesis of Compound 3c—(S)-2-(((benzyloxy)carbonyl)amino)-3-((S)-4-(tert-butoxycarbonyl)morpholin-2-yl)propanoic acid

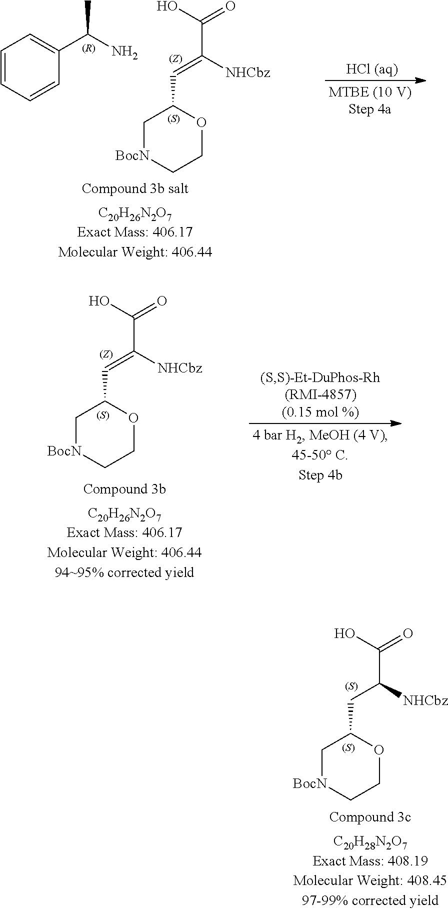

To a 3000 L reactor was charged Compound 3b salt (100 kg, 95.37 wt % qNMR), MTBE (740 kg, v) and water (500 kg, 5 v). The mixture was agitated for 10 min and then was cooled to 0-5° C. The pH of the mixture was adjusted to pH 2 using 1 M HCl(aq) (comprising 270 kg of water and 36 kg of 30% aq. HCl) at ≤10° C. The mixture was agitated for 10 min. The phases were separated. The isolated aqueous phase was further adjusted to pH 3.5 using 1 M HCl(aq) and extracted with MTBE (370 kg, 5 v). The combined organic layers were washed successively with aq. HCl (0.05 M, comprising 100 kg of water and 0.5 kg of 30% HCl, 1 v) and brine (200 kg, 2 v×3). The washed organic phase was warned to 15-25° C., and treated with Na₂SO₄ (100 kg, 1 w) and activated carbon (10 kg, 10 wt %). The mixture was agitated at 15-25° C. for 1 h and filtered through a pad of diatomite (20 kg, 0.2 w). The spent solids were rinsed twice with MTBE (150 kg×2, 1.5 v×2). The combined organic filtrates were further filtered through an organic membrane and the filtrate was concentrated to about 2 v (200 L) under reduced pressure at 35-40° C. MeOH (400 kg, 5 v) was charged to the resulting residue; the solution was once again concentrated to about 2 v (about 200 L) under reduced pressure at 35-40° C. The operation was repeated two more times to give Compound 3b as a MeOH solution (301.5 kg, 25.5 wt % by HPLC assay, comprising 77 kg of 52a).

The MeOH solution of Compound 3b was transferred to a 1000 L pressure reactor and diluted with MeOH (160 kg, 2.4 v, total 6 v). The mixture was purged with N₂ three times and further degassed by bubbling N₂ at a rate of 8.5 L/min for 2 h at 15-25° C. A solution of (S,S)-Et-DuPhos-Rh (207.5 g, 0.287 mol, 0.152 mol %) in degassed MeOH (1.8 L), prepared in a glove box, was charged into the pressure reactor under reduced pressure. The reactor was then purged with N₂ three times followed by H₂ three times. The reaction solution was agitated at 35-40° C. while maintaining the H₂ pressure at 4-10 atm for 5 h. Reaction monitoring by HPLC showed the reaction was complete. The pressure in the reactor was released and the reactor purged with N₂.

A saturated aqueous solution of NaHCO₃ (200 kg, 2.5 v, comprising 180 kg of water and 20 kg of NaHCO₃) was charged into the reaction mixture. The reaction mixture was concentrated to 3-4 v under reduced pressure at 45-50° C. and then diluted with water (200 kg, 2.5 v). The pH was adjusted with aqueous Na₂CO₃ solution (42 kg of water and 18 kg of Na₂CO₃). The mixture was agitated for 30 min. The phases were separated. The aqueous phase was washed with MTBE (185 kg×3, 2.5 v×3). The washed aqueous phase was partitioned again with MTBE (370 kg, 5 v) and the pH was adjusted to pH 2 with 1 M HCl(aq) (450 kg of water and 61.3 kg of 30% HCl) at ≤10° C. The phases were separated, and the pH of the aqueous phase was further adjusted to 2-4 with 1 M HCl(aq). The acidic aqueous phase was then extracted with MTBE (370 kg×2, 5 v×2). The combined organic layers were washed with brine (230 kg×2, 3 v×2) and treated with Na₂SO₄ (100 kg, 1 w) and activated carbon (3.9 kg, 5 wt %) for 1 h at 15-25° C. The mixture was agitated and then filtered through a pad of diatomite (15 kg, 0.2 w). The spent drying agent and carbon were washed with MTBE (150 kg×2, 2 v×2). The combined filtrates were concentrated to about 2 v (about 150 L) under reduced pressure at 35-45° C., followed by the addition of DCM (500 kg, 5 v) to the residue. The mixture was further concentrated to about 2 v (about 150 L) under reduced pressure at 35-45° C. The previous unit operation was repeated 2 times to give Compound 3c as a light-yellow DCM solution (221.2 kg, 32.1 wt % by HPLC assay, comprising 71 kg of 57) in a 96% corrected yield from Compound 3b salt.

¹H NMR (400 MHz, CDCl₃, 25° C.) δ8.68 (brs, 1H), 7.36-7.28 (m, 5H), 6.05 (app s, 1H), 5.13(dd, J=20, 12 Hz, 2H), 4.59 (app s, 1H), 3.86-3.45 (m, 5H), 3.00-2.80 (m, 1H), 2.60-2.50 (m, 1H), 2.00-1.90 (m, 2H), 1.47 (s, 9H).

TABLE 14
HPLC method for reaction monitoring of step 4b

| Instrument: | HPLC (e.g., Agilent 1260 series) | |
|---|---|---|
| Column: | OZ-RH (4.6 × 150 mm, 5.0 μm) | |
| Mobile Phase: | A: 0.1% H₃PO₄ in water; | |
| | B: MeCN | |
| | Time (min) | B (%) |
| Gradient: | 0.00 | 45 |
| | 10.0 | 60 |
| | 14.0 | 95 |
| | 20.0 | 95 |
| | Post time: 5 min | |
| Flow Rate | 0.6 | mL/min |
| UV Detector Wavelength: | 210 | nm |
| Column Temperature: | 30° C. | |
| Blank/Dilute: | 50:50 (v/v) MeCN/Water | |
| Sample Preparation: | Resolution Solution (0.2 mg/mL Compound 3b and 1.0 | |
| | mg/ mL Compound 3c): Weigh 10.0 mg of Compound 3b | |
| | into a 10 mL of volumetric flask, dissolve, and fill to the | |
| | designated volume with diluents to obtain a stock solution | |
| | of Compound 3b. Weigh 25 mg of Compound 3c into a | |
| | 25 mL volumetric flask, and transfer 0.5 mL of stock | |
| | solution of Compound 3b, and dissolve and fill to the | |
| | designated volume with diluents. | |
| | Sample Solution (1.0 mg/mL): Transfer 0.4 mL reaction | |
| | liquid into a 50 mL of volumetric flask, dissolve, and fill to | |
| | the designated volume with diluents. | |
| SST | Blank should have no significant interference on the | |
| | analysis of the sample solution. The resolution between | |
| | each target peak should not be less than 1.5. | |
| Injection Sequence | Blank: Inject at least twice | |
| | Resolution Solution: Inject once | |
| | Sample Solution: Inject once | |
| Injection Volume: | 2 | μL |

| | |
|---|---|
| Retention Times: | Compound 3b salt ≈ 7.0 min |
| | (R,S)-Compound 3c ≈ 7.5 min |
| | (S,S)-Compound 3c ≈ 8.2 min |

TABLE 15
HPLC purity method for Compound 3c

| Instrument: | HPLC (e.g., Agilent 1260 series) | |
|---|---|---|
| Column: | OZ-RH (4.6 × 150 mm, 5.0 μm) | |
| Mobile Phase: | A: 0.1% H₃PO₄ in water; | |
| | B: MeCN | |
| | Time (min) | B (%) |
| Gradient: | 0.00 | 45 |
| | 10.0 | 60 |
| | 14.0 | 95 |
| | 20.0 | 95 |
| | Post time: 5 min | |

| | |
|---|---|
| Flow Rate | 0.6 mL/min |
| UV Detector Wavelength: | 210 nm |
| Column Temperature: | 30° C. |
| Blank/Dilute: | MeCN |
| Sample Preparation: | Resolution Solution (0.2 mg/mL Compound 3b and 1.0 mg/mL |
| | Compound 3c): Weigh 10.0 mg of Compound 3b into a 10 mL of |
| | volumetric flask, dissolve, and fill to the designated volume with |
| | diluents to obtain a stock solution of Compound 3b. Weigh 25 mg of |
| | Compound 3c into a 25 mL volumetric flask, and transfer 0.5 mL of |
| | stock solution of Compound 3b, and dissolve and fill to the designated |
| | volume with diluents. |
| | Sensitivity Solution (0.01 mg/mL): Weigh 10.0 mg of Compound 3c |
| | into a 10 mL of volumetric flask, dissolve, and fill to the designated |
| | volume with diluents to obtain a solution containing Compound 3c at |
| | 1.0 mg/mL. And transfer 100 μL of the solution into a 100 mL of |
| | volumetric flask, dissolve, and fill to the designated volume with |
| | diluents to obtain a sensitivity solution. |
| | Reference Solution (1.0 mg/mL): Weigh accurately 25 mg reference |
| | standard of Compound 3c into a 25 mL volumetric flask, dissolve, and |
| | fill to the designated volume with diluents. Prepare two replicates. |
| | Sample Solution (1.0 mg/mL): Weigh accurately 250 mg test sample |
| | into a 50 mL volumetric flask, dissolve, and fill to the designated |
| | volume with diluents. Prepare two replicates. |
| Injection Sequence | Blank: Inject at least twice |
| | Sensitivity solution: Inject once |
| | Resolution solution: Inject 1 times |
| | Reference solution 1: Inject twice |
| | Reference solution 2: Inject once |
| | Test sample 1: Inject twice |
| | Test sample 2: Inject twice |
| SST | 1. Blank has no interference on the analysis of sample solution. |
| | 2. The resolution between two closer peaks should not be less than |
| | 1.5. |
| | 3. The S/N of the main peak in sensitivity solution chromatogram |
| | should be not less than 10. |
| | 4. Calculate the recovery of reference solution 2 with respect to |
| | reference solution 1 (use the mean area of two injections). The |
| | recovery should be in 98.0-102.0%. |
| Injection Volume: | 2 μL |
| Data Analysis & Reporting | 4. For each sample solution injection, integrate Compound 3c and |
| | all impurities except the peaks existing in the blank to determine the peak |
| | area and % peak area for each peak, and report all impurity peaks ≥ |
| | 0.10%. |
| | 5. For each sample injection, calculate the purity of Compound 3c |
| | and individual impurities in the sample using the following equation: |
| | $\% Component = \frac{{A}_{component}}{{A}_{total}} \times 100$ |
| | Where: |
| | A_{component} = Peak area of an individual component |
| | Aₜₒₜₐₗ = Sum of peak areas of all integrated peaks ≥ 0.10% |
| | 6. Report the average % area of Compound 3c for each sample |
| | from the duplicate preparations. |
| | This is the chromatographic purity of the sample. |
| | 7. Report to one decimal place of the purity of Compound 3c and to |
| | two decimal places of impurities. |
| | 8. For each sample injection, calculate the assay of Compound 3c in |
| | the sample using the following equation: |
| | $Assay ( w \% ) = \frac{{A}_{sample \times {C}_{std} \times {P}_{std}}}{{A}_{std} \times {C}_{sample}} \times 100$ |
| | Where: |
| | A_{std}: the mean peak area of Compound 3c in the reference solution 1 |
| | chromatogram (2 reference solution 1). |
| | C_{std}: the concentration of the reference standard (mg/mL). |
| | p_{std}: the potency of the reference standard. |
| | Aₛₐₘₚₗₑ: the peak area of Compound 3c in the sample chromatogram. |
| | Cₛₐₘₚₗₑ: the concentration of the sample (mg/mL). |
| Retention Times: | Compound 3b ≈ 7.0 min |
| | (R,S)- Compound 3c ≈ 7.5 min |
| | (S,S)- Compound 3c ≈ 8.2 min |

TABLE 1
Chiral HPLC method for Compound 3c

| Instrument: | HPLC (e.g., Agilent 1260 series) | |
|---|---|---|
| Column: | Chiralcel AD-H (4.6 × 250 mm, 5.0 μm) | |
| Mobile Phase: | A: 0.1% TFA in EtOH | |
| | B: n-Hexane | |
| | Time (min) | % A |
| Gradient: | 00.00 | 25 |
| | 15.00 | 25 |
| Flow Rate: | 1.0 | mL/min |
| UV Detector Wavelength: | 210 | nm |
| Column Temperature: | 30° C. | |
| Run Time: | 15.0 | min |
| Blank/Diluent: | i-PrOH | |
| Sample Preparation: | Resolution Solution (0.04 mg/mL of (R,R)-Compound 3c and | |
| | 0.04 mg/mL of (R,S)-Compound 3c and 0.04 mg/mL of (S,R)- | |
| | Compound 3c and 2.0 mg/mL of (S,S)-Compound 3c): | |
| | Respectively weigh about 20.0 mg (The mark of the solution state | |
| | needs to be substituted into the mass percentage calculation) of | |
| | mark of (R,R)-Compound 3c, (R,S)-Compound 3c and (S,R)- | |
| | Compound 3c into a 10 mL of volumetric flask, dissolve and fill | |
| | to the designated volume with diluents to obtain a stock solution | |
| | A. Weigh 20 mg of (S,S)-Compound 3c into a 10 mL volumetric | |
| | flask, and transfer 200 μL of stock solution of A, and dissolve and | |
| | fill to the designated volume with diluents. | |
| | Sensitivity Solution (0.003 mg/mL of (R,R)-Compound 3c): | |
| | Weigh about 15.0 mg (The mark of the solution state needs to be | |
| | substituted into the mass percentage calculation) of mark of | |
| | (R,R)-Compound 3c into a 10 mL of volumetric flask, dissolve | |
| | and fill to the designated volume with diluents to obtain a stock | |
| | solution B, and transfer 200 μL of stock solution of B into a 100 | |
| | mL volumetric flask, and dissolve and fill to the designated | |
| | volume with diluents. | |
| | Sample Solution (2.0 mg/mL): Weigh about 20.0 mg sample into | |
| | a 10 mL volumetric flask, dissolve, and fill to the designated | |
| | volume with diluents. Prepare two replicates. | |
| Injection Volume: | 5 | μL |

| | |
|---|---|
| SST | 1. Blank should have no significant interference on the analysis |
| | of the sample solution. The resolution between each target |
| | peak should not be less than 1.5. |
| | 2. The S/N of the main peak in sensitivity solution |
| | chromatogram should be not less than 10. |
| Injection Sequence | Blank: Inject at least twice |
| | Sensitivity solution: Inject once |
| | Resolution Solution: Inject once |
| | Test sample 1: Inject once |
| | Test sample 2: Inject once |
| Retention Times: | (S,S)-Compound 3c ≈ 7.1 min |
| | (S,R)-Compound 3c ≈ 8.2 min |
| | (R,S)-Compound 3c ≈ 8.8 min |
| | (R,R)-Compound 3c ≈ 10.4 min |

### Part 4b—Alternative Synthesis of Compound 3c

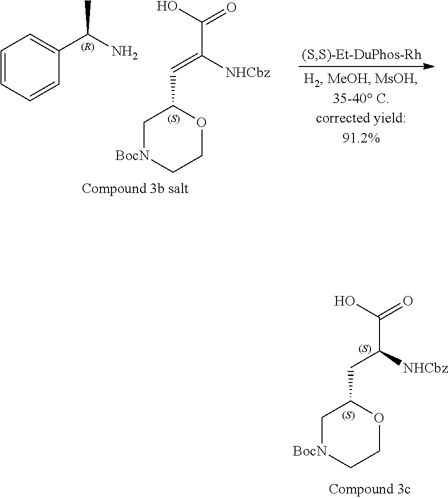

Alternatively, Compound 3b can be directly hydrogenated as the salt without initial formation of Compound 3b free base.

In this procedure, Compound 3b salt (150 kg) and MeOH (420 kg, 3.5 v) were charged into a reactor. The reactor and the contents were purged three time with N₂. The solution was cooled to between −5 and 5° C. and was treated slowly with MsOH (26.78 kg, 0.98 eq.) while maintaining the temperature at −5 to 5° C. The solution was warmed to 10 to 20° C. and transferred to a hydrogenation reactor. The reactor and contents was purged three time with N₂ before being treated with a solution of (S,S)-Et-DuPhos-Rh (308.2 g, 0.15 mol %) in MeOH (1.5 L). The reaction mixture was purged three times with N₂, followed by three times with hydrogen gas. The reaction was stirred at 35-40° C. under 10 atm H₂ for five hours. Reaction monitoring by HPLC showed the reaction was complete with ≤0.3% of Compound 3b detected. The reaction mixture was cooled to 15-25° C., purged with N₂, transferred to a reactor, diluted with 5 wt % eq. NaHCO₃ (600 kg), and concentrated to 5-6 V under reduced pressure (−0.0955 MPa to −0.0968 MPa) at 45-50° C. The concentrate was diluted with water (150 kg, 1 v), adjusted to pH 9-10 using 20 wt % eq. Na₂CO₃ (180 kg), and partitioned with MTBE. (222 kg, 2 v). The rich aqueous layer was washed twice with MTBE (2×222 kg), partitioned with MTBE (390 kg, 3.5 v), and adjusted to pH 1-3 using 1.5 M HCl (669 kg) at 0-10° C. The phases were separated, and the aqueous layer was further extracted twice with MTBE (2×333 kg). The combined organic layers were washed with 0.05 M eq. HCl (301.5 kg), twice with 20 wt % brine (2×300 kg). The organic layer was treated with Na₂SO₄ (150 kg, 1 wt %) and activated carbon (7.6 kg, 5 wt %), was stirred for 1 h at 15-25° C. and was filtered through diatomaceous earth (7.5 kg, 0.05 wt %). The filter aid was washed three times with MTBE (3×112 kg). The combine filtrates solvent swapped under reduced pressure at 35-45° C. using a total of 7 V of dichloromethane to afford 422.3 g (25 wt %) of a light yellow-colored solution of Compound 3c.

### Part 5—Synthesis of Compound 3e—tert-butyl (S)-2-((S)-2-(((benzyloxy)carbonyl)amino)-3-((S)-3-(methoxycarbonyl)tetrahydropyridazin-1(2H)-yl)-3-oxopropyl)morpholine-4-carboxylate

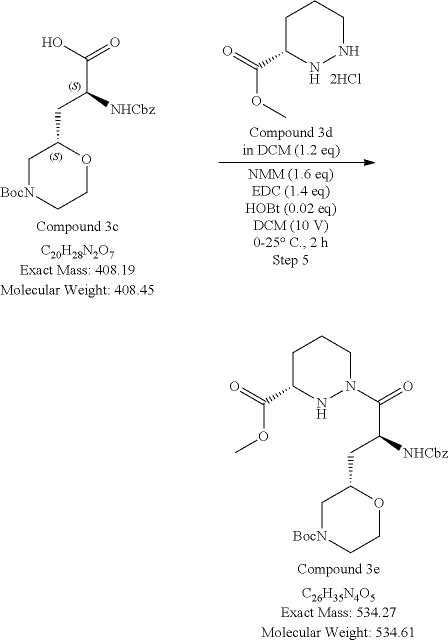

To a 2000 L glass-lined reactor was charged a DCM solution of Compound 3d (172 kg, 26.4 wt %, net weight: 45.4 kg, 1.2 eq) and DCM (940 kg, 10 v). The reactor was then purged with N₂ two times and cooled to 0-5° C. N-Methylmorpholine (31.6 kg, 1.8 eq) was charged to the mixture over a period of 10 min at 0-5° C., followed by DCM solution of Compound 3c (221 kg, 32.1 wt %, net weight: 70.9 kg, 1.0 eq) at 0-5° C. The mixture was agitated at 0-5° C. for 10 mins. HOBt (470 g, 0.02 eq.) and EDCl (46.6 kg, 1.4 eq.) were charged to the mixture at 0-5° C. The reaction mixture was agitated at 0-5° C. for 2 h. Reaction monitoring by HPLC showed the reaction is complete.

The crude reaction mixture was washed successively three times with water (710 kg×3, 10 v×3) and one time with brine (910 kg, 10 v). The organic solution was partially concentrated under reduced pressure to ca. 200 L prior to the addition of MeOH (168 kg). The mixture was further concentrated under reduced pressure to ca. 200 L. The previous unit operation was repeated one more time to afford Compound 3e as a MeOH solution (319 kg solution), which was used for the next step without further purification.

¹H NMR (400 MHz, CDCl₃, 25° C.) N/A

TABLE 27
HPLC method for reaction monitoring of Example 6, Part 5

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.0 | 5 |
| | 7.0 | 90 |
| | 12.0 | 90 |
| | Post time: 4.0 min | |
| Flow Rate | 1.0 | mL/min |
| UV Detector Wavelength: | 210 | nm |
| Column Temperature: | 30° C. | |
| Blank/Dilute: | MeCN | |
| Sample Preparation: | Sample Solution: Pipette 0.7 mL of reaction solution into a 50 mL | |
| | volumetric flask, add 1 mL of purified water and bring to the | |
| | volume with diluent. Mix well and filtrate the solution for injection. | |
| Injection Volume: | 2 | μL |

| | |
|---|---|
| Retention Times: | Compound 3c ≈ 6.5 min |
| | Compound 3e ≈ 7.0 min |
| | EDCI ≈ 1.2-1.3 min |
| | HOBt ≈ 3.3-3.4 min |
| | DCM ≈ 5.0 min |

TABLE 38
HPLC purity method for Compound 3e

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.0 | 5 |
| | 10.0 | 40 |
| | 15.0 | 40 |
| | 24.0 | 60 |
| | 27.0 | 90 |
| | 35.0 | 90 |
| | Post time: 5.0 min | |
| Flow Rate | 1.0 | mL/min |
| UV Detector Wavelength: | 210 | nm |
| Column Temperature: | 30° C. | |
| Blank/Dilute: | MeCN | |
| Sample Preparation: | Sample solution (0.5 mg/mL of Compound 3e): Accurately | |
| | weigh 150 mg of sample solution (ca. 30 wt %) into a 100 mL | |
| | volumetric flask containing one third of diluent, bring to the | |
| | volume with diluent, mix well. Prepare each sample in duplicate. | |
| Injection Volume: | 2 | μL |

| | |
|---|---|
| Retention Times: | Compound 3c ≈ 16.8 min |
| | Compound 3e ≈ 20.2 min |

### Part 6—Synthesis of Compound 3a—(S)-1-((S)-2-(((benzyloxy)carbonyl)amino)-3-((S)-4-(tert-butoxycarbonyl)morpholin-2 yl)propanoyl)hexahydropyridazine-3-carboxylic acid

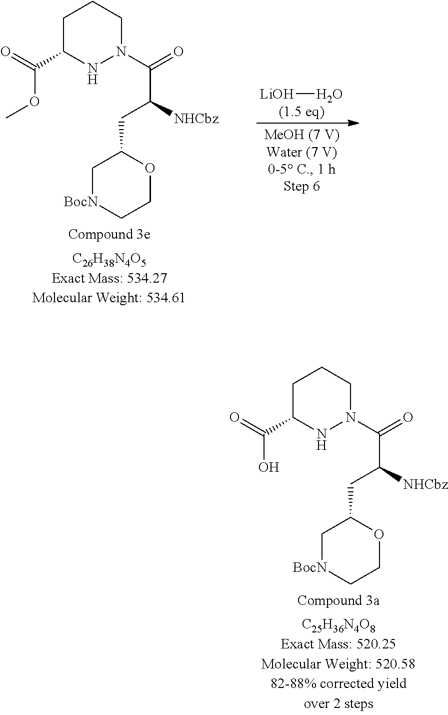

To a 2000 L glass-lined reactor was charged Compound 3e in MeOH (319 kg, 29.1 wt %, net weight: 92.9 kg, 1.0 eq) and MeOH (514 kg, 7 v). The reactor was purged with N₂ two times and cooled to 3-4° C. An aqueous UGH solution was prepared by dissolving 11 kg of LiOH·H₂O (1.5 eq.) in 650 kg of water (7 v) in a 1000 L glass-lined reactor. The aqueous base was charged over a period of 2 h at 0-5° C. to the initial mixture. The mixture was agitated at 0-5° C. for 2 h. HPLC monitoring showed the reaction was complete.

The reaction mixture was neutralized to pH 6-7 by using 1 M HCl aqueous at 0-15° C. and was concentrated under reduced pressure to remove most of the organic solvents. After concentration, MTBE (688 kg, 10 v) was charged to the residue followed by adjusting the pH of the mixture to pH 3-4 at or below 15° C. using aqueous 1 M HCl. After the pH adjustment, solid NaCl (28 kg, 0.3 wt) was charged, and the mixture was agitated for 1 h. The phases were separated. The organic phase washed with 20 wt % NaCl aqueous solution (930 kg, 10 v), treated with seed crystals of Compound 3a (1.4 kg, 1.5 wt %) and agitated at 15-25° C. for 20 h. n-Heptane (635 kg, 10 v) was then charged into the resulting slurry at 15-25° C. over a period of 5 h. The slurry was cooled to 0-5° C. and agitated for an additional 2 h. The solids were isolated by filtration, washed with 1:1 (v/ti) MTBE/n-heptane (130 kg×2) and dried at 45-55° C. under high vacuum (4-7 mbar) for 18 h to give Compound 3a as a white solid (84 kg, 98.9 wt % assay by qNMR in a 90.3% assay-corrected yield over two steps from Compound 3c.

¹H NMR (400 MHz, DMSO-d6, 25° C.) δ12.73 (brs, 1H), 7.38-7.31 (m, 5H), 7.12 (d, J=12 Hz, 1H), 5.07-4.95 (m, 4H), 4.05 (d, J=12 Hz, 1H), 3.74-3.65 (m, 3H), 3.35-3.20 (m, 3H, overlapped with residue water peak in DMSO-d6), 2.90-2.75 (m, 2H), 2.50 (app brs, 1H), 1.85-1.75 (m, 1H), 1.54-1.52 (m, 2H), 1.50-1.45 (m, 3H), 1.39 (s, 9H).

TABLE 4
HPLC method for reaction monitoring of Example 6, Part 6

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% H₃PO₄ in water | |
| | B: MeCN | |
| | Time (min) | % B |
| Gradient: | 0.0 | 5 |
| | 7.0 | 90 |
| | 12.0 | 90 |
| | Post time: 4.0 min | |
| Flow Rate | 1.0 | mL/min |
| UV Detector Wavelength: | 210 | nm |
| Column Temperature: | 30°C | |
| Blank/Dilute: | 1:1 (v/v) MeCN/water | |
| Sample Preparation: | Sample Solution: Pipette0.7 mL of reaction solution into a 50 mL | |
| | volumetric flask, dissolve and bring to the volume with diluent. | |
| | Mix well and filtrate the solution for injection. | |
| Injection Volume: | 2 | μL |

| | |
|---|---|
| Retention Times: | Compound 3e ≈ 7.0 min |
| | Compound 3a ≈ 6.3 min |

TABLE 20
HPLC purity method for Compound 3a

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% H3₃PO₄ in water | |
| | B: MeCN | |
| | Time (min) | % B |
| Gradient: | 0.0 | 5 |
| | 10.0 | 140 |
| | 15.0 | 40 |
| | 24.0 | 60 |
| | 27.0 | 90 |
| | 35.0 | 90 |
| | Post time: 5.0 min | |
| Flow Rate | 1.0 | mL/min |
| UV Detector Wavelength: | 210 | nm |
| Column Temperature: | 30° C. | |
| Blank/Dilute: | 1:1 (v/v) MeCN/water | |
| Sample Preparation: | Sample Solution (1.0 mg/mL of Compound 3a): Accurately weigh | |
| | 50 mg of sample into a 50 mL volumetric flask, dissolve and bring | |
| | to the volume with diluent, mix well. Prepare each sample in | |
| | duplicate. | |
| Injection Volume: | 5 | μL |

| | |
|---|---|
| Retention Times: | (S,R,S)- Compound 3a ≈ 14.3 min |
| | (R,S,S)- Compound 3a ≈ 14.8 min |
| | (S,S,S)- Compound 3a ≈ 15.3 min |
| | Compound 3c ≈ 17.3 min |
| | Compound 3e ≈ 20.7 min |

TABLE 21.5
Chiral HPLC method for Compound 3a

| | |
|---|---|
| Instrument: | HPLC (e.g., Agilent 1260 series) |
| Column: | DAICEL CHIRALCELOJ-3R (4.6 × 150 mm, 3.0 μm) |
| Mobile Phase: | A: 0.1% H₃PO₄ in water |
| | B: MeCN |
| Isocratic: | A:B = 73:27 |
| Flow Rate: | 0.3 mL/min |
| UV Detector Wavelength: | 210 nm |
| Column Temperature: | 35°C |
| Run Time: | 70.0 min |
| Diluent/Blank: | MeCN |
| Injection Volume: | 2 μL |
| Sample Preparation: | Sample Solution: Accurately weigh 25 mg of sample, transfer |
| | into a 25mL volumetric flask, add sufficient diluent to dissolve, |
| | bring to the volume with diluent and mix well. Prepare each |
| | sample in duplicate. |
| Retention Times: | (S,R,S)-Compound 3a ≈ 21.4 min |
| | (S,S,R)-Compound 3a ≈ 23.0 min |
| | (R,S,S)-Compound 3a ≈ 27.5 min |
| | (S,S,S)-Compound 3a ≈ 29.7 min |
| | (R,R,R)-Compound 3a ≈ 38.5 min |

### Example 9. Synthetic Procedure for Compound 2

Compound 17 can be prepared as described, for example, in WO 2021/091982.

### Part 1—Synthesis of Compound 2 (Barium Salt

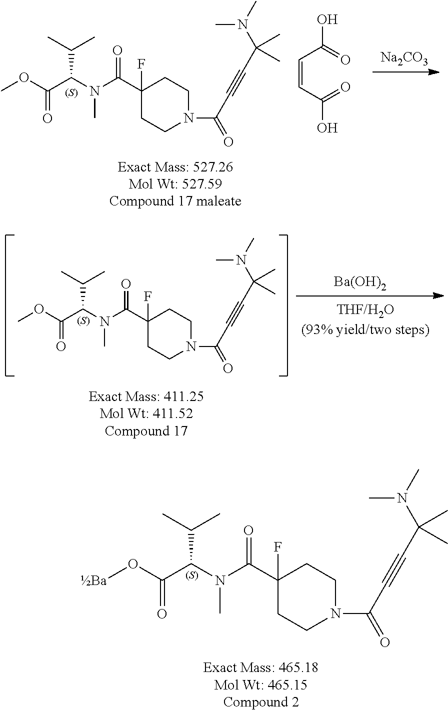

MTBE (270.35 kg, 8 V) and water (451.00 kg, 10 V) were charged to a reactor at 25±5° C. Compound 17 maleate (45.65 kg, 1.0 eq) and Na₂CO₃ (18.9 kg, 2.1 eq. (2.08-2.12 eq.)) were successively charged to the at 25±5° C. Additional MTBE (66.65 kg, 2 V) was charged to the reactor at 25±5° C. The mixture was stirred at 25±5° C. until all solids were dissolved. The phases were separated. The isolated lower lean aqueous layer was extracted once with MTBE (166.40 kg, 5 V). The combined organic layers were washed with 9 wt % NaCl(aq) (225.0 L, 5 V) and concentrated to 2-4 V under reduced pressure. The MTBE was exchanged with THF (3×200.6 kg (5 V) under reduced pressure distillation to about 160 L (2-4 V). GC analysis of the solution indicated the criteria was achieved with MTBE s 1 area %.

THF (109.15 kg, 2.5 V) and water (13.00 kg, 0.3 V) were charged to the reactor, followed by Ba(OH)₂·8H₂O (13.95 kg, 0.52 eq. (0.515-0.525 eq.)). The reaction mixture was stirred for 30 hours at 42±5° C. The reaction was cooled to 25±5° C. and assayed by HPLC. The result of 2.4 area % Compound 17 did not meet the criteria of ≤2.0 area %. Ba(OH)₂·8H₂O (0.36 kg, 0.01 eq.) was charged to the reactor at 25±5° C. The temperature was adjusted, and the reaction mixture was stirred for three hours at 42±5° C. and then cooled to 25±5° C. HPLC analysis of the reaction met the criteria of ≤2.0 area % of Compound 17. The reaction mixture was concentrated to 140 L (2-4 V) and then azeotropically dried with THF (7×200.25 kg, 5 V) to a final volume of 100 L (2-4 V). The reaction met the KF endpoint of ≤0.5 wt %. n-heptane (460.60 kg, 15 V) was charged to a second reactor. The THF solution of Compound 2 was charged dropwise to the n-heptane. The resulting slurry was stirred for 30 minutes and then concentrated to 295 L (6-7 V). Additional n-heptane (91.50 kg, 3 V) was charged to the reactor. GC analysis of the reaction indicated the criteria was met of THF≤5 area %. The temperature was adjusted to 25±5° C. and the reaction mixture was stirred for five hours. The solids were isolated by centrifuge, washed once with n-heptane (62.05 kg, 2 V), and dried at 40±5° C. to afford 39.96 kg (85.91 moles) off-white solid.

¹H NMR (400 MHz, d6-DMSO, 25° C.)
δ4.36 (dd, J=10.4, 2.4 Hz, 1H), 4.08-4.22 (m, 2H), 3.40-3.51 (m, 1H), 3.01-3.07 (m, 2H), 2.76 (d, J=4.8 Hz, 3H), 2.21 (s, 6H), 1.84-2.21 (m, 4H), 1.36 (s, 6H), 0.94-0.98 (m, 3H), 0.71-0.77 (m, 3H)

### Part 2—Alternative Synthesis of Compound 2 (Barium Salt

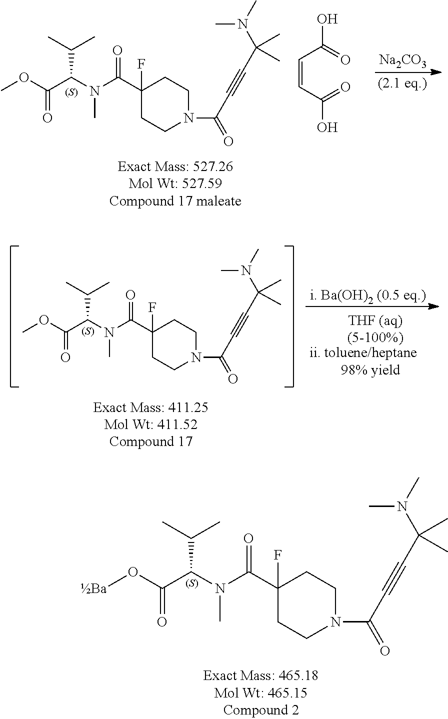

MTBE (7.0 V, 246.55 kg), water (10 V, 476.5 kg), Compound 17 maleate (1.0 eq., 47.56 kg) and Na₂CO₃ (2.1 eq., 20.15 kg) were charged to a reactor at 20±5° C. Additional MTBE (1.0 V, 35.25 kg) was charged as a rinse to the same reactor. The mixture was stirred under nitrogen until all the solids were dissolved. The phases were separated. The isolated lower lean aqueous layer was extracted with MTBE (5 V, 176.35 kg), and the layers were separated. The combined organic layers were washed with 9 wt % NaCl(aq) (5 V, 285.9 kg) and concentrated to 2-4 V under reduced pressure. The MTBE was exchanged with THF (3×−214 kg (5 V) under reduced pressure distillation to 2-4 V. THF (3.0 V, 112.20 kg) water (0.3 V, 14.25 kg), and Ba(OH)₂·8H₂O (0.52 eq., 13.80 kg) were charged to the reactor. The reaction mixture was stirred for 36 hours at 42±5° C., cooled to 25±5° C. and was analyzed by HPLC. The criteria of ≤1.0 area % Compound 17 was not achieved. Ba(OH)₂·8H₂O (0.05 eq., 1501.7 kg) was charged to the reactor at 25±5° C. The reaction mixture was stirred for 18 hours at 42±5° C., cooled to 25±5° C. and analyzed by HPLC. The reaction met the criteria of ≤1.0 area % Compound 17. 1 M HCl(aq) (0.08 eq., 7.21 kg) was charged dropwise to the reaction mixture at 20±5° C. The reaction mixture was further stirred for 30 min at 20±5° C. The THF was swapped with toluene (2×204 kg (5 V) and further concentrated to 2-4 V. Analysis of the mixture met the KF criteria of <0.5%. n-heptane (322.85 kg, 10 V) was charged dropwise at 20±5° C. and the mixture was concentrated to 6-7 V. Additional n-heptane (100.25 kg, 3 V) was charged to the reactor. The reaction mixture was stirred for an additional 5 hours at 20±5° C. The solids were isolated by filtration, washed twice with n-heptane (33 kg, 1 V), and dried at 45±10° C. for 28 hours to afford 40.76 kg of white solid in 93.0% yield and with 98.5 area % purity.

¹H NMR (400 MHz, d6-DMSO, 23° C.)
δ4.37 (d, J=9.2 Hz, 1H), 4.08-4.22 (m, 2H), 3.44-3.50 (m, 1H), 3.00-3.10 (m, 2H), 2.69-2.76 (m, 3H), 1.84-2.30 (m, 4H), 2.21 (s, 6H), 1.36 (d, J=4.0 Hz, 6H), 0.94-0.98 (m, 3H), 0.71-0.77 (m, 3H)

### Part 3—Synthesis of Compound 2 (Free Act

The barium salt of Compound 2 can be converted to the free acid form or to various amine salts.

The free acid and/or amine salts may offer the ability to enhance the purity and/or reactivity of Compound 2.

To prepare the free acid of Compound 2, 1.0 eq of Compound 2½ Ba and 4 V of water are charged into a reactor at 20±5° C. The reactor is then charged with H₂SO₄ aqueous (1 M, 0.53 eq). The resulting mixture is stirred for 2 h at 20±5° C. then filtered to remove BaSO₄ by celite. The filtrates are concentrated to 4-5 V. Solvent switching with toluene (5.0 V) is carried out eight times. The mixture is concentrated to dryness to afford a sticky oil. The oil and 10 V of MTBE are charged into a reactor and stirred for 6 h at 20±5° C. for filtration.

The free acid of Compound 2 is functional in the coupling with Compound 15 as described in Example 8, below. Other salts of Compound 2 can be formed using maleic acid, tartaric acid, and chiral amine (non-racemic) bases including cinchonidine, quinine, and (S)-cyclohexylethylamine. These may provide an advantage for isolation, purification, stability or coupling.

### Example 10. Synthetic Procedure for Compound A

### Part 1—Synthesis of Compound 12—(1²M)-tert-butyl (S)-2-((S)-2-(((benzyloxy)carbonyl)amino)-3-((S)-3-((3-(5-bromo-1-eth yl-2-(2-((S)-1-methoxyethyl) pyridin-3-yl)-1H-indol-3-yl)-2,2-dimethylpropoxy)carbonyl)tetrahydropyridazin-1(2H)-yl)-3-oxopropyl)morpholine-4-carboxylate

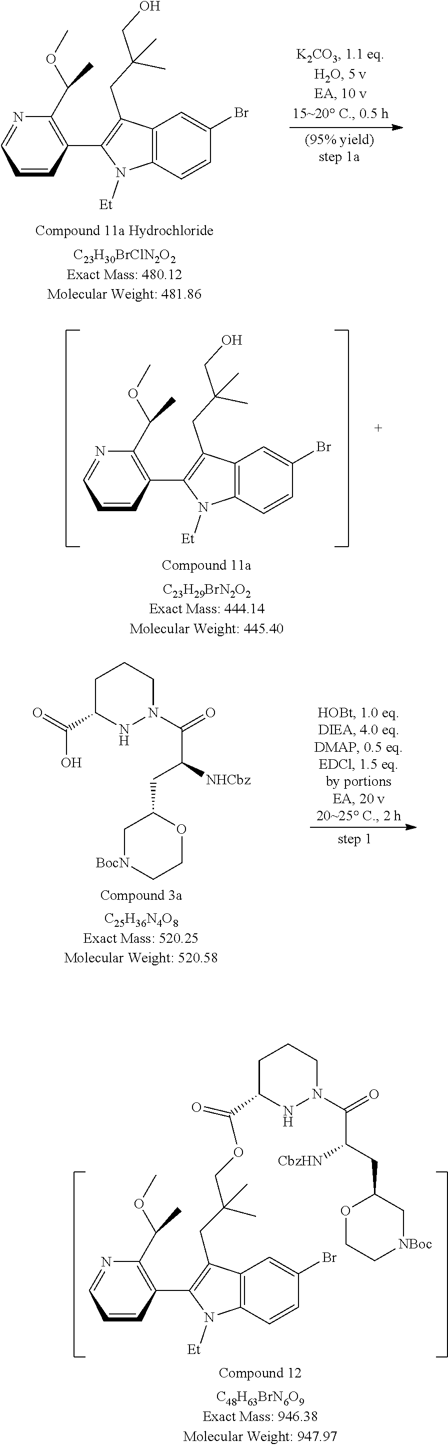

To a 500 L glass-lined reactor was charged Compound 11a Hydrochloride (8.60 kg, 1 eq) and EtOAc (79 kg, 10 v). The mixture was cooled to 10-20° C. under nitrogen and treated with an aqueous K₂CO₃ solution (46 kg, 6.5 wt %, 1.1 eq). The mixture was agitated at 10-20° C. for 0.5 h. The phases were separated, and the organic phase was washed twice with water (46 kg×2) and once with brine (46 kg, 25 wt %). The resulting organic layer was concentrated to afford Compound 11a as a EtOAc solution, which was used for step 1 directly.

To the concentrated solution of Compound 11a in EtOAc was charged Compound 3a (10.6 kg, 1.15 eq), HOBt (2.4 kg, 1.0 eq), DMAP (1.1 kg, 0.5 eq) and EtOAc (112 kg). The mixture was agitated at 15-25° C. under nitrogen and DIPEA (9.2 kg, 4.0 eq) was charged in portions over 3 h. EDCl (5.3 kg, 1.5 eq) was charged in portions over 8 h at 15-25° C. under agitation. Reaction monitoring by HPLC showed the reaction is complete.

The crude reaction mixture was washed successively with 10 wt % citric acid aqueous solution (70 kg×2), 5 wt % NaHCO₃ aqueous solution (70 kg×2) and brine (58 kg). The crude organic solution was concentrated under reduced pressure at NMT 40° C. to give Compound 12 as an EtOAc solution (102 kg, 19.0 wt %), which was used for the next step without further purification.

LRMS (ESI+)
Calculated for C₄₈H₆₄BrN₆O₉(M+H⁺): 947.38
Found: 946.1; 948.1

### Part 2—Synthesis of Compound 13—(1²M)-3-(5-bromo-1-ethyl-2-(2-((S)-1-methoxyethyl)pyridin-3-yl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate

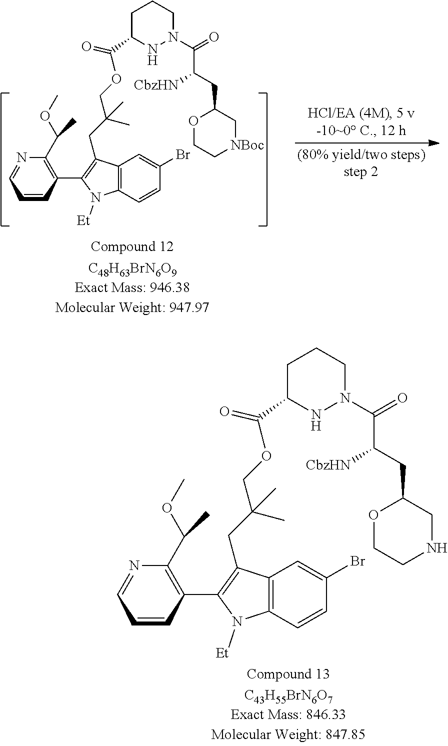

To a 500 L glass-lined reactor was charged Compound 12 EtOAc solution (102 kg, 19 wt %, 1.0 eq) and it was cooled to −10-0° C. under nitrogen. An HCl solution in EtOAc (100 kg, 4 M) was charged to the reactor at −10-0° C. under nitrogen over 2 h. The mixture was agitated at −10-0° C. under nitrogen for 10-16 h. Reaction monitoring showed the reaction was complete.

EtOAc (37 kg) was charged to reaction mixture and excess HCl was removed by sparging the mixture with nitrogen at −10-0° C. for 2-4 h. The slurry was filtered, and the wet cake was washed EtOAc (35 kg). The crude solid was slurried with EtOAc (167 kg) and cooled to −10-10° C. under nitrogen. NaHCO₃ aqueous solution (168 kg, 7 wt %) was charged slowly while maintaining the temperature at −10-−10° C. The mixture was warmed to 15-25° C. and agitated for 2 h. The phases were separated, and the organic layer was washed successively with NaHCO₃ aqueous solution (168 kg, 7 wt %) and brine (168 kg, 25 wt %). The crude organic solution was concentrated under reduced pressure at NMT 40° C. to give crude product solution in EtOAc. It was then purified by crystallization with a mixture of EtOAc and cyclohexane to give Compound 13 as a solid (12.35 kg, 85.2 wt %, 80% assay corrected yield over 2 steps)
Calculated for C₄₃H₅₆BrN₆O₇ (M+H⁺): 847.33
Found: 846.0, 848.0

### Part 3a—Synthesis of Compound 14—(1²M)-benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3) pyridazinacycloundecaphane-4-yl)carbamate

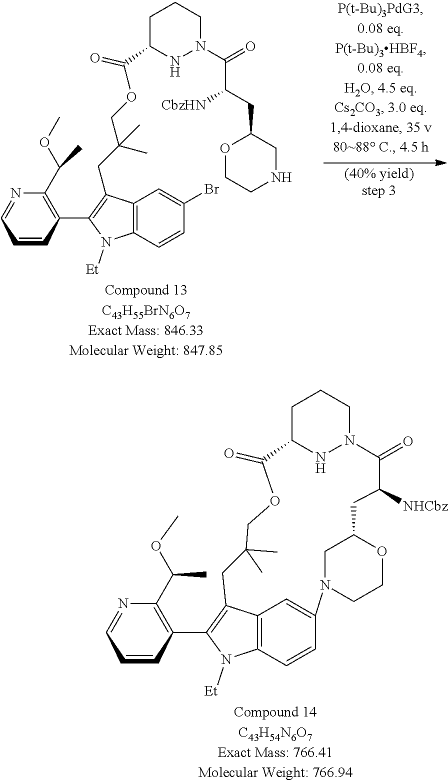

To a 1000 L reactor was charged Compound 13 (10 kg, 1.0 eq), CS₂CO₃ (11.5 kg, 3.0 eq), water (1.00 kg) and 1,4-dioxane (383 kg, 35 v). The mixture was agitated for 30 min and then it was degassed by bubbling nitrogen for 4 h under agitation. P(t-Bu)₃ HBF₄ (0.26 kg, 0.08 eq) and P(t-Bu)₃ Pd G3 (0.51 kg, 0.08 eq) were charged to the reactor and the reaction mixture was further degassed by bubbling nitrogen for 2 h. The reaction mixture was agitated at 80-88° C. for 4-7 h. Reaction monitoring by HPLC showed the reaction was complete.

The crude reaction mixture was cooled to 15-35° C. before EtOAc (42 kg) and water (48 kg) were charged. The mixture was agitated at 15-25° C. for 60 min. The phases were separated. The organic phase was washed with brine (53 kg, 25 wt %) and was concentrated under reduced pressure at NMT 50° C. to give a concentrated solution (65 kg). The concentrated solution was charged into water (55 kg) under agitation. The resulting slurry was agitated at 15-25° C. for 4 h and filtered. The solids were washed with water and recrystallized from a mixture of MeOH and water to afford Compound 14 as a solid (3.35 kg, 40% assay corrected yield)

¹H NMR (400 MHz, d6-DMSO, 25° C.)
δ8.75 (d, J=4 Hz, 1H), 7.81 (d, J=4 Hz, 1H), 7.42 (d, J=8 Hz, 1H), 7.29-7.35 (m, 5H), 7.18 (d, J=8 Hz, 1H), 6.99-7.05 (m, 2H), 7.50 (dd, J=8, 4 Hz, 1H). 5.30 (bd, J=4 Hz, 2H), 4.99 (q, J=12 Hz, 2H), 4.10-4.30 (m, 3H), 3.82-3.95 (m, 2H), 3.58-3.76 (m, 5H), 3.43 (d, J=12 Hz, 1H), 3.22 (d, J=12 Hz, 1H), 3.05 (s, 3H), 2.65-2.85 (m, 4H), 1.99 (d, J=4 Hz, 2H), 1.81 (d, J-Hz, 2H), 1.50-1.70 (m, 2H), 1.40 (d, J=4 Hz, 3H), 0.97 (bs, 3H), 0.68 (bs, 3H), 0.57 (bs, 3H)

### Part 3b—Alternative Method to Synthesize Compound 14

The following alternative synthesis of Compound 14 provides for the use of lower cost, more readily available substrates and a more robust process.

A 1000 mL three-necked round-bottomed flask was charged with milled C₅₂CO₃ (23.06 g, 3.0 equiv.), anisole (300 mL, 15 V), bis(tri-t-butylphosphine)palladium (O) (1.2 g, 2.36 mmol, 0.1 equiv.) and water (1.3 mL, 3.0 equiv.) under an inert atmosphere. The reaction mixture was sparged subsurface with an inert gas for 10-15 min and was heated to 85° C. (84-87° C.). Compound 13 (20 g) as a solution in anisole (10 V) was charged over 4-5 hours under an inert atmosphere. After one additional hour, reaction monitoring by HPLC showed the reaction was complete. The reaction mixture was cooled to 50° C. and filtered through Solka-Floc® cellulose Filter aid to remove insoluble solids. The reactor and finer aid were washed with anisole (2 V).

A portion of the filtrate (9.33 V=⅓ of the total volume), water (112 mL, 5.6 V) and seeds (1 wt %) were charged to a reactor to achieve a 62.5:37.5 ratio of anisole to water. The slurry was aged with moderate to high agitation for no less than 12 hours at room temperature. To the resulting slurry was charged simultaneously the filtrate (4.6 V=⅙ of the total original volume) and water (2.8 V) over one hour at the same rate. The mixture was concentrated under reduced pressure at 50° C. to 15 V. The partial simultaneous charge of filtrate (4.6 V=⅙ of the total original volume) and water (2.8 V) followed by concentration was repeated three additional times. The resulting concentrated slurry was aged at 50° C. for no less than 12 hours and further concentrated under reduced pressure at 50° C. to 10 V. If necessary, additional water was charged to achieve a ratio of 8:2 anisole/water. The slurry was concentrated further under reduced pressure at 50° C. to 7 V. If necessary, additional water was charged to achieve a ratio of 9:1 anisole/water. The slurry was aged at 50° C. for three hours, cooled to 10° C. over six hours, and aged at 10° C. for no less than 12 hours. The solids were isolated by filtration, washed seven times with heptane (1 V) and dried under reduced pressure at 40° C.

### Part 4—Synthesis of Compound 15—(1²M)-(2²S,6³S,4S)-4-amino-1¹-ethyl-1²-(2-((S)-1-methoxyethyl)pyridin-3-yl-10,10-dimethyl-6¹,6²,6³,6⁴,6⁵,6⁶-hexahydro-1¹H-8-oxa-2(4,2)-morpholina-1(5,3)-indola-6(1,3) pyridazinacycloundecaphane-5,7-dione

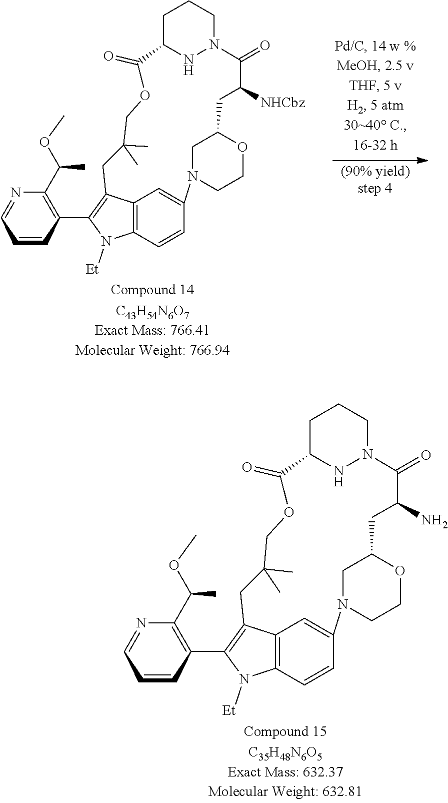

To a 50 L pressure reactor was charged Compound 14 (2.26 kg, 1.0 eq), 10% Pd/C (0.32 kg), THE (10.0 kg, 5 v) and MeOH (4.5 kg, 2.5 v). It was agitated at 5 atm H₂, 30-40° C. The atmosphere was exchanged by venting and refill with fresh H₂ several times during the course of the reaction.

After the reaction was complete as monitored by HPLC, the atmosphere was exchanged with N₂, and the reaction mixture was filtered through a pad of diatomite. The spent filter aid was washed with a mixture of THF and MeOH. The combined filtrates were then concentrated under reduced pressure at NMT 40° C. and diluted with EtOAc (186 kg). The mixture was further concentrated under reduced pressure at NMT 30° C. to ˜67-146 L. The addition of EtOAc and concentration was repeated 2 more times to remove the THF and MeOH. The resulting crude production solution in EtOAc was agitated with SiliaMetS Thiol) (0.732 kg) at 15-25° C. for 15 h. After filtration, the crude product in EtOAc was concentrated under reduced pressure at NMT 30° C., followed with crystallization from a mixture of EtOAc and n-heptane to afford Compound 15 as a solid.

HRMS (ESI+)
Calculated for C₃₅H₄₉N₈O₅ (M+H): 633.37
Found: 633.1

1H NMR (400 MHz, d6-DMSO, 24° C.)
δ8.74 (d, J=4 Hz, 1H), 7.78 (d, J=8 Hz, 1H), 7.50 (dd, J=4, 4 Hz, 1H), 7.43 (d, J=8 Hz, 1H), 7.04 (d, J=8 Hz, 1H), 7.00 (s, 1H), 5.14 (bd, J=12 Hz, 1H), 4.51 (bt, J=4 Hz, 1H), 4.36 (bd, J=12 Hz, 1H), 4.25-4.16 (m, 2H), 4.06-3.93 (m, 3H), 3.74-3.56 (m, 5H), 3.45 (d, J=12 Hz, 1H), 3.25 (d, J=12 Hz, 1H), 3.14 (s, 3H), 2.78-2.65 (m, 4H), 2.03 (bd, J=12 Hz, 1H), 1.99 (s, 1H), 1.84-1.51 (m, 5H), 1.39 (d, J=4 Hz, 3H), 1.18 (t, J=8 Hz, 1H), 0.90-0.84 (m, 3H), 0.72 (bs, 3H), 0.43 (bs, 3H)

### Part 5—Synthesis of Compound A—(1²M)-1-(4-(dimethylamino)-4-methylpent-2 ynoyl)-N-((2S)-1-(((2²S,6³S,4S)-1¹-ethyl-12-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3) pyridazinacycloundecaphane-4-yl)amino)-3-methyl-1-oxobutan-2 yl)-4-fluoro-N-methylpiperidine-4-carboxamide

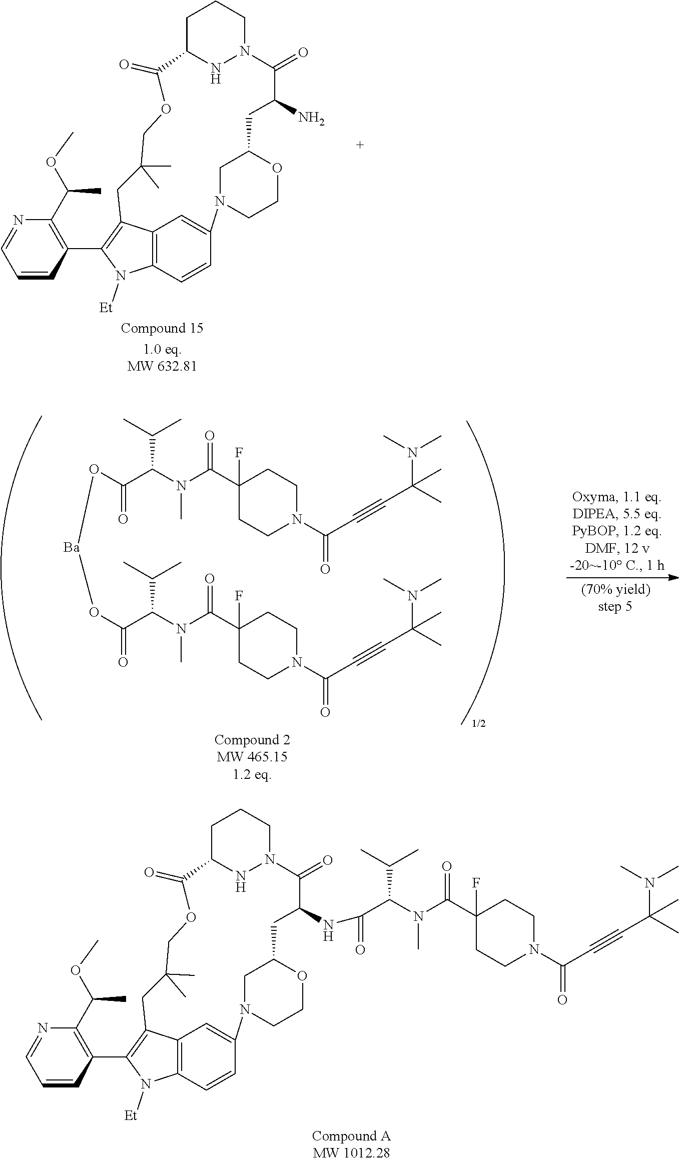

To a 50 L glass reactor was charged Compound 15 (1.91 kg, 1.0 eq) and DMF (13.9 kg). The mixture was agitated at 20-30° C. until all of the solids were dissolved. Compound 2 (1.70 kg, 1.2 eq) and DMF (3.8 kg) were charged. The mixture was agitated at 20-30° C. until all of the solids were dissolved. DIPEA (2.20 kg, 5.50 eq) was charged at 20-30° C. and the mixture was cooled to −20-−10° C. under agitation. Ethyl cyanoglyoxylate-2-oxime (Oxyma) (0.48 kg, 1.1 eq) was charged to the reactor and the reaction mixture was agitated at −20 to −10° C. for 30 min. PyBOP was charged as a DMF solution (1.89 kg dissolved in 3.62 kg DMF, 1.2 eq) to the reactor at −20 to −10° C. in </=1 h. The reaction mixture was agitated at −20 to −10° C. for 1-3 h. Reaction monitoring by HPLC showed the reaction was complete.

The crude reaction mixture was diluted with EtOAc (3.6 kg) and partitioned with a mixture of EtOAc (65 kg) and brine (25 wt %, 132 kg). The biphasic mixture was agitated at 20-30° C. for 1 h and filtered through a pad of diatomite. EtOAc (11 kg) was used to rinse the reactor and spent filter aid wet cake. The combined filtrates were allowed to stand for 1 h before the phases were separated. The organic layer was washed once with brine (25 wt %, 90 kg×2). HCl (0.6 M aqueous solution, 71 kg) was charged to the isolated organic layer at 5-20° C. The biphasic mixture was agitated at 10-20° C. for 1 h. The phases were separated, and the upper lean organic phase was extracted with HCl (0.6 M aqueous solution, 30 kg). The combined rich aqueous phases were washed three times with EtOAc (34 kg×3). To the washed aqueous phase was charged EtOAc (34 kg) and the pH was adjusted to pH 9-10 by charging Na₂CO₃ aqueous solution (30 wt %) at 10-20° C. The mixture was agitated at 10-20° C. for 1 h and the phases were separated. The lean aqueous phase was extracted with EtOAc (34 kg), and the combined rich organic phases were washed twice with brine (25 wt %, 90 kg×2). The resulting organic layer was then washed with a solution of acetic acid in brine (prepared by dissolving 0.23 kg glacial acetic acid and 8 kg 25 wt % brine in 115 kg water) (39 kg×2), a solution of Na₂CO₃ in brine (prepared by dissolving 1.3 kg Na₂CO₃ and 8 kg 25 wt % brine in 31 kg water) (40 kg), and brine (25 wt %, 92 kg), respectively. The crude organic solution was then treated with CUNO® by filtering through a cartridge and the filtrate was concentrated under reduced pressure at NMT 40° C. to ˜30 L. The crude residue was then crystallized by charging n-heptane (57 kg) with seed (0.040 kg).

The crude product was then further purified by recrystallization with a mixture of EtOAc and n-Heptane to give purified Compound A as a white solid.

HRMS (ESI+)
Calculated for C₅₅H₇₈FN₉O₈(M+H): 1012.6036
Found: 1012.6065

1H NMR (400 MHz, CD₃OD, 23° C.)
δ8.71 (dd J=4.8, 1.6 Hz, 1H), 7.83 (d, J=7.2 Hz, 1H), 7.50 (dd, J=8.0, 4.8 Hz, 1H), 7.39 (d, J=8.0 Hz, 1H), 7.11 (s, 1H), 7.07 (dd, J=9.0, 2.0 Hz, 1H), 5.67 (d, J=8.8 Hz, 1H), 4.62 and 4.52 (d, J=10.0 Hz, 1H, as rotamers), 4.46 (d, J=12.4 Hz, 1H), 4.28-4.37 (m, 2H), 4.24 (q (J=6.4 Hz, 1H), 4.12-4.17 (m, 1H), 3.50-4.00 (m, 9H), 3.28 (d, J=10.8 Hz, 1H), 3.10-3.20 and 2.90-3.00 (m, 8H, as rotamers), 2.60-2.80 (m, 4H), 2.35 (s, 3H), 2.34 (s, 3H), 2.05-2.35 (m, 7H), 1.85-1.95 (m, 2H), 1.60-1.73 (m, 2H), 1.46 (s, 3H), 1.45 (s, 3H), 1.44 (s, 3H), 0.96 and 1.02 (d, J=6.4 Hz, 3H, as rotamers), 1.00-1.10 (m, 3H), 0.83 and 0.87 (dd, J=6.8, 2.0 Hz, 3H, as rotamers), (0.77 (bs, 3H), 0.64 (bs, 3H)

### Part 6—Direct Synthesis of Compound a without Isolation of Compound 15

Alternatively, Compound A can be prepared in a telescoping process that avoids isolation and characterization of Compound 15. This process allows for a more robust and cost-effective process.

To a pressure reactor was charged Compound 14 (34.8 kg), 10% Pd/C, water-wet (0.20×), THE (5 V), and iPrOH (3 V). The mixture was agitated under hydrogen (0.5 MPa) at 30-40° C. for 32 hours. After the reaction was complete as monitored by HPLC, the atmosphere was exchanged with N₂, and the reaction mixture was filtered through a pad of diatomaceous earth to remove the spent Pd/C catalyst. The spent filter aid was washed with a mixture of THF and iPrOH. The combine filtrates and a silica-thiol scavenger (0.1×) were mixed together at 20-30° C. for 14 hours to reduce residual palladium. The mixture was filtered and the spent scavenger was washed with a mixture of THF and iPrOH. The filtrate was concentrated under reduced pressure to 3 V and diluted with DMF (3 V).

To the concentrated solution of Compound 15 was charged Compound 2 (0.60 equiv.), DIPEA (5.5 equiv.) and DMF (9 V). The mixture was cooled to between −50 and −10° C. In quick rapid succession, Oxyma (1.1 equiv.) and PyBOP (1.2 equiv.) as a solution in DMF (2 V) were added, in no more than 30 minutes at −40 to −30° C. The reaction mixture was agitated at −20 to −10° C. for 3 h. Reaction monitoring by HPLC showed the reaction was complete.

The crude reaction mixture was added dropwise over one hour to a mixture of EtOAc (20 kg) and 25 wt % brine (30 V). The biphasic mixture was agitated at 20-30° C. for 1 h and was filtered through a diatomaceous earth (0.5×). The spent filter aid was washed with EtOAc (1 V). The combined organic phases were washed with twice with 25 wt % brine (20 V) and, at 10-20° C., was partitioned with 0.6M HCl(aq) (18 V). The lean organic layer was extracted with 0.6M HCl(aq) (8 V). The combined rich aqueous phases were washed three times with EtOAc (10 V), partitioned with EtOAc (10 V) and neutralized to pH 9.0-10.0 with 30 wt % Na₂CO₃(aq) (˜7.2×) at 10-20° C. The lean aqueous layer was washed with EtOAc (10 V). The combined organics layers were washed successively twice with 25 wt % brine (20 V), twice with 0.2 wt % AcOH, 3 wt % NaCl in water (10 V), once 3 wt % Na₂CO₃, 5 wt % NaCl in water (10 V), once with 25 wt % brine (20 V) and filtered through a carbon filter. The reactor and filter were rinsed with EtOAc. The combined filtrates were concentrated under reduced pressure at <40° C. to 7-8 V and charged to a mixture of n-heptane (21.6 V) and seeds (0.7 wt %) at 25-35° C. The resulting slurry was cooled to between and 25° C. over four hours, aged for eight hours, and filtered. The solids were washed with a mixture of 3:1 n-heptane and EtOAc (2.0×) and dried under reduced pressure at 40-50 for 30 hours to afford crude Compound A as a white solid.

### Part 7—Purification of Compound A

Crude Compound A was dissolved in EtOAc (4.0 V), and treated with water (0.08×) and DIPEA (0.015×). The solution was stirred at 35 to 45° C. for 30 minutes. n-Heptane (4.0 V) was charged over 30 minutes at 35 to 45° C. Seeds (1 wt %) were charged at 35 to 45° C. The mixture was stirred at 35 to 45° C. for eight hours. The resulting slurry was cooled to between 2° and 30° C. over six hours, aged with agitation for 30 minutes, warmed to 35 to 45° C. over one hour, and aged for 30 minutes. The temperature cycle was repeated one or two more times. A mixture of 10.5:1 v/v n-heptane/EtOAc (6.1×) was charged over six hours. The slurry was cooled to between 5 and 15° C. over eight hours, aged for eight hours, and filtered. The solids were washed twice with a mixture of 2.5:1 v/v n-heptane/EtOAc (2.0 V) and dried under reduced pressure at 50° C. to afford Compound A as a white solid.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

1. A barium salt of Compound 1:

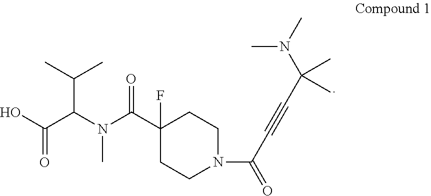 [SMILES: CC(C)C(C(=O)O)N(C)C(=O)C1(F)CCN(C(=O)C#CC(C)(C)N(C)C)CC1]

2. The barium salt of claim 1, wherein the barium salt has a 2:1 ratio of carboxylic acid:barium.

3. The barium salt of claim 1 or 2, wherein the barium salt has the structure of Compound 2:

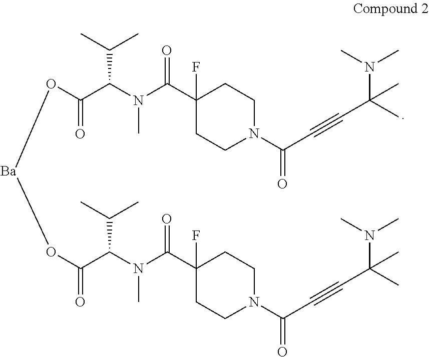

4. A compound having the structure of Compound 3: 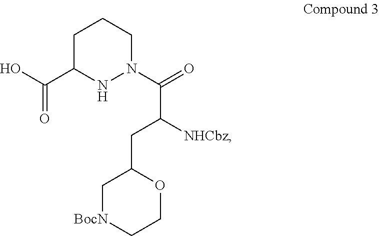 [SMILES: CC(C)(C)OC(=O)N1CCOC(CC(NC(=O)OCc2ccccc2)C(=O)N2CCCC(C(=O)O)N2)C1]
or a salt thereof.

5. The compound of claim 4, wherein the compound has the structure of Compound 3a: 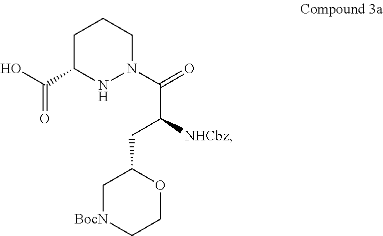 [SMILES: CC(C)(C)OC(=O)N1CCO[C@@H](C[C@H](NC(=O)OCc2ccccc2)C(=O)N2CCC[C@@H](C(=O)O)N2)C1]
or a salt thereof.

6. A method of preparing Compound 4a, the method comprising contacting Compound 4 with one or more ketoreductase enzymes to produce Compound 4a:

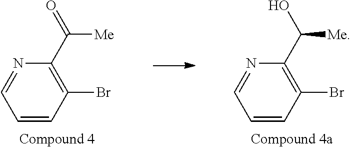 [SMILES: CC(=O)c1ncccc1Br ; C[C@H](O)c1ncccc1Br]

7. The method of claim 6, wherein Compound 4a is formed in at least 85% yield.

8. A tetramethylethylenediamine (TMEDA) salt of Compound 5:

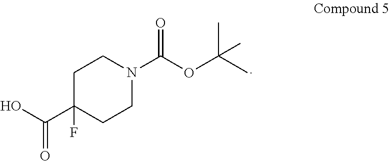 [SMILES: CC(C)(C)OC(=O)N1CCC(F)(C(=O)O)CC1]

9. A 1,4-diazabicyclo[2.2.2]octane (DABCO) salt of Compound 5:

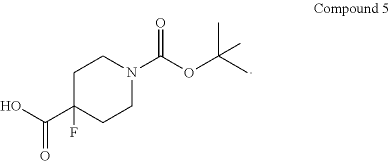 [SMILES: CC(C)(C)OC(=O)N1CCC(F)(C(=O)O)CC1]

10. A method of preparing Compound 6: 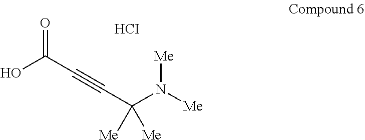 [SMILES: CN(C)C(C)(C)C#CC(=O)O ; "HCI"]
the method comprising:
a) bis-N-methylating Compound 7 to form Compound 7c: 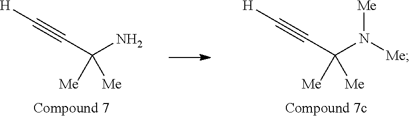 [SMILES: C#CC(C)(C)N ; C#CC(C)(C)N(C)C]
b) carboxylating Compound 7c to form Compound 7a: 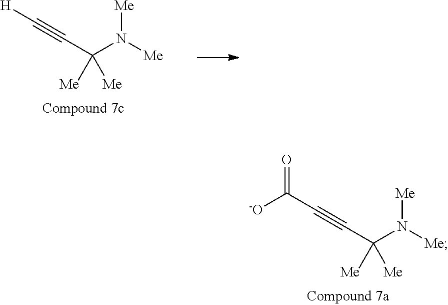
c) protonating Compound 7a to form Compound 7b: 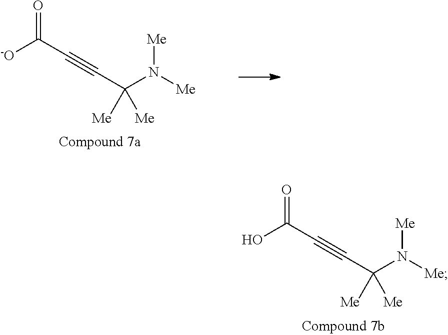
and
d) protonating compound 7b to form Compound 6:

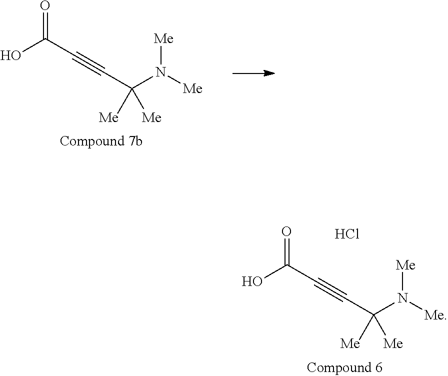 [SMILES: CN(C)C(C)(C)C#CC(=O)O ; CN(C)C(C)(C)C#CC(=O)O ; Cl]

11. The method of claim 10, wherein the bis-N-methylating step (a) comprises contacting Compound 7 with an alkylating agent and a reducing agent.

12. A method of preparing Compound 8: 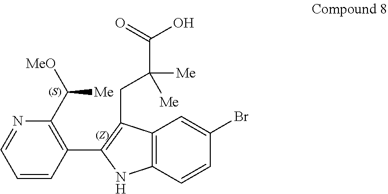
the method comprising contacting Compound 9 and Compound 10: 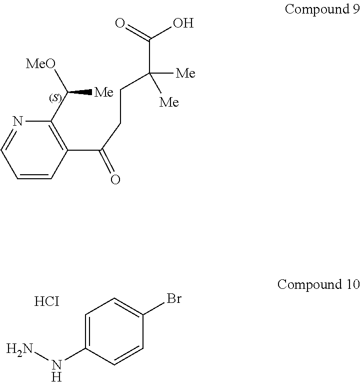
in the presence of an acid and water.

13. A method of preparing Compound 8: 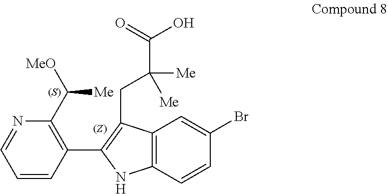
the method comprising:
a) coupling Compound 4b and Compound 20 to form Compound 21: 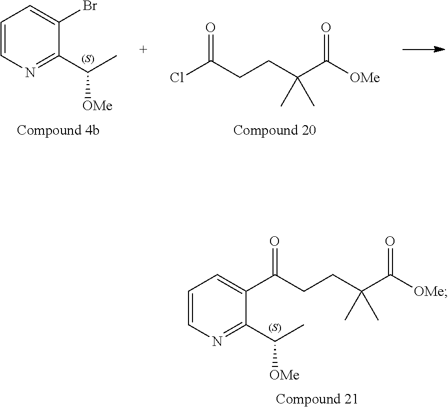
and
b) contacting Compound 21 and Compound 10 to form Compound 8:

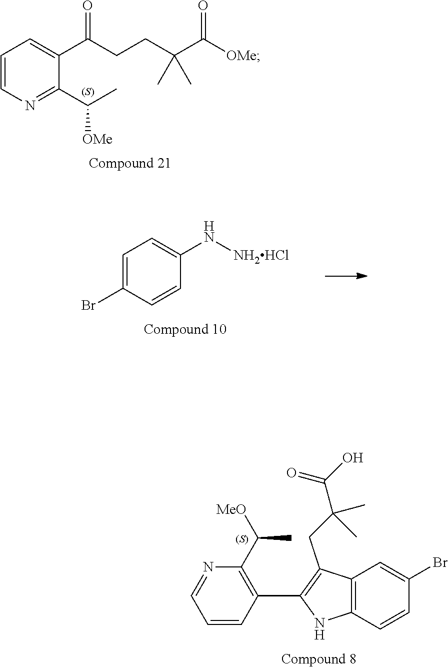

14. A hemisulfate (2:1 ratio of Compound 8:sulfate) salt of compound 8:

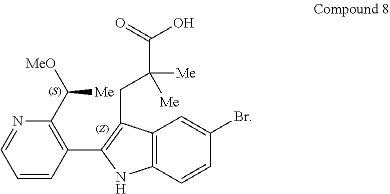

15. A method of preparing the hemisulfate salt of claim 14, the method comprising contacting a free base of Compound 8 with sulfuric acid.

16. A method of preparing Compound 11a and 11b, 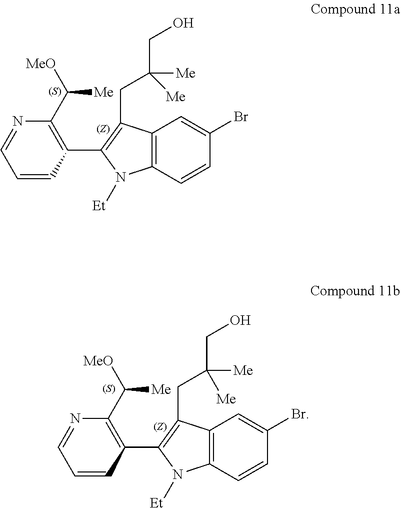
the method comprising:
a) reducing Compound 22 to form Compound 23 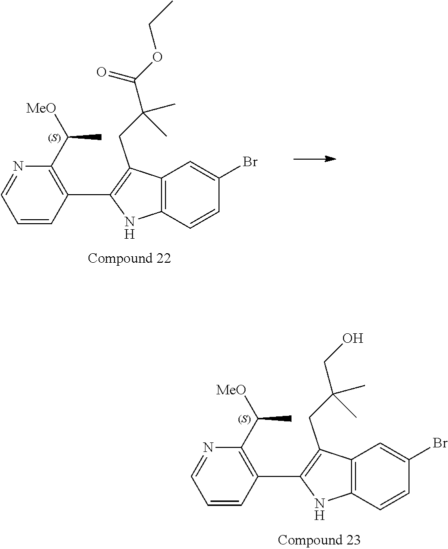
b) alkylating Compound 23 to form a mixture of Compound 11a and 11b

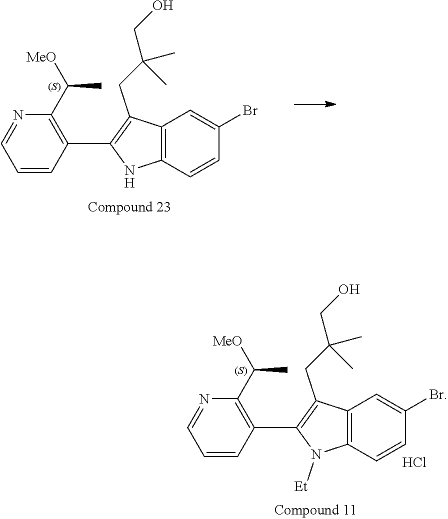

17. A method of preparing Compound 11a and 11b, 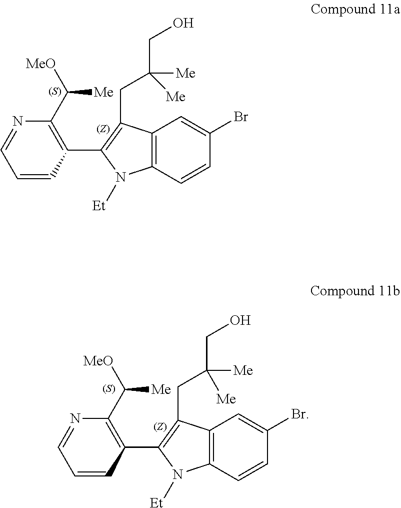
the method comprising:
a) alkylating Compound 22 to form Compound 24 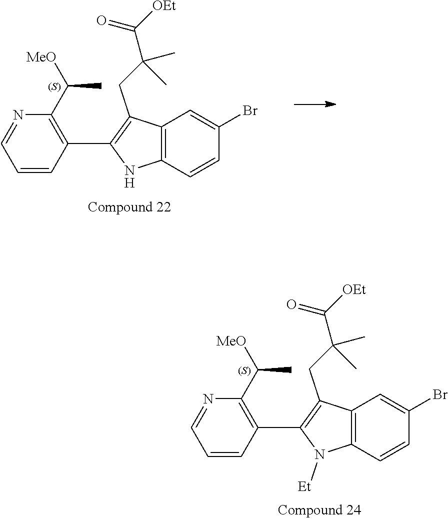
and
b) reducing Compound 24 to form a mixture of Compound 11a and 11b

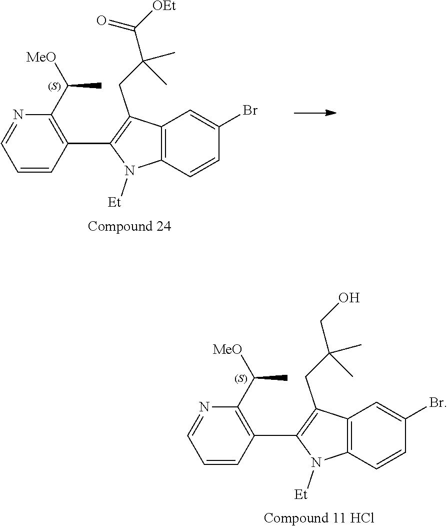

18. A method of separating Compound 11a and Compound 11b: 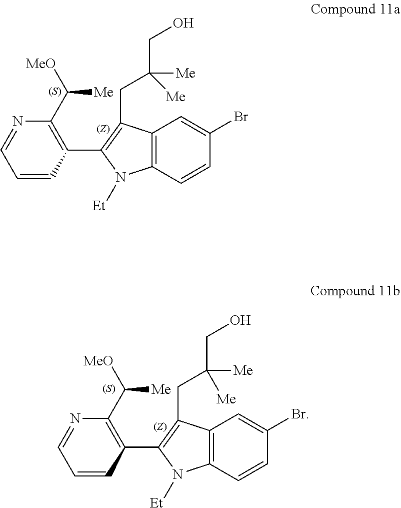
the method comprising:
a) heating a mixture of Compound 11a and Compound 11b in a solvent or mixture of solvents for a period of time;
b) forming a mixture of a salt of Compound 11a and a salt of Compound 11b; and
c) separating the mixture of the salt of Compound 11a and Compound 11b.

19. The method of claim 18, wherein the separating is carried out as a flow process.

20. A method of preparing Compound 3a: 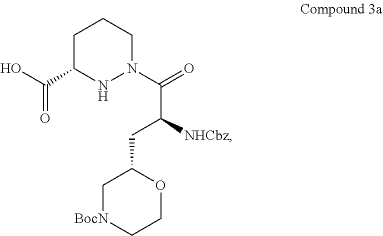 [SMILES: CC(C)(C)OC(=O)N1CCO[C@@H](C[C@H](NC(=O)OCc2ccccc2)C(=O)N2CCC[C@@H](C(=O)O)N2)C1]
the method comprising:
a) deprotonating compound 3b with a chiral base, thereby forming a diastereomeric salt of compound 3b: 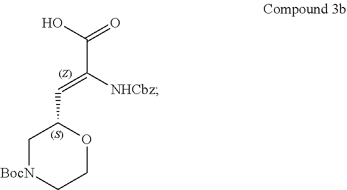
reducing the diastereomeric salt of Compound 3b to form Compound 3c: 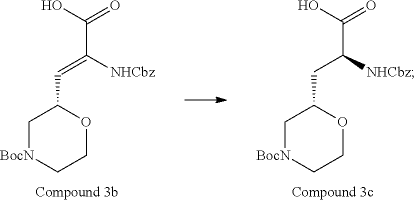 [SMILES: CC(C)(C)OC(=O)N1CCO[C@@H](/C=C(\NC(=O)OCc2ccccc2)C(=O)O)C1 ; CC(C)(C)OC(=O)N1CCO[C@@H](C[C@H](NC(=O)OCc2ccccc2)C(=O)O)C1]
c) coupling Compound 3c with Compound 3d, or a salt thereof, to form Compound 3e; 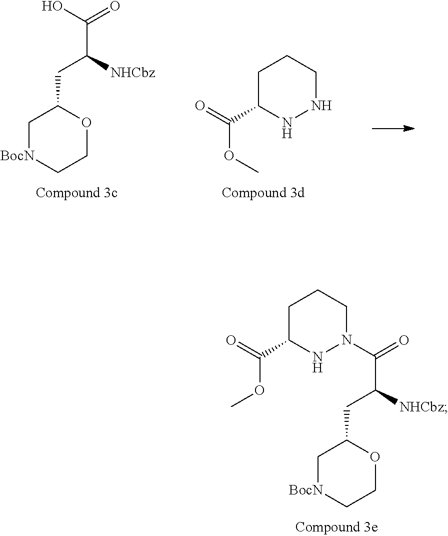
and
d) hydrolyzing Compound 3e to form Compound 3a:

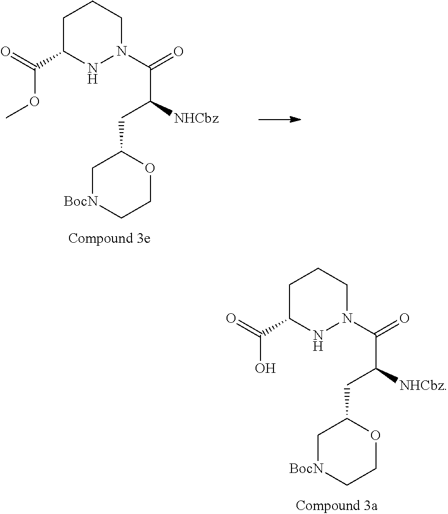

21. A method of preparing Compound A: 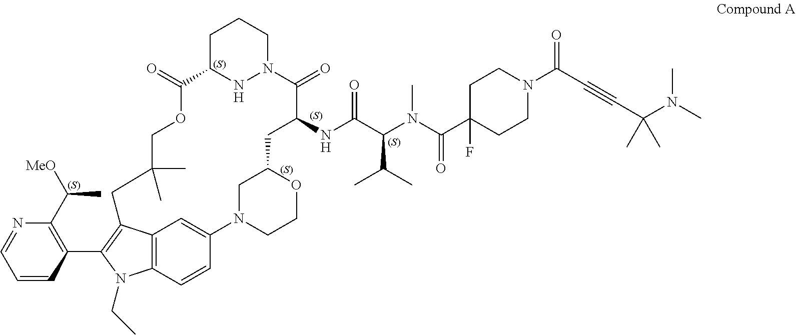
the method comprising:
a) coupling Compound 11a and Compound 3a to form Compound 12: 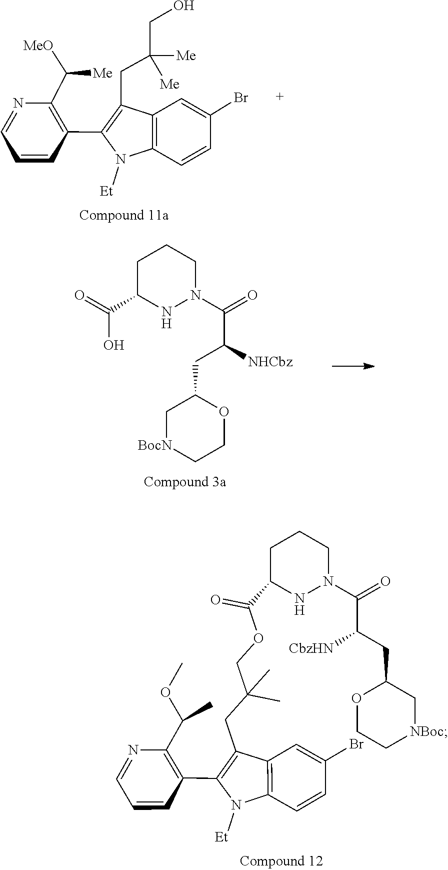
b) deprotecting Compound 12 to form Compound 13: 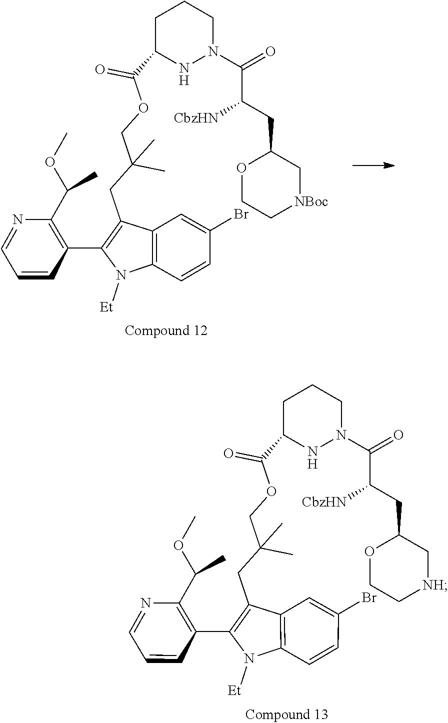
c) coupling Compound 13 to form Compound 14: 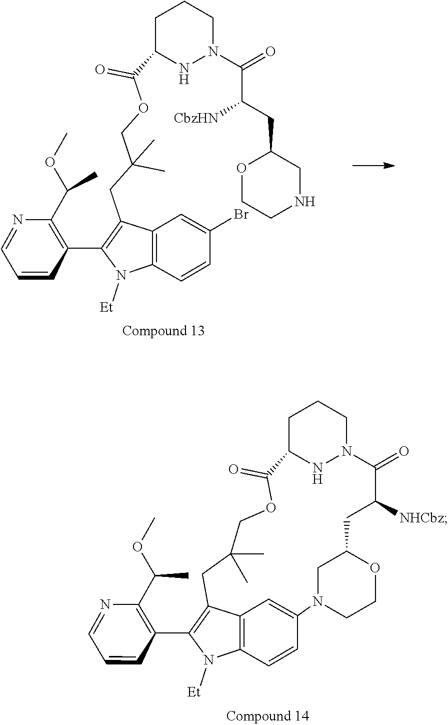
f) deprotecting of Compound 14 to form Compound 15: 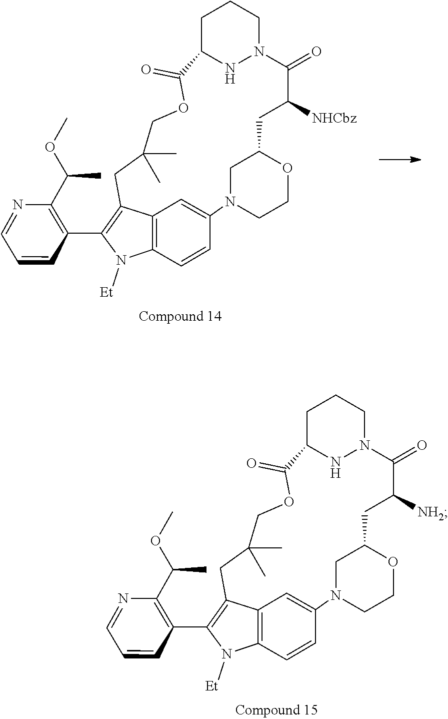
and
e) coupling Compound 15 and Compound 2 to form Compound A:

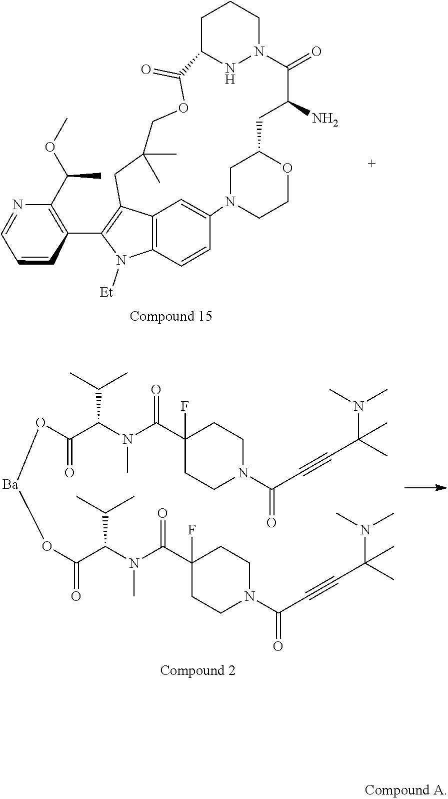

22. A compound having the structure of Compound 12: 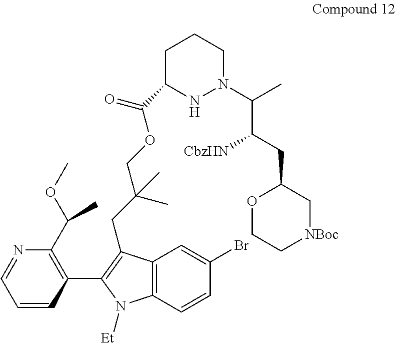
or a salt thereof.

23. A compound having the structure of Compound 13: 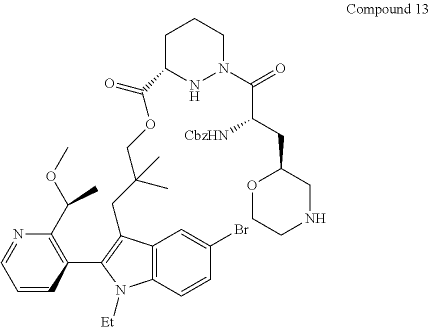
or a salt thereof.
